SEQUENCE LISTING (100 sequences)

SEQ ID NO: 1 - protein, 16 aa, Mus sp.
RSSQSLVHRNGNTYLH

SEQ ID NO: 2 - protein, 7 aa, Mus sp.
TVSNRFS

SEQ ID NO: 3 - protein, 9 aa, Mus sp.
SQSSHVPPT

SEQ ID NO: 4 - protein, 5 aa, Mus sp.
NYGVN

SEQ ID NO: 5 - protein, 17 aa, Mus sp.
WINPNTGEPTFDDDFKG

SEQ ID NO: 6 - protein, 11 aa, Mus sp.
SRGKNEAWFAY

SEQ ID NO: 7 - protein, 5 aa, Mus sp.
NYGMN

SEQ ID NO: 8 - protein, 17 aa, Mus sp.
WINTYTREPTYADDFKG

SEQ ID NO: 9 - protein, 12 aa, Mus sp.
DITAVVPTGFDY

SEQ ID NO: 10 - protein, 11 aa, Mus sp.
RASENIYSNLA

SEQ ID NO: 11 - protein, 7 aa, Mus sp.
AASNLAD

SEQ ID NO: 12 - protein, 9 aa, Mus sp.
QHFWTTPWA

SEQ ID NO: 13 - DNA, 21 bp, Artificial Sequence
  Description of Artificial Sequence Synthetic oligonucleotide
aatgcggcggtggtgacagta

SEQ ID NO: 14 - DNA, 21 bp, Artificial Sequence
  Description of Artificial Sequence Synthetic oligonucleotide
aagctcagcacacagaaagac

SEQ ID NO: 15 - DNA, 21 bp, Artificial Sequence
  Description of Artificial Sequence Synthetic oligonucleotide
uaaaaucuuccugcccacctt

SEQ ID NO: 16 - DNA, 21 bp, Artificial Sequence
  Description of Artificial Sequence Synthetic oligonucleotide
ggaagcuguuggcugaaaatt

SEQ ID NO: 17 - DNA, 21 bp, Artificial Sequence
  Description of Artificial Sequence Synthetic oligonucleotide
aagaccagccucuuugcccag

SEQ ID NO: 18 - DNA, 19 bp, Artificial Sequence
  Description of Artificial Sequence Synthetic oligonucleotide
ggaccaggcagaaaacgag

SEQ ID NO: 19 - DNA, 17 bp, Artificial Sequence
  Description of Artificial Sequence Synthetic oligonucleotide
cuaucaggaugacgcgg

SEQ ID NO: 20 - DNA, 21 bp, Artificial Sequence
  Description of Artificial Sequence Synthetic oligonucleotide
ugacacaggcaggcuugacuu

SEQ ID NO: 21 - DNA, 19 bp, Artificial Sequence
  Description of Artificial Sequence Synthetic oligonucleotide
ggtgaagaagggcgtccaa

SEQ ID NO: 22 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 23 - DNA, 20 bp, Artificial Sequence
  Description of Artificial Sequence Synthetic oligonucleotide
aggtggtgttaacagcagag

SEQ ID NO: 24 - DNA, 21 bp, Artificial Sequence
  Description of Artificial Sequence Synthetic oligonucleotide
aaggtggagcaagcggtggag

SEQ ID NO: 25 - DNA, 21 bp, Artificial Sequence
  Description of Artificial Sequence Synthetic oligonucleotide
aaggagttgaaggccgacaaa

SEQ ID NO: 26 - DNA, 21 bp, Artificial Sequence
  Description of Artificial Sequence Synthetic oligonucleotide
uauggagcugcagaggaugtt

SEQ ID NO: 27 - DNA, 49 bp, Artificial Sequence
  Description of Artificial Sequence Synthetic oligonucleotide
tttgaatatctgtgctgagaacacagttctcagcacagatattcttttt

SEQ ID NO: 28 - DNA, 29 bp, Artificial Sequence
  Description of Artificial Sequence Synthetic oligonucleotide
aatgagaaaagcaaaaggtgccctgtctc

SEQ ID NO: 29 - DNA, 21 bp, Artificial Sequence
  Description of Artificial Sequence Synthetic oligonucleotide
aaucaucaucaagaaagggca

SEQ ID NO: 30 - DNA, 21 bp, Artificial Sequence
  Description of Artificial Sequence Synthetic oligonucleotide
augacugucaggauguugctt

SEQ ID NO: 31 - DNA, 21 bp, Artificial Sequence
  Description of Artificial Sequence Synthetic oligonucleotide
gaacgaauccugaagacaucu

SEQ ID NO: 32 - DNA, 29 bp, Artificial Sequence
  Description of Artificial Sequence Synthetic oligonucleotide
aagcctggctacagcaatatgcctgtctc

SEQ ID NO: 33 - DNA, 21 bp, Artificial Sequence
  Description of Artificial Sequence Synthetic oligonucleotide
ugaccaucaccgaguuuautt

SEQ ID NO: 34 - DNA, 21 bp, Artificial Sequence
  Description of Artificial Sequence Synthetic oligonucleotide
aagtcggacgcaacagagaaa

SEQ ID NO: 35 - DNA, 21 bp, Artificial Sequence
  Description of Artificial Sequence Synthetic oligonucleotide
cuaccuuucuacggacgugtt

SEQ ID NO: 36 - DNA, 21 bp, Artificial Sequence
  Description of Artificial Sequence Synthetic oligonucleotide
ctgcctaaggcggatttgaat

SEQ ID NO: 37 - DNA, 21 bp, Artificial Sequence
  Description of Artificial Sequence Synthetic oligonucleotide
ttauuccuucuucgggaaguc

SEQ ID NO: 38 - DNA, 21 bp, Artificial Sequence
  Description of Artificial Sequence Synthetic oligonucleotide
aaccttctggaacccgcccac

SEQ ID NO: 39 - DNA, 19 bp, Artificial Sequence
  Description of Artificial Sequence Synthetic oligonucleotide
gagcatcttcgagcaagaa

SEQ ID NO: 40 - DNA, 19 bp, Artificial Sequence
  Description of Artificial Sequence Synthetic oligonucleotide
catgtggcaccgtttgcct

SEQ ID NO: 41 - DNA, 21 bp, Artificial Sequence
  Description of Artificial Sequence Synthetic oligonucleotide
aactaccagaaaggtatacct

SEQ ID NO: 42 - DNA, 21 bp, Artificial Sequence
  Description of Artificial Sequence Synthetic oligonucleotide
ucacaguguccuuuauguatt

SEQ ID NO: 43 - DNA, 21 bp, Artificial Sequence
  Description of Artificial Sequence Synthetic oligonucleotide
gcaugaaccggaggcccautt

SEQ ID NO: 44 - DNA, 19 bp, Artificial Sequence
  Description of Artificial Sequence Synthetic oligonucleotide
ccggacagttccatgtata

SEQ ID NO: 45 - protein, 44 aa, Homo sapiens
SHIQIPPGLTELLQGYTVEVLRQQPPDLVEFAVEYFTRLREARA

SEQ ID NO: 46 - protein, 45 aa, Homo sapiens
CGHIQIPPGLTELLQGYTVEVLRQQPPDLVEFAVEYFTRLREARA

SEQ ID NO: 47 - protein, 17 aa, Artificial Sequence
  Description of Artificial Sequence Synthetic peptide
QIEYLAKQIVDNAIQQA

SEQ ID NO: 48 - protein, 21 aa, Artificial Sequence
  Description of Artificial Sequence Synthetic peptide
CGQIEYLAKQIVDNAIQQAGC

SEQ ID NO: 49 - protein, 50 aa, Homo sapiens
SLRECELYVQKHNIQALLKDSIVQLCTARPERPMAFLREYFERLEKEEAK

SEQ ID NO: 50 - protein, 55 aa, Homo sapiens
MSCGGSLRECELYVQKHNIQALLKDSIVQLCTARPERPMAFLREYFERLEKEEAK

SEQ ID NO: 51 - protein, 23 aa, Homo sapiens
CGFEELAWKIAKMIWSDVFQQGC

SEQ ID NO: 52 - protein, 55 aa, Artificial Sequence
  Description of Artificial Sequence Synthetic polypeptide
GSGGGGSGGGGSHIQIPPGLTELLQGYTVEVLRQQPPDLVEFAVEYFTRLREARA

SEQ ID NO: 53 - protein, 29 aa, Artificial Sequence
  Description of Artificial Sequence Synthetic peptide
GSGGGGSGGGGSQIEYLAKQIVDNAIQQA

SEQ ID NO: 54 - protein, 51 aa, Homo sapiens
SLRECELYVQKHNIQALLKDVSIVQLCTARPERPMAFLREYFEKLEKEEAK

SEQ ID NO: 55 - protein, 54 aa, Homo sapiens
SLKGCELYVQLHGIQQVLKDCIVHLCISKPERPMKFLREHFEKLEKEENRQILA

SEQ ID NO: 56 - protein, 44 aa, Homo sapiens
SHIQIPPGLTELLQGYTVEVGQQPPDLVDFAVEYFTRLREARRQ

SEQ ID NO: 57 - protein, 44 aa, Homo sapiens
SIEIPAGLTELLQGFTVEVLRHQPADLLEFALQHFTRLQQENER

SEQ ID NO: 58 - protein, 44 aa, Artificial Sequence
  Description of Artificial Sequence Synthetic consensus polypeptide
XXIXIPPXLXXLLXXYXVXVLXXXPPXLVXFXVXYFXXLXXXXX

SEQ ID NO: 59 - protein, 17 aa, Artificial Sequence
  Description of Artificial Sequence Synthetic consensus peptide
XXXXXAXXIVXXAIXXX

SEQ ID NO: 60 - protein, 17 aa, Artificial Sequence
  Description of Artificial Sequence Synthetic peptide
QIEYVAKQIVDYAIHQA

SEQ ID NO: 61 - protein, 17 aa, Artificial Sequence
  Description of Artificial Sequence Synthetic peptide
QIEYKAKQIVDHAIHQA

SEQ ID NO: 62 - protein, 17 aa, Artificial Sequence
  Description of Artificial Sequence Synthetic peptide
QIEYHAKQIVDHAIHQA

SEQ ID NO: 63 - protein, 17 aa, Artificial Sequence
  Description of Artificial Sequence Synthetic peptide
QIEYVAKQIVDHAIHQA

SEQ ID NO: 64 - protein, 18 aa, Artificial Sequence
  Description of Artificial Sequence Synthetic peptide
PLEYQAGLLVQNAIQQAI

SEQ ID NO: 65 - protein, 18 aa, Artificial Sequence
  Description of Artificial Sequence Synthetic peptide
LLIETASSLVKNAIQLSI

SEQ ID NO: 66 - protein, 18 aa, Artificial Sequence
  Description of Artificial Sequence Synthetic peptide
LIEEAASRIVDAVIEQVK

SEQ ID NO: 67 - protein, 18 aa, Artificial Sequence
  Description of Artificial Sequence Synthetic peptide
ALYQFADRFSELVISEAL

SEQ ID NO: 68 - protein, 17 aa, Artificial Sequence
  Description of Artificial Sequence Synthetic peptide
LEQVANQLADQIIKEAT

SEQ ID NO: 69 - protein, 17 aa, Artificial Sequence
  Description of Artificial Sequence Synthetic peptide
FEELAWKIAKMIWSDVF

SEQ ID NO: 70 - protein, 18 aa, Artificial Sequence
  Description of Artificial Sequence Synthetic peptide
ELVRLSKRLVENAVLKAV

SEQ ID NO: 71 - protein, 18 aa, Artificial Sequence
  Description of Artificial Sequence Synthetic peptide
TAEEVSARIVQVVTAEAV

SEQ ID NO: 72 - protein, 18 aa, Artificial Sequence
  Description of Artificial Sequence Synthetic peptide
QIKQAAFQLISQVILEAT

SEQ ID NO: 73 - protein, 16 aa, Artificial Sequence
  Description of Artificial Sequence Synthetic peptide
LAWKIAKMIVSDVMQQ

SEQ ID NO: 74 - protein, 24 aa, Artificial Sequence
  Description of Artificial Sequence Synthetic peptide
DLIEEAASRIVDAVIEQVKAAGAY

SEQ ID NO: 75 - protein, 18 aa, Artificial Sequence
  Description of Artificial Sequence Synthetic peptide
LEQYANQLADQIIKEATE

SEQ ID NO: 76 - protein, 20 aa, Artificial Sequence
  Description of Artificial Sequence Synthetic peptide
FEELAWKIAKMIWSDVFQQC

SEQ ID NO: 77 - protein, 17 aa, Artificial Sequence
  Description of Artificial Sequence Synthetic peptide
QIEYLAKQIPDNAIQQA

SEQ ID NO: 78 - protein, 25 aa, Artificial Sequence
  Description of Artificial Sequence Synthetic peptide
KGADLIEEAASRIVDAVIEQVKAAG

SEQ ID NO: 79 - protein, 25 aa, Artificial Sequence
  Description of Artificial Sequence Synthetic peptide
KGADLIEEAASRIPDAPIEQVKAAG

SEQ ID NO: 80 - protein, 25 aa, Artificial Sequence
  Description of Artificial Sequence Synthetic peptide
PEDAELVRLSKRLVENAVLKAVQQY

SEQ ID NO: 81 - protein, 25 aa, Artificial Sequence
  Description of Artificial Sequence Synthetic peptide
PEDAELVRTSKRLVENAVLKAVQQY

SEQ ID NO: 82 - protein, 25 aa, Artificial Sequence
  Description of Artificial Sequence Synthetic peptide
PEDAELVRLSKRDVENAVLKAVQQY

SEQ ID NO: 83 - protein, 25 aa, Artificial Sequence
  Description of Artificial Sequence Synthetic peptide
PEDAELVRLSKRLPENAVLKAVQQY

SEQ ID NO: 84 - protein, 25 aa, Artificial Sequence
  Description of Artificial Sequence Synthetic peptide
PEDAELVRLSKRLPENAPLKAVQQY

SEQ ID NO: 85 - protein, 25 aa, Artificial Sequence
  Description of Artificial Sequence Synthetic peptide
PEDAELVRLSKRLVENAVEKAVQQY

SEQ ID NO: 86 - protein, 25 aa, Artificial Sequence
  Description of Artificial Sequence Synthetic peptide
EEGLDRNEEIKRAAFQIISQVISEA

SEQ ID NO: 87 - protein, 25 aa, Artificial Sequence
  Description of Artificial Sequence Synthetic peptide
LVDDPLEYQAGLLVQNAIQQAIAEQ

SEQ ID NO: 88 - protein, 25 aa, Artificial Sequence
  Description of Artificial Sequence Synthetic peptide
QYETLLIETASSLVKNAIQLSIEQL

SEQ ID NO: 89 - protein, 25 aa, Artificial Sequence
  Description of Artificial Sequence Synthetic peptide
LEKQYQEQLEEEVAKVIVSMSIAFA

SEQ ID NO: 90 - protein, 25 aa, Artificial Sequence
  Description of Artificial Sequence Synthetic peptide
NTDEAQEELAWKIAKMIVSDIMQQA

SEQ ID NO: 91 - protein, 25 aa, Artificial Sequence
  Description of Artificial Sequence Synthetic peptide
VNLDKKAVLAEKIVAEAIEKAEREL

SEQ ID NO: 92 - protein, 25 aa, Artificial Sequence
  Description of Artificial Sequence Synthetic peptide
NGILELETKSSKLVQNIIQTAVDQF

SEQ ID NO: 93 - protein, 25 aa, Artificial Sequence
  Description of Artificial Sequence Synthetic peptide
TQDKNYEDELTQVALALVEDVINYA

SEQ ID NO: 94 - protein, 25 aa, Artificial Sequence
  Description of Artificial Sequence Synthetic peptide
ETSAKDNINIEEAARFLVEKILVNH

SEQ ID NO: 95 - protein, 44 aa, Artificial Sequence
  Description of Artificial Sequence Synthetic consensus polypeptide
XHIXIPPGLXELLQGYTXEVLRXQPPDLVEFAXXYFXXLXEXRX

SEQ ID NO: 96 - protein, 21 aa, Artificial Sequence
  Description of Artificial Sequence Synthetic peptide
CGQIEYLAKQIVDNAIQQAGC

SEQ ID NO: 97 - protein, 22 aa, Homo sapiens
RSQSRSRYYRQRQRSRRRRRRS

SEQ ID NO: 98 - protein, 4 aa, Artificial Sequence
  Description of Artificial Sequence Synthetic peptide
PKSC

SEQ ID NO: 99 - protein, 164 aa, Artificial Sequence
  Description of Artificial Sequence Synthetic polypeptide
QDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSCGGGGSLECGHIQIPPGLTELLQGYTVEVLRQQPPDLVEFAVEYFTRLREARAVEHHHHHH

SEQ ID NO: 100 - protein, 9 aa, Artificial Sequence
  Description of Artificial Sequence Synthetic peptide
NLVPMVATV

(19) United States
(12) Patent Application Publication
(10) Pub. No.: US 2013/0164214 A1
(43) Pub. Date: Jun. 27, 2013

(54) Antibody-Based Depletion of Antigen-Presenting Cells and Dendritic Cells

(71) Applicants: Chien-Hsing Chang, Downingtown, PA (US); David M. Goldenberg, Mendham, NJ (US)

(72) Inventors: Chien-Hsing Chang, Downingtown, PA (US); David M. Goldenberg, Mendham, NJ (US)

(73) Assignee: IMMUNOMEDICS, INC., Morris Plains, NJ (US)

(21) Appl. No.: 13/656,159

(22) Filed: Oct. 19, 2012

Related U.S. Application Data
(63) Continuation of application No. 13/074,351, filed on Mar. 29, 2011.
Application No. 13/074,351 is a continuation-in-part of application No. 13/567,226, filed on Aug. 6, 2012.
Application No. 13/567,226 is a division of application No. 13/004,349, filed on Jan. 11, 2011, now abandoned.
(60) Provisional application No. 61/319,902, filed on Apr. 1, 2010.
(60) Provisional application No. 61/329,282, filed on Apr. 29, 2010.
(60) Provisional application No. 61/293,846, filed on Jan. 11, 2010.
(60) Provisional application No. 61/323,001, filed on Apr. 12, 2010.
(60) Provisional application No. 61/374,449, filed on Aug. 17, 2010.

(51) Int. Cl. A61K 39/395; A61K 51/10; A61K 31/69; A61K 31/713; A61K 47/48

(52) U.S. Cl. CPC A61K 39/3955; A61K 31/713; A61K 47/48561; A61K 31/69; A61K 51/1096
USPC 424/1.49; 424/173.1; 424/133.1; 424/1.11; 424/178.1; 424/136.1; 424/183.1; 424/85.1; 424/85.7; 424/85.6; 424/85.5; 424/85.2; 424/135.1; 530/391.7; 530/387.3

(57) ABSTRACT

Disclosed herein are methods and compositions comprising anti-CD74 and/or anti-HLA-DR antibodies for treatment of GVHD and other immune dysfunction diseases. In preferred embodiments, the anti-CD74 and/or anti-HLA-DR antibodies are effective to deplete antigen-presenting cells, such as dendritic cells. Most preferably, administration of the therapeutic compositions depletes all subsets of APCs, including mDCs, pDCs, B cells and monocytes, without significant depletion of T cells. In alternative embodiments, administration of the therapeutic compositions suppresses proliferation of allo-reactive T cells, while preserving cytomegalovirus (CMV)-specific, CD8⁺ memory T cells. The compositions and methods provide a novel conditioning regimen for preventing aGVHD and/or treating chronic GVHD, without altering preexisting anti-viral immunity.

### CROSS-REFERENCE TO RELATED APPLICATIONS

This application is a continuation of U.S. patent application Ser. No. 13/074,351, filed Mar. 29, 2011, which claims the benefit under 35 U.S.C. 119(e) of U.S. Provisional Patent Appl. Nos. 61/319,902, filed Apr. 1, 2010, and 61/329,282, filed Apr. 29, 2010, the entire text of each of which is incorporated herein by reference. This application is a continuation-in-part of U.S. patent application Ser. No. 13/567,226, filed Aug. 6, 2012, which is a divisional of U.S. patent application Ser. No. 13/004,349, filed Jan. 11, 2011, which claims the benefit under 35 U.S.C. 119(e) of U.S. Provisional Patent Appl. Nos. 61/293,846, filed Jan. 11, 2010, 61/323,001, filed Apr. 12, 2010, and 61/374,449, filed Aug. 17, 2010.

### SEQUENCE LISTING

The instant application contains a Sequence Listing which has been submitted in ASCII format via EFS-Web and is hereby incorporated by reference in its entirety. Said ASCII copy, created on Mar. 23, 2011, is named IMM328US.txt and is 37,022 bytes in size.

### FIELD OF THE INVENTION

The present invention concerns compositions and methods of use of antibodies, antibody fragments, immunoconjugates and/or other targeting molecules for treatment of immune dysfunction diseases, including but not limited to graft-versus-host disease (GVHD) and organ transplant rejection. Preferably, the compositions and methods relate to use of anti-CD74 and/or anti-HLA-DR antibodies, immunoconjugates or fragments thereof to deplete antigen-presenting cells (APCs), such as dendritic cells (DCs). More preferably, administration of the therapeutic compositions results in significant depletion of myeloid DCs type 1 (mDC1) and type 2 (mDC2) and mild depletion of B cells, without significant depletion of plasmacytoid DCs (pDCs), monocytes or T cells. Most preferably, administration of the therapeutic compositions depletes all subsets of APCs, including mDCs, pDCs, B cells and monocytes, without significant depletion of T cells. In alternative embodiments, administration of the therapeutic compositions suppresses proliferation of allo-reactive T cells, while preserving cytomegalovirus (CMV)-specific, CD8⁺ memory T cells. The compositions and methods provide a novel conditioning regimen for maximally preventing acute graft-versus-host disease (aGVHD) without altering preexisting anti-viral immunity.

### BACKGROUND

Allogeneic hematopoietic stem cell transplantation (allo-HSCT) is a curative therapy for many hematological malignancies, but is frequently followed by aGVHD, the leading cause of mortality and morbidity in allo-HSCT patients (Socie & Blazar, Blood 114, 4327-4336, 2009). The major initiator of aGVHD is host antigen-presenting cells (APCs) that are residual after preparative conditioning (Shlomchik et al. Science 285:412-415, 1999; Chakraverty & Sykes, Blood 110:9-17, 2007). Current conditioning regimens incorporating anti-CD52 monoclonal antibody (alemtuzumab) effectively reduce aGVHD (Kottaridis et al. Blood 96:2419-2425, 2000), but result in cytomegalovirus (CMV) reactivation and impaired immune reconstitution (Pérez-Simón et al. Blood 100:3121-3127, 2002; Chakrabarti et al. Blood 99:4357-4363, 2002).

Despite the use of non-myeloablative or reduced-intensity conditioning regimens, GVHD remains a major and life-threatening complication for allo-HSCT (Landfried, et al. Curr Opin Oncol 21:S39-S41, 2009). It is well documented that among residual host APCs the critical subset for initiating aGVHD is dendritic cells (DCs) (Duffner et al. J Immunol 172:7393-7398, 2004; Durakovic et al. J Immunol 177:4414-4425, 2006). Either host myeloid DCs (mDCs) or plasmacytoid DCs (pDCs) alone are sufficient to induce GVHD (Koyama et al. Blood 113:2088-2095, 2009). Donor APCs, especially mDCs, also contribute to the development of GVHD (Matte et al. Nat Med 10:987-992, 2004; Markey et al. Blood 113:5644-5649, 2009). Depletion of DCs has been an effective approach to reduce or abrogate GVHD (Merad et al. Nat Med 10:510-517, 2004; Zhang et al. J Immunol 169:7111-8, 2002; Wilson et al. J Exp Med 206:387-398, 2009).

In contrast to T-cell depletion, which is well-established in controlling GVHD (Poyton, Bone Marrow Transplant 3:265-279, 1988; Champlin, Hematol Oncol Clin North Am 4:687-98, 1990), but is associated with increased viral infection and tumor relapse (Chakraverty et al. Bone Marrow Transplant 28:827-34, 2001; Wagner et al. Lancet 366:733-741, 2005), depletion of DCs to prevent GVHD does not have these complications (Wilson et al. J Exp Med 206:387-398, 2009). The humanized anti-CD52 antibody, alemtuzumab (Campath-1H), and its homologous rat anti-human CD52 antibody, Campath-1G, deplete both DCs and T cells (Klangsinsirikul et al. Blood 99:2586-2591, 2002; Hale et al. Blood 92:4581-90, 1998; Buggins et al. Blood 100:1715-1720, 2002; Morris et al. Blood 102:404-406, 2003), and effectively prevent GVHD after allo-HSCT (Willemze et al. Bone Marrow Transplant 9:255-61, 1992; Durakovic et al. J Immunol 177:4414-4425, 2006). Alemtuzumab is routinely incorporated in conditioning regimens for GVHD prevention but at the cost of CMV reactivation and impaired immune reconstitution due to T-cell depletion (Pérez-Simón et al. Blood 100:3121-3127, 2002; Chakrabarti et al. Blood 99:4357-4363, 2002).

Besides DCs, B cells and monocytes are two other major subsets of circulating APCs. Accumulating evidence has demonstrated that B cells are involved in the pathogenesis of acute and chronic GVHD (Shimabukuro-Vornhagen et al. Blood 114:4919-4927, 2009), and that B-cell depleting therapy is effective in prevention and treatment of GVHD (Alousi et al. Leuk Lymphoma 51:376-389, 2010). The anti-CD20 antibody, rituximab, when included in the conditioning regimen, reduces the incidence of aGVHD (Christopeit et al. Blood 113:3130-3131, 2009). Monocytes may also be involved in the pathogenesis of GVHD, since higher numbers of blood monocytes before conditioning are associated with greater risk of aGVHD (Arpinati et al. Biol Blood Marrow Transplant 13:228-234, 2007). In addition, the proteosome inhibitor, bortezomib, which efficiently depletes monocytes (Arpinati et al. Bone Marrow Transplant 43:253-259, 2009), is active in controlling acute and chronic GVHD (Sun et al. Proc Natl Acad Sci USA 101:8120-8125, 2004).

Because each subset of APCs is involved in the pathogenesis of aGVHD, a need exists in the field for methods and compositions to deplete all APC subsets during the preparative conditioning for allo-HSCT. This need remains unfulfilled by current treatment regimens.

### SUMMARY

The present invention concerns improved compositions and methods of use of antibodies against APCs in general and DCs in particular for the treatment of aGVHD. A variety of antigens associated with dendritic cells are known in the art, including but not limited to CD209 (DC-SIGN), CD34, CD74, CD205, TLR 2 (toll-like receptor 2), TLR 4, TLR 7, TLR 9, BDCA-2, BDCA-3, BDCA-4, and HLA-DR. Although in preferred embodiments the antibodies or fragments thereof of use are targeted to CD74 or HLA-DR, the skilled artisan will realize that antibodies against other DC-associated antigens can be used within the scope of the claimed method, either alone or in combination with other anti-CD antibodies. Antibodies against CD74 and HLA-DR include the anti-CD74 hLL1 antibody (milatuzumab) and the anti-HLA-DR antibody hL243 (also known as IMMU-114) (Berkova et al., 2010, Expert Opin. Investig. Drugs 19:141-49; Burton et al., 2004, Clin Cancer Res 10:6605-11; Chang et al., 2005, Blood 106:4308-14; Griffiths et al., 2003, Clin Cancer Res 9:6567-71; Stein et al., 2007, Clin Cancer Res 13:5556s-63s; Stein et al., 2010, Blood 115:5180-90).

Many examples of anti-CD74 antibodies are known in the art and any such known antibody or fragment thereof may be utilized. In a preferred embodiment, the anti-CD74 antibody is an hLL1 antibody (also known as milatuzumab) that comprises the light chain complementarity-determining region (CDR) sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). A humanized LL1 (hLL1) anti-CD74 antibody suitable for use is disclosed in U.S. Pat. No. 7,312,318, incorporated herein by reference from Col. 35, line 1 through Col. 42, line 27 and FIG. 1 through FIG. 4. However, in alternative embodiments, other known and/or commercially available anti-CD74 antibodies may be utilized, such as LS-B1963, LS-B2594, LS-B1859, LS-B2598, LS-05525, LS-C44929, etc. (LSBio, Seattle, Wash.); LN2 (BIOLEGEND®, San Diego, Calif.); PIN.1, SPM523, LN3, CerCLIP.1 (ABCAM®, Cambridge, Mass.); At14/19, Bu45 (SEROTEC®, Raleigh, N.C.); 1D1 (ABNOVA®, Taipei City, Taiwan); 5-329 (EBIOSCIENCE®, San Diego, Calif.); and any other antagonistic anti-CD74 antibody known in the art.

The anti-CD74 antibody may be selected such that it competes with or blocks binding to CD74 of an LL1 antibody comprising the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6). Alternatively, the anti-CD74 antibody may bind to the same epitope of CD74 as an LL1 antibody.

Many examples of anti-HLA-DR antibodies are also known in the art and any such known antibody or fragment thereof may be utilized. In a preferred embodiment, the anti- HLA-DR antibody is an hL243 antibody (also known as IMMU-114) that comprises the heavy chain CDR sequences CDR1 (NYGMN, SEQ ID NO:7), CDR2 (WINTYIREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). A humanized L243 anti-HLA-DR antibody suitable for use is disclosed in U.S. Pat. No. 7,612,180, incorporated herein by reference from Col. 46, line 45 through Col. 60, line 50 and FIG. 1 through FIG. 6. However, in alternative embodiments, other known and/or commercially available anti- HLA-DR antibodies may be utilized, such as 1D10 (apolizumab) (Kostelny et al., 2001, Int J Cancer 93:556-65); MS-GPC-1, MS-GPC-6, MS-GPC-8, MS-GPC-10, etc. (U.S. Patent No. 7,521,047); Lym-1, TAL 8.1, 520B, ML11C11, SPM289, MEM-267, TAL 15.1, TAL 1B5, G-7, 4D12, Bra30 (Santa Cruz Biotechnology, Inc., Santa Cruz, Calif.); TAL 16.1, TU36, C120 (ABCAM®, Cambridge, Mass.); and any other anti- HLA-DR antibody known in the art.

The anti-HLA-DR antibody may be selected such that it competes with or blocks binding to HLA-DR of an L243 antibody comprising the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12). Alternatively, the anti- HLA-DR antibody may bind to the same epitope of HLA-DR as an L243 antibody.

The anti-CD74 and/or anti-HLA-DR antibodies or fragments thereof may be used as naked antibodies, alone or in combination with one or more therapeutic agents. Alternatively, the antibodies or fragments may be utilized as immunoconjugates, attached to one or more therapeutic agents. (For methods of making immunoconjugates, see, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, the Examples section of each incorporated herein by reference.) Therapeutic agents may be selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide molecule (e.g., an antisense molecule or a gene) or a second antibody or fragment thereof.

The therapeutic agent may be selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

The therapeutic agent may comprise a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{112m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

The therapeutic agent may be an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

An immunomodulator of use may be selected from the group consisting of a cytokine, a stem cell growth factor, a lymphotoxin, a hematopoietic factor, a colony stimulating factor (CSF), an interferon (IFN), erythropoietin, thrombopoietin and combinations thereof. Exemplary immunomodulators may include IL-1, IL-2, IL-3, IL-6, IL-10, IL-12, IL-18, IL-21, interferon-α, interferon-β, interferon-γ, G-CSF, GM-CSF, and mixtures thereof.

Exemplary anti-angiogenic agents may include angiostatin, endostatin, basculostatin, canstatin, maspin, anti-VEGF binding molecules, anti-placental growth factor binding molecules, or anti-vascular growth factor binding molecules.

In certain embodiments, the antibody or fragment may comprise one or more chelating moieties, such as NOTA, DOTA, DTPA, TETA, Tscg-Cys, or Tsca-Cys. In certain embodiments, the chelating moiety may form a complex with a therapeutic or diagnostic cation, such as Group II, Group III, Group IV, Group V, transition, lanthanide or actinide metal cations, Tc, Re, Bi, Cu, As, Ag, Au, At, or Pb.

In some embodiments, the antibody or fragment thereof may be a human, chimeric, or humanized antibody or fragment thereof. A humanized antibody or fragment thereof may comprise the complementarity-determining regions (CDRs) of a murine antibody and the constant and framework (FR) region sequences of a human antibody, which may be substituted with at least one amino acid from corresponding FRs of a murine antibody. A chimeric antibody or fragment thereof may include the light and heavy chain variable regions of a murine antibody, attached to human antibody constant regions. The antibody or fragment thereof may include human constant regions of IgG1, IgG2a, IgG3, or IgG4.

In certain preferred embodiments, the anti-CD74 and/or anti-HLA-DR complex may be formed by a technique known as dock-and-lock (DNL) (see, e.g., U.S. Pat. Nos. 7,521,056; 7,527,787; 7,534,866; 7,550,143 and 7,666,400, the Examples section of each of which is incorporated herein by reference.) Generally, the DNL technique takes advantage of the specific and high-affinity binding interaction between a dimerization and docking domain (DDD) sequence derived from the regulatory subunit of human cAMP-dependent protein kinase (PKA) and an anchor domain (AD) sequence derived from any of a variety of AKAP proteins. The DDD and AD peptides may be attached to any protein, peptide or other molecule. Because the DDD sequences spontaneously dimerize and bind to the AD sequence, the DNL technique allows the formation of complexes between any selected molecules that may be attached to DDD or AD sequences. Although the standard DNL complex comprises a trimer with two DDD-linked molecules attached to one AD-linked molecule, variations in complex structure allow the formation of dimers, trimers, tetramers, pentamers, hexamers and other multimers. In some embodiments, the DNL complex may comprise two or more antibodies, antibody fragments or fusion proteins which bind to the same antigenic determinant or to two or more different antigens. The DNL complex may also comprise one or more other effectors, such as a cytokine or PEG moiety.

Also disclosed is a method for treating and/or diagnosing a disease or disorder that includes administering to a patient a therapeutic and/or diagnostic composition that includes any of the aforementioned antibodies or fragments thereof. Typically, the composition is administered to the patient intravenously, intramuscularly or subcutaneously at a dose of 20-5000 mg. In preferred embodiments, the disease or disorder is an immune dysregulation disease, an autoimmune disease, organ-graft rejection or graft-versus-host disease. More preferably, the disease is aGVHD.

Exemplary autoimmune diseases include acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis.

In particularly preferred embodiments, administration of the anti-CD74 and/or anti-HLA-DR antibodies or fragments thereof can deplete all subsets of APCs, but not T cells, from human peripheral blood mononuclear cells (PBMCs), including myeloid DCs (mDCs), plasmacytoid DCs (pDCs), B cells, and monocytes. Most preferably, the antibodies or fragments suppress the proliferation of allo-reactive T cells in mixed leukocyte cultures while preserving CMV-specific, CD8⁺ memory T cells.

### BRIEF DESCRIPTION OF THE DRAWINGS

The following Figures are provided to illustrate exemplary, but non-limiting, preferred embodiments of the invention.

FIG. 1. Milatuzumab, but not its Fab fragment fusion protein, selectively depletes myeloid DCs in human PBMCs. Human PBMCs were incubated with 5 μg/ml milatuzumab, control antibodies, or medium only, for 3 days. The effect of each treatment on APC subsets was evaluated by co-staining the cells with PE-labeled anti-CD14 and anti-CD19, in combination with APC-labeled anti-BDCA-1, for analysis of mDC1, or a mixture of FITC-labeled anti-BDCA-2 and APC-labeled anti-BDCA-3 for simultaneous analysis of mDC2 and pDCs, respectively. 7-AAD was added before flow cytometric analyses. PBMCs were gated to exclude the debris and dead cells on the basis of their forward and side scatter characteristics. The subpopulations of PBMCs were gated as follows: monocytes, CD14⁺SSC^{medium}; B cells, CD19⁺SSC^{low}; non-B lymphocytes (T and null cells), CD19⁻CD14⁻SSC^{low}; mDC1, CD14⁻CD19⁻BDCA-1⁺. The live cell fraction of each cell population was determined by measuring 7-AAD^{neg} cells. (FIG. 1A) Mean percentages of live mDC1, B cells, monocytes, and non-B lymphocytes in PBMCs following antibody treatments, n=6 donors. (FIG. 1B) Mean percentages of live mDC2 and pDCs in PBMCs following antibody treatments, n=7 donors. Error bars, SD; **, P<0.05; and ***P<0.01 vs. hMN-14.

FIG. 2. Milatuzumab does not alter CD86 expression on APC subsets, or IFN-γ primed, LPS-stimulated, IL-12 production by PBMCs. PBMCs were incubated with PBS, hMN-14, or milatuzumab, and stimulated with IFN-γ (100 ng/ml) for 18 h, followed by LPS (10 μg/ml) for an additional 24 h. The cells and the supernatants were collected for assessment of CD86 expression (FIG. 2A) and IL-12 production (FIG. 2B), respectively. The cells were stained with PE-conjugated anti-CD19 and anti-CD14, APC-conjugated anti-BDCA-1, and Alexa Fluor 488-conjugated anti-CD86 antibodies. B cells, monocytes, mDC1, and non-B lymphocytes were gated according to the same strategy as described in the legend to FIG. 1. Data are shown as the means±SD of the geo-mean fluorescence intensity of CD86 expression in different cell subsets, in triplicates from two donors. The IL-12 concentration in the supernatants was measured by ELISA, and the data are shown as the means±SD of the OD_{450 nm} in triplicates from two donors.

FIG. 3. Milatuzumab reduces T-cell proliferation in allo-MLR. CFSE-labeled PBMCs from two different donors were mixed and incubated with different antibodies at 5 μg/ml for 11 days, and the cells were harvested and analyzed by flow cytometry. The proliferating cells were quantitated by measuring the CFSE^{low} cell frequencies. Representative data from one combination of stimulator/responder PBMCs are shown in (FIG. 3A), and the statistical analysis of all combinations is shown in (FIG. 3B). Error bars, SD, n=10 stimulator/responder combinations. **, P<0.05; and ***P<0.01 vs. hMN-14. ##, P<0.05 vs. hLL1.

FIG. 4. Anti-HLA antibody IMMU-114 depletes all subsets of human PBMCs. Human PBMCs were incubated with 5 μg/ml IMMU-114, control antibodies (hMN-14 and rituximab), or medium only, for 3 days. The effect of each treatment on APC subsets was evaluated by co-staining the cells with PE-labeled anti-CD14 and anti-CD19, in combination with APC-labeled anti-BDCA-1 or anti-BDCA-2, for analysis of mDC1 and pDCs, respectively; or a mixture of FITC-labeled anti-BDCA-2 and APC-labeled anti-BDCA-3 for analysis of mDC2. 7-AAD was added before flow cytometric analyses. PBMCs were gated to exclude debris and dead cells on the basis of their forward and side scatter characteristics. The subpopulations of PBMCs were gated as follows: monocytes, CD14⁺SSC^{medium}; B cells, CD19⁺SSC^{low}; non-B lymphocytes (mostly T cells), CD19⁻CD14⁻SSC^{low}; mDC1, CD14⁻CD19⁻BDCA-1⁺. The live cell fraction of each cell population was determined by measuring 7-AAD^{neg} cells. Mean percentages of live mDC1, mDC2, B cells, monocytes, and non-B lymphocytes in PBMCs, relative to untreated control (Medium), are shown (n=6-7 donors). Error bars, SD; **, P<0.01 vs. hMN-14.

FIG. 5. IMMU-114 is cytotoxic to purified mDC1, mDC2, or pDCs. mDC1, mDC2, and pDCs were isolated from human PBMCs using magnetic beads, and treated for 2 days with IMMU-114 or control antibody hMN-14, followed by 7-AAD staining for flow cytometry analysis of cell viability of mDC1 (FIG. 5A), pDCs (FIG. 5B), and mDC2 (FIG. 5C). The numbers represent the percentages of live cells in the acquired total events. Data shown are representative of 2 donors.

FIG. 6. IMMU-114 reduces T-cell proliferation in allo-MLR cultures. CFSE-labeled PBMCs from two different donors were mixed and incubated with IMMU-114 or control antibody hMN-14 at 5 μg/ml for 11 days, and the cells were harvested and analyzed by flow cytometry. The proliferating cells were quantitated by measuring the CFSE^{low} cell frequencies. The statistical analysis of all combinations of stimulator/responder PBMCs is shown. Error bars, SD, n=10 stimulator/responder combinations from 5 donors. **P<0.01 vs. hMN-14.

### DETAILED DESCRIPTION

Definitions

As used herein, the terms “a”, “an” and “the” may refer to either the singular or plural, unless the context otherwise makes clear that only the singular is meant.

An “antibody” refers to a full-length (i.e., naturally occurring or formed by normal immunoglobulin gene fragment recombinatorial processes) immunoglobulin molecule (e.g., an IgG antibody) or an immunologically active (i.e., antigen-binding) portion of an immunoglobulin molecule, like an antibody fragment.

An “antibody fragment” is a portion of an antibody such as F(ab′)₂, F(ab)₂, Fab′, Fab, Fv, scFv, single domain antibodies (DABS or VHHs) and the like, including half-molecules of IgG4 (van der Neut Kolfschoten et al. (Science 2007; 317(14 September):1554-1557). Regardless of structure, an antibody fragment binds with the same antigen that is recognized by the intact antibody. For example, an anti-CD74 antibody fragment binds with an epitope of CD74. The term “antibody fragment” also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”).

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains. Additional BR amino acid substitutions from the parent, e.g. murine, antibody may be made. The constant domains of the antibody molecule are derived from those of a human antibody.

A “human antibody” is, for example, an antibody obtained from transgenic mice that have been genetically engineered to produce human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. (See, e.g., McCafferty et al., Nature 348:552-553 (1990) for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

A “therapeutic agent” is an atom, molecule, or compound that is useful in the treatment of a disease. Examples of therapeutic agents include but are not limited to antibodies, antibody fragments, drugs, toxins, enzymes, nucleases, hormones, immunomodulators, antisense oligonucleotides, chelators, boron compounds, photoactive agents, dyes and radioisotopes.

A “diagnostic agent” is an atom, molecule, or compound that is useful in diagnosing a disease. Useful diagnostic agents include, but are not limited to, radioisotopes, dyes, contrast agents, fluorescent compounds or molecules and enhancing agents (e.g., paramagnetic ions). Preferably, the diagnostic agents are selected from the group consisting of radioisotopes, enhancing agents, and fluorescent compounds.

An “immunoconjugate” is a conjugate of an antibody, antibody fragment, antibody fusion protein, bispecific antibody or multispecific antibody with an atom, molecule, or a higher-ordered structure (e.g., with a carrier, a therapeutic agent, or a diagnostic agent). A “naked antibody” is an antibody that is not conjugated to any other agent.

As used herein, the term “antibody fusion protein” is a recombinantly produced antigen-binding molecule in which an antibody or antibody fragment is linked to another protein or peptide, such as the same or different antibody or antibody fragment or a DDD or AD peptide. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity. For example, a diabody, where one binding site reacts with one antigen and the other with another antigen.

A “bispecific antibody” is an antibody that can bind simultaneously to two targets which are of different structure. Bispecific antibodies (bsAb) and bispecific antibody fragments (bsFab) may have at least one arm that specifically binds to, for example, an APC and/or DC antigen or epitope and at least one other arm that binds to a different antigen or epitope. The second arm may bind to a different APC or DC antigen or it may bind to a targetable conjugate that bears a therapeutic or diagnostic agent. A variety of bispecific antibodies can be produced using molecular engineering.

Anti-CD74 and Anti-HLA-DR Antibodies

CD74

The CD74 antigen is an epitope of the major histocompatibility complex (MHC) class II antigen invariant chain, Ii, present on the cell surface and taken up in large amounts of up to 8×10⁶ molecules per cell per day (Hansen et al., 1996, Biochem. J., 320: 293-300). CD74 is present on the cell surface of B-lymphocytes, monocytes and histocytes, human B-lymphoma cell lines, melanomas, T-cell lymphomas and a variety of other tumor cell types. (Hansen et al., 1996, Biochem. J., 320: 293-300) CD74 associates with α/β chain MHC II heterodimers to form MHC II αβIi complexes that are involved in antigen processing and presentation to T cells (Dixon et al., 2006, Biochemistry 45:5228-34; Loss et al., 1993, J Immunol 150:3187-97; Cresswell et al., 1996; Cell 84:505-7).

CD74 plays an important role in cell proliferation and survival. Binding of the CD74 ligand, macrophage migration inhibitory factor (MIF), to CD74 activates the MAP kinase cascade and promotes cell proliferation (Leng et al., 2003, J Exp Med 197:1467-76). Binding of MIF to CD74 also enhances cell survival through activation of NF-κB and Bcl-2 (Lantner et al., 2007, Blood 110:4303-11).

The Examples below demonstrate that milatuzumab (hLL1), a humanized anti-CD74 antibody, can selectively and significantly deplete myeloid DC type 1 (mDC1) and type 2 (mDC2), mildly but significantly depletes B cells, but has little effect on plasmacytoid DCs (pDCs), monocytes, or T cells within human peripheral blood mononuclear cells (PBMCs). The depleting efficiency was correlated with CD74 expression levels of each cell type. Killing of mDC1 and mDC2 by milatuzumab was by an Fc-mediated mechanism, as evidenced by the lack of effect of hLL1-Fab-A3B3, a fusion protein of the Fab of milatuzumab linked to an irrelevant protein domain, and by the failure of milatuzumab to kill purified mDC1 or mDC2 in the absence of PBMCs. Milatuzumab suppressed allogenic T-cell proliferation in mixed leukocyte cultures, but preserved CMV-specific CD8⁺ T cells.

HLA-DR

The human leukocyte antigen-DR (HLA-DR) is one of three polymorphic isotypes of the class II major histocompatibility complex (MHC) antigen. Because HLA-DR is expressed at high levels on a range of hematologic malignancies, there has been considerable interest in its development as a target for antibody-based lymphoma therapy. However, safety concerns have been raised regarding the clinical use of HLA-DR-directed antibodies, because the antigen is expressed on normal as well as tumor cells. (Dechant et al., 2003, Semin Oncol 30:465-75) HLA-DR is constitutively expressed on normal B cells, monocytes/macrophages, dendritic cells, and thymic epithelial cells. In addition, interferon-gamma may induce HLA class II expression on other cell types, including activated T and endothelial cells (Dechant et al., 2003).

The most widely recognized function of HLA molecules is the presentation of antigen in the form of short peptides to the antigen receptor of T lymphocytes. In addition, signals delivered via HLA-DR molecules contribute to the functioning of the immune system by up-regulating the activity of adhesion molecules, inducing T-cell antigen counterreceptors, and initiating the synthesis of cytokines. (Nagy and Mooney, 2003, J Mol Med 81:757-65; Scholl et al., 1994, Immunol Today 15:418-22)

As disclosed in the Examples below, humanized anti-HLA-DR antibody, IMMU-114 or hL243i4P (Stein et al. Blood 108:2736-2744, 2006), can deplete all subsets of APCs, but not T cells, from human peripheral blood mononuclear cells (PBMCs), including myeloid DCs (mDCs), plasmacytoid DCs (pDCs), B cells, and monocytes. In the absence of other human cells or complement, purified mDCs or pDCs were still killed efficiently by IMMU-114, suggesting that IMMU-114 depletes these APCs in PBMCs independently of antibody-dependent cellular cytotoxicity (ADCC) or complement-dependent cytotoxicity (CDC). Furthermore, IMMU-114 suppressed the proliferation of allo-reactive T cells in mixed leukocyte cultures, yet preserved CMV-specific, CD8⁺ memory T cells. Together, these results support the use of IMMU-114 as a novel conditioning regimen for maximally preventing aGVHD without altering preexisting anti-viral immunity.

Although the Examples below demonstrate the use of milatuzumab as an exemplary anti-CD74 antibody and IMMU-114 as an exemplary anti-HLA-DR antibody, the skilled artisan will realize that other anti-CD74 and/or anti-HLA-DR antibodies known in the art may be utilized in the claimed methods and compositions.

Preparation of Antibodies

The immunoconjugates and compositions described herein may include monoclonal antibodies. Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)).

General techniques for cloning murine immunoglobulin variable domains have been disclosed, for example, by the publication of Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), disclose how they produced an LL2 chimera by combining DNA sequences encoding the Vₖ and V_{H} domains of LL2 monoclonal antibody, an anti-CD22 antibody, with respective human and IgG₁ constant region domains. This publication also provides the nucleotide sequences of the LL2 light and heavy chain variable regions, Vₖ and V_{H}, respectively. Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

A chimeric antibody is a recombinant protein that contains the variable domains including the CDRs derived from one species of animal, such as a rodent antibody, while the remainder of the antibody molecule; i.e., the constant domains, is derived from a human antibody. Accordingly, a chimeric monoclonal antibody can also be humanized by replacing the sequences of the murine FR in the variable domains of the chimeric antibody with one or more different human FR. Specifically, mouse CDRs are transferred from heavy and light variable chains of the mouse immunoglobulin into the corresponding variable domains of a human antibody. As simply transferring mouse CDRs into human FRs often results in a reduction or even loss of antibody affinity, additional modification might be required in order to restore the original affinity of the murine antibody. This can be accomplished by the replacement of one or more some human residues in the FR regions with their murine counterparts to obtain an antibody that possesses good binding affinity to its epitope. (See, e.g., Tempest et al., Biotechnology 9:266 (1991) and Verhoeyen et al., Science 239: 1534 (1988)).

A fully human antibody can be obtained from a transgenic non-human animal. (See, e.g., Mendez et al., Nature Genetics, 15: 146-156, 1997; U.S. Pat. No. 5,633,425.) Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as an immune dysfunction disease (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.) Recombinant Fab were cloned from the μ, γ and κ chain antibody repertoires and inserted into a phage display library (Id.) RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J. Immunol. Methods 231:11-23, incorporated herein by reference) from Abgenix (Fremont, Calif.). In the XENOMOUSE® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. A XENOMOUSE® immunized with a target antigen will produce human antibodies by the normal immune response, which may be harvested and/or produced by standard techniques discussed above. A variety of strains of XENOMOUSE® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

Known Antibodies

In various embodiments, the claimed methods and compositions may utilize any of a variety of antibodies known in the art. Antibodies of use may be commercially obtained from a number of known sources. For example, a variety of antibody secreting hybridoma lines are available from the American Type Culture Collection (ATCC, Manassas, Va.). A large number of antibodies against various disease targets have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572;856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206′ 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953, 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art.

Exemplary known antibodies include, but are not limited to, hPAM4 (U.S. Pat. No. 7,282,567), hA20 (U.S. Pat. No. 7,251,164), hA19 (U.S. Pat. No. 7,109,304), hIMMU31 (U.S. Pat. No. 7,300,655), hLL1 (U.S. Pat. No. 7,312,318,), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. Provisional Patent Application 61/145,896), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/13 (WO 2009/130575). Other known antibodies are disclosed, for example, in U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20040202666 (now abandoned); 20050271671; and 20060193865. The text of each recited patent or application is incorporated herein by reference with respect to the Figures and Examples sections.

Antibody Fragments

Antibody fragments which recognize specific epitopes can be generated by known techniques. The antibody fragments are antigen binding portions of an antibody, such as F(ab)₂, Fab′, Fab, Fv, scFv and the like. Other antibody fragments include, but are not limited to, F(ab′)₂ fragments which can be produced by pepsin digestion of the antibody molecule and Fab′ fragments which can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are disclosed in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIB′I′ECH, Vol 9: 132-137 (1991).

An antibody fragment can be prepared by known methods, for example, as disclosed by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL.1, page 422 (Academic Press 1967), and Coligan at pages 2.8.1-2.8.10 and 2.10.-2.10.4.

A single complementarity-determining region (CDR) is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. (See, e.g., Larrick et al., Methods: A Companion to Methods in Enzymology 2: 106 (1991); Courtenay-Luck, “Genetic Manipulation of Monoclonal Antibodies,” in MONOCLONAL ANTIBODIES: PRODUCTION, ENGINEERING AND CLINICAL APPLICATION, Ritter et al. (eds.), pages 166-179 (Cambridge University Press 1995); and Ward et al., “Genetic Manipulation and Expression of Antibodies,” in MONOCLONAL ANTIBODIES: PRINCIPLES AND APPLICATIONS, Birch et al., (eds.), pages 137-185 (Wiley-Liss, Inc. 1995).

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780).

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797).

Multispecific and Multivalent Antibodies

Various embodiments may concern use of multispecific and/or multivalent antibodies. For example, an anti-CD74 antibody or fragment thereof and an anti-HLA-DR antibody or fragment thereof may be joined together by means such as the dock-and-lock technique described below. Other combinations of antibodies or fragments thereof may be utilized. For example, the anti-CD74 or anti-HLA-DR antibody could be combined with another antibody against a different epitope of the same antigen, or alternatively with an antibody against another antigen expressed by the APC or DC cell, such as CD209 (DC-SIGN), CD34, CD74, CD205, TLR 2 (toll-like receptor 2), TLR 4, TLR 7, TLR 9, BDCA-2, BDCA-3, BDCA-4 or HLA-DR.

Methods for producing bispecific antibodies include engineered recombinant antibodies which have additional cysteine residues so that they crosslink more strongly than the more common immunoglobulin isotypes. (See, e.g., FitzGerald et al, Protein Eng 10:1221-1225, 1997). Another approach is to engineer recombinant fusion proteins linking two or more different single-chain antibody or antibody fragment segments with the needed dual specificities. (See, e.g., Coloma et al., Nature Biotech. 15:159-163, 1997). A variety of bispecific antibodies can be produced using molecular engineering. In one form, the bispecific antibody may consist of, for example, a scFv with a single binding site for one antigen and a Fab fragment with a single binding site for a second antigen. In another form, the bispecific antibody may consist of, for example, an IgG with two binding sites for one antigen and two scFv with two binding sites for a second antigen.

Diabodies, Triabodies and Tetrabodies

The compositions disclosed herein may also include functional bispecific single-chain antibodies (bscAb), also called diabodies. (See, e.g., Mack et al., Proc. Natl. Acad. Sci., 92: 7021-7025, 1995). For example, bscAb are produced by joining two single-chain Fv fragments via a glycine-serine linker using recombinant methods. The V light-chain (V_{L}) and V heavy-chain (V_{H}) domains of two antibodies of interest are isolated using standard PCR methods. The V_{L} and V_{H} cDNAs obtained from each hybridoma are then joined to form a single-chain fragment in a two-step fusion PCR. The first PCR step introduces the linker, and the second step joins the V_{L} and V_{H} amplicons. Each single chain molecule is then cloned into a bacterial expression vector. Following amplification, one of the single-chain molecules is excised and sub-cloned into the other vector, containing the second single-chain molecule of interest. The resulting bscAb fragment is subcloned into a eukaryotic expression vector. Functional protein expression can be obtained by transfecting the vector into Chinese Hamster Ovary cells.

For example, a humanized, chimeric or human anti-CD74 and/or anti-HLA-DR monoclonal antibody can be used to produce antigen specific diabodies, triabodies, and tetrabodies. The monospecific diabodies, triabodies, and tetrabodies bind selectively to targeted antigens and as the number of binding sites on the molecule increases, the affinity for the target cell increases and a longer residence time is observed at the desired location. For diabodies, the two chains comprising the V_{H} polypeptide of the humanized CD74 or HLA-DR antibody connected to the V_{K} polypeptide of the humanized CD74 or HLA-DR antibody by a five amino acid residue linker may be utilized. Each chain forms one half of the diabody. In the case of triabodies, the three chains comprising V_{H} polypeptide of the humanized CD74 or HLA-DR antibody connected to the V_{K} polypeptide of the humanized CD74 or HLA-DR antibody by no linker may be utilized. Each chain forms one third of the triabody.

More recently, a tetravalent tandem diabody (termed tandab) with dual specificity has also been reported (Cochlovius et al., Cancer Research (2000) 60: 4336-4341). The bispecific tandab is a dimer of two identical polypeptides, each containing four variable domains of two different antibodies (V_{H1}, V_{L1}, V_{H2}, V_{L2}) linked in an orientation to facilitate the formation of two potential binding sites for each of the two different specificities upon self-association.

Dock-and-Lock (DNL)

In certain preferred embodiments, bispecific or multispecific antibodies may be produced using the dock-and-lock (DNL) technology (see, e.g., U.S. Pat. Nos. 7,521,056; 7,550,143; 7,534,866; 7,527,787 and 7,666,400; the Examples section of each of which is incorporated herein by reference). The DNL method exploits specific protein/protein interactions that occur between the regulatory (R) subunits of cAMP-dependent protein kinase (PKA) and the anchoring domain (AD) of A-kinase anchoring proteins (AKAPs) (Baillie et al., FEBS Letters. 2005; 579: 3264. Wong and Scott, Nat. Rev. Mol. Cell Biol. 2004; 5: 959). PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968;243:3763). The structure of the holoenzyme consists of two catalytic subunits held in an inactive form by the R subunits (Taylor, J. Biol. Chem. 1989;264:8443). Isozymes of PKA are found with two types of R subunits (RI and RID, and each type has α and β isoforms (Scott, Pharmacol. Ther. 1991;50:123). Thus, there are four types of PKA regulatory subunits—RIα, RIβ, RIIα and RIIβ. The R subunits have been isolated only as stable dimers and the dimerization domain has been shown to consist of the first 44 amino-terminal residues (Newlon et al., Nat. Struct. Biol. 1999; 6:222). Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990;265;21561).

Since the first AKAP, microtubule-associated protein-2, was characterized in 1984 (Lohmann et al., Proc. Natl. Acad. Sci USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004;5:959). The AD of AKAPs for PKA is an amphipathic helix of 14-18 residues (Carr et al., J. Biol. Chem. 1991;266:14188). The amino acid sequences of the AD are quite varied among individual AKAPs, with the binding affinities reported for RII dimers ranging from 2 to 90 nM (Alto et al., Proc. Natl. Acad. Sci. USA. 2003;100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIα are both located within the same N-terminal 44 amino acid sequence (Newlon et al., Nat. Struct. Biol. 1999;6:222; Newlon et al., EMBO J. 2001;20:1651), which is termed the DDD herein.

We have developed a platform technology to utilize the DDD of human PKA regulatory subunit and the AD of AKAP as an excellent pair of linker modules for docking any two entities, referred to hereafter as A and B, into a noncovalent complex, which could be further locked into a stably tethered structure through the introduction of cysteine residues into both the DDD and AD at strategic positions to facilitate the formation of disulfide bonds. The general methodology of the “dock-and-lock” approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001;98:8480) to ligate site-specifically. Using various combinations of linkers, adaptor modules and precursors, a wide variety of DNL constructs of different stoichiometry may be produced and used, including but not limited to dimeric, trimeric, tetrameric, pentameric and hexameric DNL constructs (see, e.g., U.S. Pat. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

The skilled artisan will realize that the DNL technique may be utilized to produce complexes comprising multiple copies of the same anti-CD74 or anti-HLA-DR antibody, or to attach one or more anti-CD74 antibodies to one or more anti-HLA-DR antibodies, or to attach an anti-HLA-DR or anti-CD74 antibody to an antibody that binds to a different antigen expressed by APCs and/or DCs. Alternatively, the DNL technique may be used to attach antibodies to different effector moieties, such as toxins, cytokines, carrier proteins for siRNA and other known effectors.

Amino Acid Substitutions

In various embodiments, the disclosed methods and compositions may involve production and use of proteins or peptides with one or more substituted amino acid residues. For example, the DDD and/or AD sequences used to make DNL constructs may be modified as discussed below.

The skilled artisan will be aware that, in general, amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL website at rockefeller.edu) For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

Pre-Targeting

In certain alternative embodiments, therapeutic agents may be administered by a pretargeting method, utilizing bispecific or multispecific antibodies. In pretargeting, the bispecific or multispecific antibody comprises at least one binding arm that binds to an antigen exhibited by a targeted cell or tissue, such as CD74 or HLA-DR, while at least one other binding arm binds to a hapten on a targetable construct. The targetable construct comprises one or more haptens and one or more therapeutic and/or diagnostic agents.

Pre-targeting is a multistep process originally developed to resolve the slow blood clearance of directly targeting antibodies, which contributes to undesirable toxicity to normal tissues such as bone marrow. With pre-targeting, a radionuclide or other diagnostic or therapeutic agent is attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

Immunoconjugates

In preferred embodiments, an antibody or antibody fragment may be directly attached to one or more therapeutic agents to form an immunoconjugate. Therapeutic agents may be attached, for example to reduced SH groups and/or to carbohydrate side chains. A therapeutic agent can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the therapeutic agent can be conjugated via a carbohydrate moiety in the Fc region of the antibody.

Methods for conjugating functional groups to antibodies via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, the Examples section of which is incorporated herein by reference. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); U.S. Pat. Nos. 5,443,953 and 6,254,868, the Examples section of which is incorporated herein by reference. The engineered carbohydrate moiety is used to attach the therapeutic or diagnostic agent.

An alternative method for attaching therapeutic agents to a targeting molecule involves use of click chemistry reactions. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tomoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. Although the copper catalyst is toxic to living cells, the copper-based click chemistry reaction may be used in vitro for immunoconjugate formation.

A copper-free click reaction has been proposed for covalent modification of biomolecules. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions (Id.)

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.) Examples of such substituted cyclooctynes include difluorinated cyclooctynes, 4-dibenzocyclooctynol and azacyclooctyne (Id.) An alternative copper-free reaction involved strain-promoted akyne-nitrone cycloaddition to give N-alkylated isoxazolines (Id.) The reaction was reported to have exceptionally fast reaction kinetics and was used in a one-pot three-step protocol for site-specific modification of peptides and proteins (Id.) Nitrones were prepared by the condensation of appropriate aldehydes with N-methylhydroxylamine and the cycloaddition reaction took place in a mixture of acetonitrile and water (Id.) These and other known click chemistry reactions may be used to attach therapeutic agents to antibodies in vitro.

The specificity of the click chemistry reaction may be used as a substitute for the antibody-hapten binding interaction used in pretargeting with bispecific antibodies. In this alternative embodiment, the specific reactivity of e.g., cyclooctyne moieties for azide moieties or alkyne moieties for nitrone moieties may be used in an in vivo cycloaddition reaction. An antibody or other targeting molecule is activated by incorporation of a substituted cyclooctyne, an azide or a nitrone moiety. A targetable construct is labeled with one or more diagnostic or therapeutic agents and a complementary reactive moiety. I.e., where the targeting molecule comprises a cyclooctyne, the targetable construct will comprise an azide; where the targeting molecule comprises a nitrone, the targetable construct will comprise an alkyne, etc. The activated targeting molecule is administered to a subject and allowed to localize to a targeted cell, tissue or pathogen, as disclosed for pretargeting protocols. The reactive labeled targetable construct is then administered. Because the cyclooctyne, nitrone or azide on the targetable construct is unreactive with endogenous biomolecules and highly reactive with the complementary moiety on the targeting molecule, the specificity of the binding interaction results in the highly specific binding of the targetable construct to the tissue-localized targeting molecule.

Therapeutic Agents

A wide variety of therapeutic reagents can be administered concurrently or sequentially with the anti-CD74 and/or anti-HLA-DR antibodies. For example, drugs, toxins, oligonucleotides, immunomodulators, hormones, hormone antagonists, enzymes, enzyme inhibitors, radionuclides, angiogenesis inhibitors, other antibodies or fragments thereof, etc. The therapeutic agents recited here are those agents that also are useful for administration separately with an antibody or fragment thereof as described above. Therapeutic agents include, for example, cytotoxic agents such as vinca alkaloids, anthracyclines, gemcitabine, epipodophyllotoxins, taxanes, antimetabolites, alkylating agents, antibiotics, SN-38, COX-2 inhibitors, antimitotics, anti-angiogenic and pro-apoptotic agents, particularly doxorubicin, methotrexate, taxol, CPT-11, camptothecans, proteosome inhibitors, mTOR inhibitors, HDAC inhibitors, tyrosine kinase inhibitors, and others.

Other useful cytotoxic agents include nitrogen mustards, alkyl sulfonates, nitrosoureas, triazenes, folic acid analogs, COX-2 inhibitors, antimetabolites, pyrimidine analogs, purine analogs, platinum coordination complexes, mTOR inhibitors, tyrosine kinase inhibitors, proteosome inhibitors, HDAC inhibitors, camptothecins, hormones, and the like. Suitable cytotoxic agents are described in REMINGTON'S PHARMACEUTICAL SCIENCES, 19th Ed. (Mack Publishing Co. 1995), and in GOODMAN AND GILMAN′S THE PHARMACOLOGICAL BASIS OF THERAPEUTICS, 7th Ed. (MacMillan Publishing Co. 1985), as well as revised editions of these publications.

In a preferred embodiment, conjugates of camptothecins and related compounds, such as SN-38, may be conjugated to an anti-CD74 or anti-HLA-DR antibody, for example as disclosed in U.S. Pat. No. 7,591,994, the Examples section of which is incorporated herein by reference.

A toxin can be of animal, plant or microbial origin. A toxin, such as Pseudomonas exotoxin, may also be complexed to or form the therapeutic agent portion of an immunoconjugate. Other toxins include ricin, abrin, ribonuclease (RNase), DNase I, Staphylococcal enterotoxin-A, pokeweed antiviral protein, onconase, gelonin, diphtheria toxin, Pseudomonas exotoxin, and Pseudomonas endotoxin. See, for example, Pastan et al., Cell 47:641 (1986), Goldenberg, CA—A Cancer Journal for Clinicians 44:43 (1994), Sharkey and Goldenberg, CA—A Cancer Journal for Clinicians 56:226 (2006). Additional toxins suitable for use are known to those of skill in the art and are disclosed in U.S. Pat. No. 6,077,499, the Examples section of which is incorporated herein by reference.

As used herein, the term “immunomodulator” includes cytokines, lymphokines, monokines, stem cell growth factors, lymphotoxins, hematopoietic factors, colony stimulating factors (CSF), interferons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-α, TGF-β, insulin-like growth factor (IGF), erythropoietin, thrombopoietin, tumor necrosis factor (TNF), TNF-α, TNF-β, mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-α, interferon-β, interferon-γ, S1 factor, IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21, IL-25, LIF, kit-ligand, FLT-3, angiostatin, thrombospondin, endostatin, LT, and the like.

The antibody or fragment thereof may be administered as an immunoconjugate comprising one or more radioactive isotopes useful for treating diseased tissue. Particularly useful therapeutic radionuclides include, but are not limited to ¹¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, and ²¹¹Pb. The therapeutic radionuclide preferably has a decay energy in the range of 20 to 6,000 keV, preferably in the ranges 60 to 200 keV for an Auger emitter, 100-2,500 keV for a beta emitter and 4,000-6,000 keV for an alpha emitter. Maximum decay energies of useful beta-particle-emitting nuclides are preferably 20-5,000 keV, more preferably 100-4,000 keV and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2,000-10,000 keV, more preferably 3,000-8,000 keV, and most preferably 4,000-7,000 keV.

Additional potential therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵_{Tm,} ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

Interference RNA

In certain preferred embodiments the therapeutic agent may be a siRNA or interference RNA species. The siRNA, interference RNA or therapeutic gene may be attached to a carrier moiety that is conjugated to an antibody or fragment thereof. A variety of carrier moieties for siRNA have been reported and any such known carrier may be incorporated into a therapeutic antibody for use. Non-limiting examples of carriers include protamine (Rossi, 2005, Nat Biotech 23:682-84; Song et al., 2005, Nat Biotech 23:709-17); dendrimers such as PAMAM dendrimers (Pan et al., 2007, Cancer Res. 67:8156-8163); polyethylenimine (Schiffelers et al., 2004, Nucl Acids Res 32:e149); polypropyleneimine (Taratula et al., 2009, J Control Release 140:284-93); polylysine (Inoue et al., 2008, J Control Release 126:59-66); histidine-containing reducible polycations (Stevenson et al., 2008, J Control Release 130:46-56); histone H1 protein (Haberland et al., 2009, Mol Biol Rep 26:1083-93); cationic comb-type copolymers (Sato et al., 2007, J Control Release 122:209-16); polymeric micelles (U.S. Patent Application Publ. No. 20100121043); and chitosan-thiamine pyrophosphate (Rojanarata et al., 2008, Pharm Res 25:2807-14). The skilled artisan will realize that in general, polycationic proteins or polymers are of use as siRNA carriers. The skilled artisan will further realize that siRNA carriers can also be used to carry other oligonucleotide or nucleic acid species, such as anti-sense oligonucleotides or short DNA genes.

Known siRNA species of potential use include those specific for IKK-gamma (U.S. Pat. No. 7,022,828); VEGF, Flt-1 and Flk-1/KDR (U.S. Pat. No. 7,148,342); Bc12 and EGFR (U.S. Pat. No. 7,541,453); CDC20 (U.S. Pat. No. 7,550,572); transducin (beta)-like 3 (U.S. Pat. No. 7,576,196); K-ras (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Mirus Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL complexes.

Exemplary siRNA species known in the art are listed in Table 1. Although siRNA is delivered as a double-stranded molecule, for simplicity only the sense strand sequences are shown in Table 1.

TABLE 1
Exemplary siRNA Sequences

| Target | Sequence | SEQ ID NO |
|---|---|---|
| VEGF R2 | AATGCGGCGGTGGTGACAGTA | SEQ ID NO: 13 |
| VEGF R2 | AAGCTCAGCACACAGAAAGAC | SEQ ID NO: 14 |
| CXCR4 | UAAAAUCUUCCUGCCCACCdTdT | SEQ ID NO: 15 |
| CXCR4 | GGAAGCUGUUGGCUGAAAAdTdT | SEQ ID NO: 16 |
| PPARC1 | AAGACCAGCCUCUUUGCCCAG | SEQ ID NO: 17 |
| Dynamin 2 | GGACCAGGCAGAAAACGAG | SEQ ID NO: 18 |
| Catenin | CUAUCAGGAUGACGCGG | SEQ ID NO: 19 |
| E1A binding protein | UGACACAGGCAGGCUUGACUU | SEQ ID NO: 20 |
| Plasminogen | GGTGAAGAAGGGCGTCCAA | SEQ ID NO: 21 |
| activator | | |
| K-ras | GATCCGTTGGAGCTGTTGGCGTAGTT | SEQ ID NO: 22 |
| | CAAGAGACTCGCCAACAGCTCCAACT | |
| | TTTGGAAA | |
| Sortilin 1 | AGGTGGTGTTAACAGCAGAG | SEQ ID NO: 23 |
| Apolipoprotein E | AAGGTGGAGCAAGCGGTGGAG | SEQ ID NO: 24 |
| Apolipoprotein E | AAGGAGTTGAAGGCCGACAAA | SEQ ID NO: 25 |
| Bcl-X | UAUGGAGCUGCAGAGGAUGdTdT | SEQ ID NO: 26 |
| Raf-1 | TTTGAATATCTGTGCTGAGAACACA | SEQ ID NO: 27 |
| | GTTCTCAGCACAGATATTCTTTTT | |
| Heat shock | AATGAGAAAAGCAAAAGGTGCCCTGTCTC | SEQ ID NO: 28 |
| transcription factor 2 | | |
| IGFBP3 | AAUCAUCAUCAAGAAAGGGCA | SEQ ID NO: 29 |
| Thioredoxin | AUGACUGUCAGGAUGUUGCdTdT | SEQ ID NO: 30 |
| CD44 | GAACGAAUCCUGAAGACAUCU | SEQ ID NO: 31 |
| MMP14 | AAGCCTGGCTACAGCAATATGCCTGTCTC | SEQ ID NO: 32 |
| MAPKAPK2 | UGACCAUCACCGAGUUUAUdTdT | SEQ ID NO: 33 |
| FGFR1 | AAGTCGGACGCAACAGAGAAA | SEQ ID NO: 34 |
| ERBB2 | CUACCUUUCUACGGACGUGdTdT | SEQ ID NO: 35 |
| BCL2L1 | CTGCCTAAGGCGGATTTGAAT | SEQ ID NO: 36 |
| ABL1 | TTAUUCCUUCUUCGGGAAGUC | SEQ ID NO: 37 |
| CEACAM1 | AACCTTCTGGAACCCGCCCAC | SEQ ID NO: 38 |
| CD9 | GAGCATCTTCGAGCAAGAA | SEQ ID NO: 39 |
| CD151 | CATGTGGCACCGTTTGCCT | SEQ ID NO: 40 |
| Caspase 8 | AACTACCAGAAAGGTATACCT | SEQ ID NO: 41 |
| BRCA1 | UCACAGUGUCCUUUAUGUAdTdT | SEQ ID NO: 42 |
| p53 | GCAUGAACCGGAGGCCCAUTT | SEQ ID NO: 43 |
| CEACAM6 | CCGGACAGTTCCATGTATA | SEQ ID NO: 44 |

The skilled artisan will realize that Table 1 represents a very small sampling of the total number of siRNA species known in the art, and that any such known siRNA may be utilized in the claimed methods and compositions.

Methods of Therapeutic Treatment

The claimed methods and compositions are of use for treating disease states, such as autoimmune disease or immune system dysfunction (e.g., aGVHD). The methods may comprise administering a therapeutically effective amount of a therapeutic antibody or fragment thereof or an immunoconjugate, either alone or in conjunction with one or more other therapeutic agents, administered either concurrently or sequentially.

Multimodal therapies may include therapy with other antibodies, such as anti-CD209 (DC-SIGN), anti-CD34, anti-CD74, anti-CD205, anti-TLR-2, anti-TLR-4, anti- TLR-7, anti-TLR-9, anti-BDCA-2, anti- BDCA-3, anti- BDCA-4 or anti-HLA-DR (including the invariant chain) antibodies in the form of naked antibodies, fusion proteins, or as immunoconjugates. Various antibodies of use are known to those of skill in the art. See, for example, Ghetie et al., Cancer Res. 48:2610 (1988); Hekman et al., Cancer Immunol. Immunother. 32:364 (1991); Longo, Curr. Opin. Oncol. 8:353 (1996), U.S. Pat. Nos. 5,798,554; 6,187,287; 6,306,393; 6,676,924; 7,109,304; 7,151,164; 7,230,084; 7,230,085; 7,238,785; 7,238,786; 7,282,567; 7,300,655; 7,312,318; 7,612,180; 7,501,498; the Examples section of each of which is incorporated herein by reference.

In another form of multimodal therapy, subjects receive therapeutic antibodies in conjunction with standard chemotherapy. For example, cyclophosphamide, etoposide, carmustine, vincristine, procarbazine, prednisone, doxorubicin, methotrexate, bleomycin, dexamethasone or leucovorin, alone or in combination. Additional useful drugs include phenyl butyrate, bendamustine, and bryostatin-1. In a preferred multimodal therapy, both cytotoxic drugs and cytokines are co-administered with a therapeutic antibody. The cytokines, cytotoxic drugs and therapeutic antibody can be administered in any order, or together.

Therapeutic antibodies or fragments thereof can be formulated according to known methods to prepare pharmaceutically useful compositions, whereby the therapeutic antibody is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, EMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The therapeutic antibody can be formulated for intravenous administration via, for example, bolus injection or continuous infusion. Preferably, the therapeutic antibody is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The therapeutic antibody may also be administered to a mammal subcutaneously or even by other parenteral routes. Moreover, the administration may be by continuous infusion or by single or multiple boluses. Preferably, the therapeutic antibody is infused over a period of less than about 4 hours, and more preferably, over a period of less than about 3 hours.

More generally, the dosage of an administered therapeutic antibody for humans will vary depending upon such factors as the patient's age, weight, height, sex, general medical condition and previous medical history. It may be desirable to provide the recipient with a dosage of therapeutic antibody that is in the range of from about 1 mg/kg to 25 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy.

Alternatively, a therapeutic antibody may be administered as one dosage every 2 or 3 weeks, repeated for a total of at least 3 dosages. Or, the therapeutic antibody may be administered twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 20 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

Additional pharmaceutical methods may be employed to control the duration of action of the therapeutic immunoconjugate or naked antibody. Control release preparations can be prepared through the use of polymers to complex or adsorb the immunoconjugate or naked antibody. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release of an immunoconjugate or antibody from such a matrix depends upon the molecular weight of the immunoconjugate or antibody, the amount of immunoconjugate or antibody within the matrix, and the size of dispersed particles. Saltzman et al., Biophys. J. 55: 163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Therapy of Autoimmune Disease

Anti-CD74 and/or anti-HLA-DR antibodies or immunoconjugates can be used to treat immune dysregulation disease and related autoimmune diseases. Immune diseases may include acute idiopathic thrombocytopenic purpura, Addison's disease, adult respiratory distress syndrome (ARDS), agranulocytosis, allergic conditions, allergic encephalomyelitis, allergic neuritis, amyotrophic lateral sclerosis (ALS), ankylosing spondylitis, antigen-antibody complex mediated diseases, anti-glomerular basement membrane disease, anti-phospholipid antibody syndrome, aplastic anemia, arthritis, asthma, atherosclerosis, autoimmune disease of the testis and ovary, autoimmune endocrine diseases, autoimmune myocarditis, autoimmune neutropenia, autoimmune polyendocrinopathies, autoimmune polyglandular syndromes (or polyglandular endocrinopathy syndromes), autoimmune thrombocytopenia, Bechet disease, Berger's disease (IgA nephropathy), bronchiolitis obliterans (non-transplant), bullous pemphigoid, Castleman's syndrome, Celiac sprue (gluten enteropathy), central nervous system (CNS) inflammatory disorders, chronic active hepatitis, chronic idiopathic thrombocytopenic purpura dermatomyositis, colitis, conditions involving infiltration of T cells and chronic inflammatory responses, coronary artery disease, Crohn's disease, cryoglobulinemia, dermatitis, dermatomyositis, diabetes mellitus, diseases involving leukocyte diapedesis, eczema, encephalitis, erythema multiforme, erythema nodosum, Factor VIII deficiency, fibrosing alveolitis, giant cell arteritis, glomerulonephritis, Goodpasture's syndrome, graft versus host disease (GVHD), granulomatosis, Grave's disease, Guillain-Barre Syndrome, Hashimoto's thyroiditis, hemophilia A, Henoch-Schonlein purpura, idiopathic hypothyroidism, idiopathic thrombocytopenic purpura (ITP), IgA nephropathy, IgA nephropathy, IgM mediated neuropathy, immune complex nephritis, immune hemolytic anemia including autoimmune hemolytic anemia (AIHA), immune responses associated with acute and delayed hypersensitivity mediated by cytokines and T-Iymphocytes, immune-mediated thrombocytopenias, juvenile onset diabetes, juvenile rheumatoid arthritis, Lambert-Eaton Myasthenic Syndrome, large vessel vasculitis, leukocyte adhesion deficiency, leukopenia, lupus nephritis, lymphoid interstitial pneumonitis (HIV), medium vessel vasculitis, membranous nephropathy, meningitis, multiple organ injury syndrome, multiple sclerosis, myasthenia gravis, osteoarthritis, pancytopenia, pemphigoid bullous, pemphigus vulgaris, pernicious anemia, polyarteritis nodosa, polychondritis, polyglandular syndromes, polymyalgia, polymyositis, post-streptococcal nephritis, primary biliary cirrhosis, primary hypothyroidism, psoriasis, psoriatic arthritis, pure red cell aplasia (PRCA), rapidly progressive glomerulonephritis, Reiter's disease, respiratory distress syndrome, responses associated with inflammatory bowel disease, Reynaud's syndrome, rheumatic fever, rheumatoid arthritis, sarcoidosis, scleroderina, Sjogren's syndrome, solid organ transplant rejection, Stevens-Johnson syndrome, stiff-man syndrome, subacute thyroiditis, Sydenham's chorea, systemic lupus erythematosus (SLE), systemic scleroderma and sclerosis, tabes dorsalis, Takayasu's arteritis, thromboangitis obliterans, thrombotic thrombocytopenic purpura (TTP), thyrotoxicosis, toxic epidermal necrolysis, tuberculosis, Type I diabetes , ulcerative colitis, uveitis, vasculitis (including ANCA) and Wegener's granulomatosis. In a particularly preferred embodiment, the disease to be treated is aGVHD.

### EXAMPLES

Various embodiments of the present invention are illustrated by the following examples, without limiting the scope thereof.

### Example 1

### Depletion of Human Myeloid Dendritic Cells by Anti-CD74 Antibody for Control of Graft-Versus-Host Disease

CD74 (invariant chain, Ii) is a type-II transmembrane glycoprotein that associates with the major histocompatibility class (MHC) II α and β chains and directs the transport of the Pali complexes to endosomes and lysosomes. The proinflammatory cytokine, macrophage migration-inhibitory factor (MIF), binds to cell surface CD74, initiating a signaling cascade involving activation of NF-κB. CD74 is expressed by certain normal HLA class II-positive cells, including B cells, monocytes, macrophages, Langerhans cells, dendritic cells, subsets of activated T cells, and thymic epithelium. CD74 is also expressed on a variety of malignant cells, including the vast majority of B-cell cancers (NHL, CLL, MM).

The LL1 monoclonal antibody was generated by hybridoma technology after immunization of BALB/c mice with Raji human Burkitt lymphoma cells. The LL1 antibody reacts with an epitope in the extracellular domain of CD74. CD74-positive cell lines have been shown to very rapidly internalize LL1 (nearly 10⁷ molecules per cell per day). This rapid internalization enables LL1 to be an extremely effective agent for delivery of cytotoxic agents, such as chemotherapeutics or toxins.

Previous studies have shown that milatuzumab (humanized anti-CD74 LL1 antibody), in the presence of an anti-human IgG Fc antibody, shows potent in vitro cytotoxicity against CD74-expressing malignant B-cell lines, including non-Hodgkin lymphoma (NHL) and multiple myeloma (MM), and exhibits therapeutic efficacy in vivo in xenografted NHL and MM malignancies (Stein et al., 2004, Blood 104:3705-3711; Stein et al., 2007, Clin Cancer Res. 13:5556s-5563s; Burton et al., 2004, Clin Cancer Res. 10:6606-6611; Stein et al., 2009, Clin Cancer Res. 15:2808-2817). Currently, milatuzumab is under clinical evaluation as a therapeutic antibody for relapsed or refractory B-cell malignancies (Berkova et al., 2010, Expert Opin Investig Drugs 19:141-149).

In addition to expression on malignant B cells, CD74 is also present in normal APCs, such as B cells, monocytes, macrophages, Langerhans cells, and follicular and blood DCs (Stein et al., 2007, Clin Cancer Res. 13:5556s-5563s; Freudenthal & Steinman, 1990, Proc Natl Acad Sci U S A 87:7698-7702). We have previously reported that exposure of human whole blood to milatuzumab has little effect on the viability of B cells and T cells (Stein et al., 2010, Blood 115:5180-90). However, it has not been determined previously whether milatuzumab has any effects on the viability of mDC1, pDCs, mDC2, and monocytes. The present Example assessed the binding profile and cytotoxicity of milatuzumab on all APC subsets of human PBMCs, including mDC1, pDCs, mDC2, B cells, T cells, and monocytes. As shown below, exposure of PBMCs to milatuzumab caused potent depletion of mDC1 and mDC2, mild depletion of B cells, and no effect on pDCs, monocytes, and T cells, which could be correlated with CD74 expression levels on these cells. These results distinguish milatuzumab from T-cell antibodies and support use of milatuzumab for preventing and treating GVHD.

Materials and Methods

Antibodies and reagents—Milatuzumab (hLL1, U.S. Pat. No. 7,312,318), labetuzumab (hMN-14, U.S. Pat. No. 6,676,924), epratuzumab (hLL2, U.S. Pat. No. 7,074,403), and hLL1-Fab-A3B3 (U.S. Pat. No. 7,354,587), the Examples section of each cited patent incorporated herein by reference, were obtained as disclosed. Rituximab was purchased from IDEC Pharmaceuticals Corp. (San Diego, Calif.). Commercially available antibodies were obtained from BD Pharmingen (San Diego, Calif.): anti-CD86 (2331[FUN-1]), FITC-conjugated anti-CD74 (M-B741), and PerCP-conjugated anti-HLA-DR (L243 [G46-6]) and CD3 (SK7); or from Miltenyi Biotec (Auburn, Calif.): PE-conjugated antibodies to CD19 (LT19) and CD14 (TUK4), and allophycocyanin (APC)-conjugated antibodies to BDCA-1 (AD5-8E7), BDCA-2 (AC144), and BDCA-3 (AD5-14H12). Milatuzumab and anti-CD86 were labeled with the ZENON® ALEXA FLUOR® 488 human IgG labeling kit (Invitrogen, Carlsbad, Calif.) following the manufacturer's instructions.

Purification of myeloid and plasmacytoid DCs and NK/Non-NK cells from PBMCs—PBMCs were isolated from the buffy coats of healthy donors by standard density-gradient centrifugation over FICOLL-PAQUE™ (Lonza, Walkersville, Md.). mDC1 were purified from PBMCs by depleting CD19⁺ B cells, followed by positive enrichment of BDCA-1⁺ cells. pDCs were purified by depleting all the cells that do not express BDCA-4 antigen. mDC2 were purified by enriching BDCA-3⁺ cells. The BDCA-3⁻ cells that contained no mDC2 were used for isolation of NK cells by depleting all the cells that do not express CD56. Those depleted cells that contained neither NK cells nor mDC2 were used as non-NK cells. All the purification procedures were performed according to the manual of MACS® kits (Miltenyi Biotec).

Ex-vivo depletion of APC subsets in PBMC—PBMCs from normal donors were treated with milatuzumab or other antibodies at 37° C., 5% CO₂, for 3 days. Following incubation, the cells were stained with PE-labeled anti-CD14 and anti-CD19, in combination with APC-labeled anti-BDCA-1. After washing, 7-amino-actinomycin D (7-AAD, BD Pharmingen) was added, and the cells were analyzed by flow cytometry using the gating strategy described below. The live PBMCs were gated based on the forward scatter (FSC) and side scatter (SSC) signals. Within the live PBMCs, mDC1 were identified as CD14⁻19⁻BDCA-1⁺ cell populations (Morel et al., 2002, Immunology 106:229-236). Within the same live PBMCs, the lymphocyte population was analyzed for B cells (CD19⁺SSC^{low}), non-B lymphocytes (primarily T cells) (CD19⁻14⁻SSC^{low}), and monocytes (CD14⁺SSC^{medium}). The live cell fraction of each cell population was quantitated as the percentage of 7-AAD⁻ cells. To measure the frequencies of pDCs and mDC2, PBMCs were stained with PE-labeled anti-CD14 and anti-CD19, in combination with FITC-labeled anti-BDCA-2 and APC-labeled anti-BDCA-3. Within the live PBMCs, mDC2 were identified as CD14⁻19⁻BDCA-3⁺⁺ cell population, whereas pDCs were identified as CD14⁻19⁻BDCA-2⁺ cell population. Flow cytometry was performed using a FACSCALIBUR® (BD Bioscience) and analyzed with FlowJo software (Tree Star, Inc., Ashland, Oreg.).

Binding of anti-CD74 antibodies with human PBMC subsets—Human PBMCs isolated from buffy coats of healthy donors were treated with FcR-blocking reagent (Miltenyi Biotec), then co-stained with PE-conjugated antibodies to CD19 and CD14, FITC-labeled mouse anti-human CD74 antibody (M-B741), or its isotype control; or Alexa 488-conjugated milatuzumab, or human IgG control, and APC-conjugated antibody to BDCA-1, BDCA-2, or BDCA-3. The cells were washed and analyzed by flow cytometry. B cells and monocytes were gated according to the same FL2 signal (PE-labeled anti-CD14 and anti-CD19) combined with their differential SSC signals. The CD14⁻19⁻ cell populations were used to gate the BDCA-1⁺, BDCA-2⁺, or BDCA-3⁺ cell populations for mDC1, pDCs, and mDC2, respectively (Dzionek et al., 2000, J Immunol 165: 6037.-6046). The binding efficiency of milatuzumab or M-B741 with these cell populations was assessed by FL1 mean fluorescence intensity (MFI).

T-cell proliferation in allogeneic mixed leukocyte reaction—PBMCs from different donors were labeled with 1 μM carboxyfluorescein succinimidyl ester (CFSE) following the manufacturer's instructions (Invitrogen, Calif.). After extensive washings, the cells from two different donors were mixed and incubated for 11 days. The cells were then harvested and analyzed by flow cytometry. The proliferating cells were quantitated by measuring the CFSE^{low} cell frequencies (Han et al., 2008, Mol Ther. 16:269-279).

Assessment of CMV-specific IFN-γ response—PBMCs were prepared as described above. The cells were incubated with CMV pp65 15-mer overlapping peptides (PEPTIVATOR®, Miltenyi Biotec, Auburn, Calif.) or pp65 protein (Miltenyi Biotec) (Wills et al., 1996, J Virol 70:7569-7579; Tabi et al., 2001, J Immunol 166:5695-5703), and 2 h later, brefeldin A at 1 μg/ml final concentration was added. After 4 h of additional incubation, the cells were fixed and permeabilized by using BD CYTOFIX/CYTOPERM™ solution (BD Pharmingen), and analyzed by cell surface staining with PerCp-CD8 and intracellular staining with FITC-interferon-γ (IFN-γ) antibody. The percentages of IFN-γ⁺ cells stimulated by cytomegalovirus (CMV) pp65 peptides in both CD8⁺ and CD8⁻ T cells were assessed.

Quantitation of CMV-specific T cells in allo-MLR by HLA-A*0201 pentamer—PBMCs from a donor with a CMV-specific IFN-γ response were mixed with PBMCs from another donor, irrespective of his/her CMV status, in the presence of milatuzumab or control antibodies at 5 μg/ml. The mixed cells were cultured for 4 days in RPMI 1640 medium with 10% fetal bovine serum (FBS), followed by addition of 50 U/ml IL-2 and were further cultured for 2 more days. The cells were then harvested and stained with PE-labeled HLA-A*0201 CMV pentamer (Prolmmune, Bradenton, Fla.) (Wills et al., 1996, J Virol 70:7569-7579; Tabi et al., 2001, J Immunol 166:5695-5703), followed by washing and staining with PerCp-CD8 (BD Pharmingen). The percentages of CMV pentamer⁺ cells in CD8⁺ T cells were assessed by flow cytometry.

Statistical analysis—Statistical significance between antibody treatment and control was determined by paired t-test (Stein et al., 2010, Blood 115:5180-90). The Pearson correlation analysis was performed for regression of CD74 expression level and cell depletion.

Results

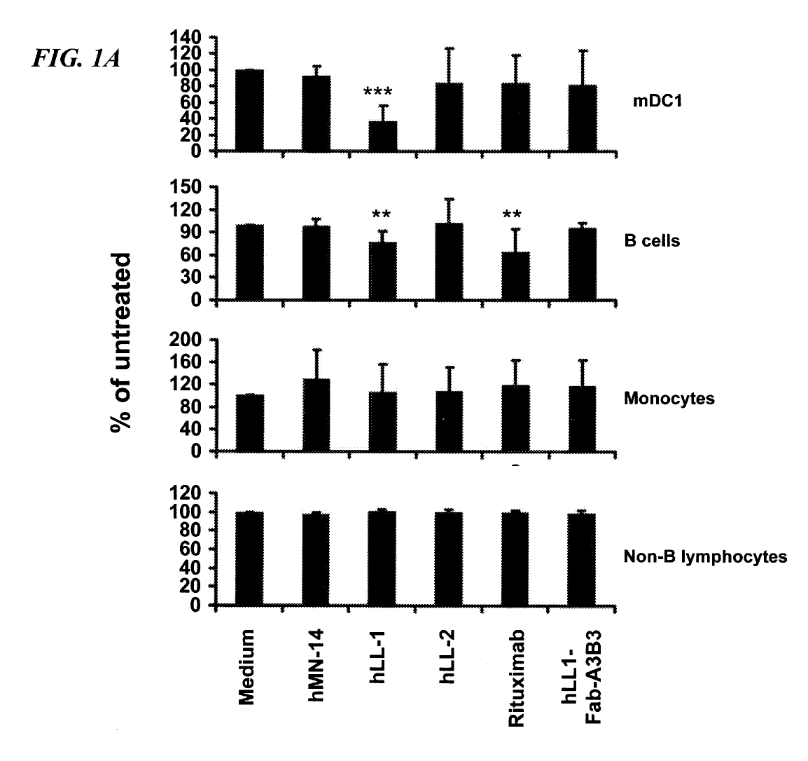

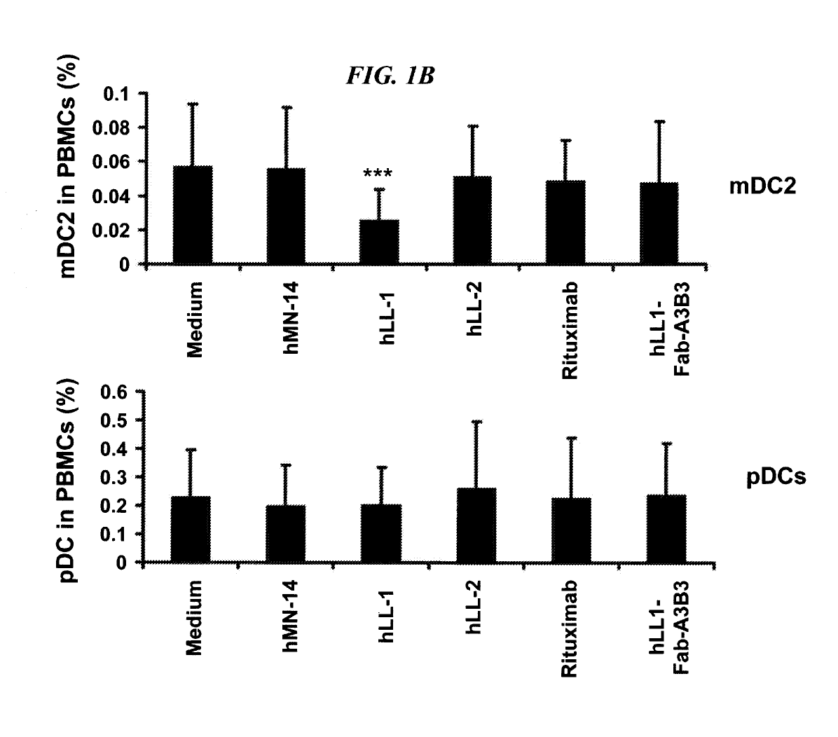

Milatuzumab selectively deplets myeloid DCs in human PBMCs—Milatuzumab is an antagonist antibody against CD74, which has been shown to have potent cytotoxicity against CD74-expressing B-cell lymphomas and multiple myeloma (Stein et al., 2004, Blood 104:3705-3711; Burton et al., 2004, Clin Cancer Res. 10:6606-6611; Stein et al., 2009, Clin Cancer Res. 15:2808-2817). Since most normal APCs or DCs express CD74 (Stein et al., 2007, Clin Cancer Res. 13:5556s-5563s; Freudenthal et al. 1990, Proc Natl Acad Sci USA, 87:7698-7702), milatuzumab may also be cytotoxic to these normal cells. We treated PBMCs with milatuzumab or other antibodies for 3 days, followed by an evaluation of the depletion of the various APC subsets in PBMCs. hMN-14 (humanized anti-CEACAM5), rituximab (chimeric anti-CD20), hLL2 (humanized anti-CD22, epratuzumab), and the Fc-lacking hLL1-Fab-A3B3, the Fab fragment of milatuzumab fused to the A3B3 domain of CEACAM5 (Hefta et al., 1992, Cancer Res. 52:5647-5655), were included for comparison. Of the antibodies evaluated, only milatuzumab significantly reduced the counts of live mDC1 and mDC2 in PBMCs. In three experiments, mDC1 in milatuzumab-treated PBMCs were reduced by 60.8% (P<.05, n=6 donors) (see FIG. 1A), 25.4% (P<0.05, n=7 donors), and 82% (P<0.05, n=4 donors), respectively. In one experiment, B cells were reduced by 22% (P=0.033), with no depletion (reduction <10%) in 2/6 donors, whereas monocytes and non-B lymphocytes (T and null cells) were little affected by milatuzumab (FIG. 1A). Rituximab significantly reduced B cells (by 36%, P=0.050, with no depletion of B cells (reduction <10%) in 1/6 donors) (FIG. 1A), but did not affect any of the other cell populations, including mDC1, monocytes, and non-B lymphocytes. All APC subsets tested were not altered by epratuzumab (FIG. 1A). In another experiment, mDC2 in milatuzumab-treated PBMCs were reduced by 53.8% (P<0.05, n=7 donors), whereas pDCs were not affected (FIG. 1B). Both mDC2 and pDCs were not affected by rituximab or epratuzumab (FIG. 1A). In other two experiments, pDCs were mildly reduced by milatuzumab but without statistical significance (data not shown). These results demonstrate that milatuzumab, but not other therapeutic antibodies tested, selectively depletes mDC1 and mDC2 in human PBMCs, and show that milatuzumab is of use for prophylactic or therapeutic control of GVHD, since either host or donor mDCs play a critical role in acute GVHD.

The levels of CD74 expression based on the MFI determined by flow cytometry were found to be higher for mDC2 (MFI=67.8) and mDC1 (MFI=59.0) than pDCs (MFI=29.5), B cells (MF22.7), monocytes (MFI=16.4), and non-B lymphocytes (MFI=1.6) (not shown). Thus, the more efficient depletion of mDC1 and mDC2 by milatuzumab may be due to their high level of CD74 expression. This depletion efficacy on APC subsets was significantly correlated with their CD74 expression (not shown).

Depletion of mDC1 and mDC2 by milatuzumab requires Fc—Despite the significant cytotoxicity of milatuzumab toward mDC1 and mDC2, these cells were not depleted by hLL1-Fab-A3B3 (FIG. 1A, FIG. 1B), which lacks the Fc portion of antibody. These data suggest that the depletion of mDC1 or mDC2 by milatuzumab may be through an Fc-mediated mechanism. To verify this, we treated purified mDC1 with milatuzumab for 2 days in the absence or presence of purified autologous NK cells or non-NK cells, which had been depleted of NK cells and mDC2, and should comprise monocytes, B cells, mDC1, pDCs, T cells, and NKT cells. Cytotoxicity was evaluated by 7-AAD staining and flow cytometry. Milatuzumab failed to kill purified mDC1 or mDC2 when used alone (data not shown). However, the cytotoxicity of milatuzumab on mDC1 was partially restored in the presence of added non-NK cells (viable mDC1 decreased by 38.2±8.7%, n=2 donors, P=0.155 compared to the hMN-14 isotype control) or NK cells (16.7±1.4%, P=0.0411, n=2 donors) (not shown). In both donors, the cytotoxicity of milatuzumab on mDC1 was greater in the presence of non-NK than NK cells (not shown). Because of the small number of mDC2 cells, restoration of milatuzumab toxicity on this cell population was only tested in the presence of added NK cells. Restoration of the cytotoxicity of milatuzumab on mDC2 was not observed in the presence of added NK cells (data not shown). These results suggest that milatuzumab acts through an Fc-mediated mechanism to deplete mDC1 and mDC2 in PBMCs, which may preferentially involve non-NK cell components for the killing.

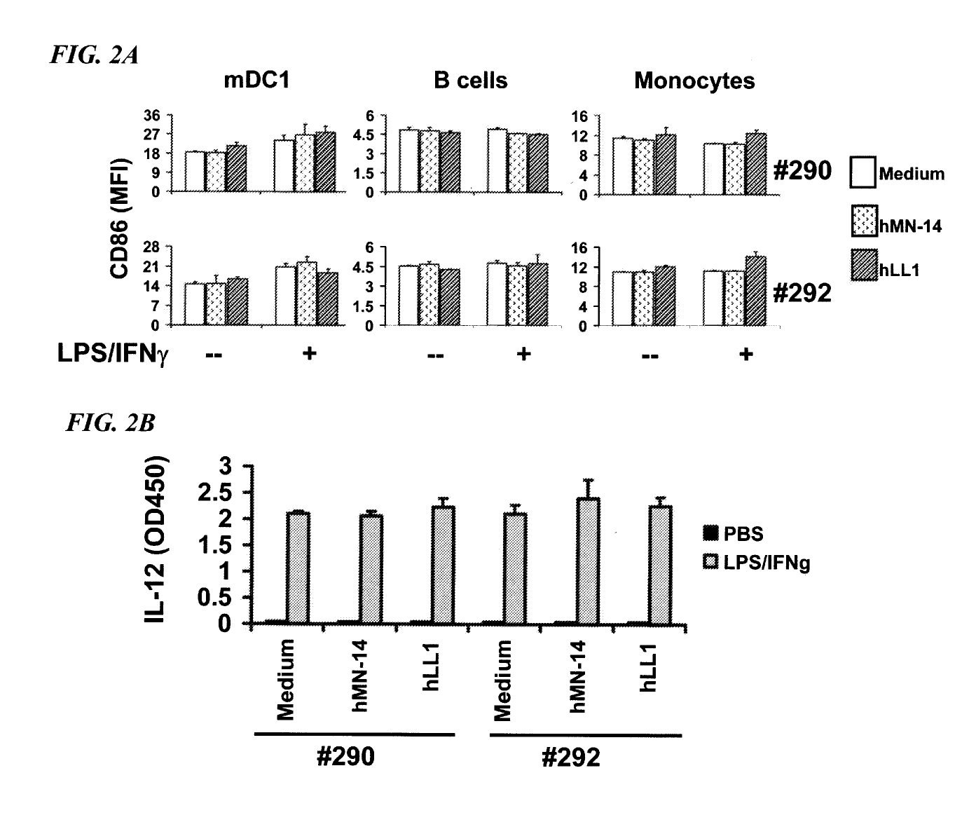

Milatuzumab does not affect CD86 expression and IL-12 production by human PBMCs—Because costimulatory molecules, including CD40, CD80 and CD86, are critical for donor APC function in intestinal and skin chronic GVHD (Anderson et al., 2005, Blood 105:2227-2234), we next investigated if milatuzumab had any effect on the expression of CD86 in mDC1, monocytes, B cells, and non-B lymphocytes. INF-γ□ and lipopolysaccharide (LPS) stimulate maturation of APCs and were included in this study to evaluate the effect of milatuzumab on both immature (without IFN-γ and LPS) and mature (with IFN-γ and LPS) cells. As shown in FIG. 2A, milatuzumab had little or no effect on CD86 expression in either mature or immature APCs.

IL-12, the “decisive” cytokine that drives type I immune response, may play a crucial role in the development of acute GVHD (Williamson et al., 1996, J Immunol 157:689-699; Yabe et al., 1999, Bone Marrow Transplant. 24:29-34). We therefore investigated if milatuzumab has any effect on IL-12 production by PBMCs upon stimulation by LPS/IFN-γ. As shown in FIG. 2B, milatuzumab had no effect on IL-12 production.

Thus, milatuzumab may not affect either “signal 2” (costimulatory molecules) or “signal 3” (cytokines) of APCs, suggesting that the antigen-presenting function of APCs is not affected by this antibody.

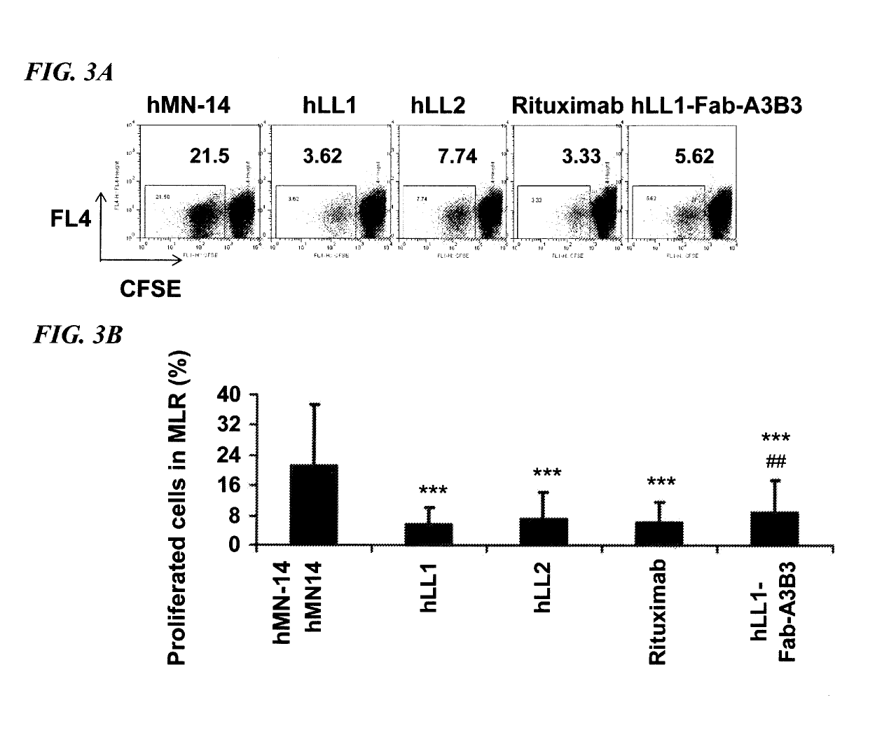

Milatuzumab reduces T-cell proliferation in allo-MLR—We next investigated whether the depletion of mDC1 and mDC2 in PBMCs by milatuzumab could be translated into reduced allo-proliferation of T cells. To do so, we mixed CFSE-labeled PBMCs from two different donors and maintained the cells in culture for 11 days in the presence of milatuzumab or control antibodies. The proliferated allo-reactive T cells were identified based on the CFSE dilution. As shown in FIG. 3A, the allo-MLR treated with the isotype control antibody, hMN-14, underwent robust T-cell proliferation characterized by 21.5% of T cells with CFSE dilution. In contrast, T-cell proliferation was only detected in 3.6% of cells in the MLR treated with milatuzumab. Statistical analysis of a total of 10 stimulator/responder combinations showed a significant reduction (P<0.01) in T-cell proliferation in milatuzumab-treated allo-MLR (FIG. 3B). Reduced allogeneic T-cell proliferation was also seen in rituximab-treated MLR (FIG. 3A, FIG. 3B). This may be due to the well-established cytotoxicity of rituximab on B cells (Reff et al., 1994, Blood 83:435-445). In summary, these data demonstrate a strong inhibitory effect of milatuzumab on allogeneic T-cell proliferation, suggesting that this novel antibody may have prophylactic and/or therapeutic potential for GVHD.

Preexisting anti-viral memory T cells are preserved in allo-MLR after milatuzumab treatment—As shown in FIG. 1, milatuzumab causes a potent depletion of mDC1s and mDC2s, but not non-B lymphocytes that are composed of mainly T cells. This is not unexpected, because the majority of T cells are resting cells, which lack the expression of CD74 (Stein et al., 2007, Clin Cancer Res 13:5556s-5563s). This result led us to reason that milatuzumab, while suppressing the proliferation of allo-reactive T cells, may preserve the preexisting pathogen-specific memory T cells. To confirm this, we first screened a panel of PBMC donors by measuring the CMV-specific IFN-γ response in CD8⁺ T cells stimulated in vitro by a CMV pp65 peptide pool. Of 4 donors tested, we identified one donor with a strong CMV-specific IFN-γ response, which HLA-typing revealed is HLA-A*0201 (data not shown). We then used this donor to determine whether CMV-specific T cells are preserved in allo-MLR after milatuzumab treatment. We first demonstrated that milatuzumab, even at a 10-fold higher concentration than was used for depletion of mDC1 and mDC2 (50 μg/ml), did not affect the CMV-specific IFN-γ response in CD8⁺ T cells stimulated in vitro by a CMV pp65 peptide pool or CMV pp65 protein (data not shown). A 6-day allo-MLR was then performed, in which the responder PBMCs were from this CMV-positive, HLA-A*0201 donor, and the stimulator PBMCs were from another donor, irrespective of CMV status. CMV-specific CD8⁺ T cells were determined by staining the cells with HLA*A0201 CMV pentamer (NLVPMVATV) (SEQ ID NO: 100). As expected, CMV-specific CD8⁺ T cells were not altered by milatuzumab treatment (not shown). This result is important, because CMV is one of the most prevalent pathogens that cause severe infections after allo-HSCT. The current standard immunosuppressive agents, such as high-dose steroids, effectively control GVHD but critically impair host immunity against pathogens. It is thus highly desired that any novel strategy against GVHD spare pathogen-specific immunity while suppressing the allo-specific response. Our results suggest that the third-party responses, such as pathogen-specific memory T-cell immunity, are not compromised by milatuzumab treatment.

Discussion

The critical role of DCs in the initiation of GVHD highlights the importance of DC depletion as a valuable approach to complement or replace current therapies for prophylactic and therapeutic control of GVHD. Depletion of DCs can be achieved by a number of antibodies. One example is the anti-CD52 antibody, alemtuzumab (Klangsinsirikul et al., 2002, Blood 99: 2586-2591; Ratzinger et al., 2003, Blood 101: 1422-1429), which has been used clinically for prevention of acute GVHD and is currently in clinical trials for the treatment of chronic GVHD. It can efficiently deplete host DCs and suppress the proliferation of allo-reactive T cells, but it also impairs anti-viral responses. RA83, a rabbit anti-human CD83 polyclonal antibody, is another DC-depleting agent, which targets activated DCs, leading to the suppression of allo-proliferation but without reducing CMV- or influenza-specific T cells (Munster et al., 2004, Int Immunol 16:33-42; Wilson et al., 2009, J Exp Med 206:387-398). However, use of rabbit polyclonal antibody for human therapy is likely to produce other undesirable side effects, such as immune response to the rabbit antibody.

In this study, we showed that milatuzumab, a humanized anti-CD74 antibody, can efficiently deplete myeloid DCs and suppress the proliferation of allo-reactive T cells, while preserving CMV-specific, CD8⁺ T cells. These findings show that anti-CD74 antibodies in general and milatuzumab in particular are novel DC-depleting antibodies for the control of GVHD. This can be used prophylactically to prevent acute GVHD, or therapeutically for chronic GVHD. In both cases, milatuzumab could offer the advantage of life-saving third-party immune functions being spared. This differs from current immunosuppressive therapies that suppress the overall immune functions without discrimination. This is very likely due to the lack of CD74 expression in T cells (Stein et al., 2007, Clin Cancer Res 13:5556s-5563s), with a corresponding lack of milatuzumab cytotoxicity on non-B lymphocytes (FIG. 1), which are mainly composed of T cells.

Another unique property is that milatuzumab selectively depleted mDCs, but not pDCs. It was reported that mouse donor CD11b⁻ pDCs could augment graft-versus-leukemic (GVL) activity without increasing GVHD (Li et al., 2009, J Immunol 183:7799-7809), suggesting that pDCs play an important role in GVL. The lack of effect on pDCs by milatuzumab suggests that it may not alter GVL activity while suppressing GVHD, which would be a favorable characteristic for GVHD control. In addition, pDCs are potentially tolerogenic in their immature status. It has been shown that CCR9-expressing pDCs are capable of suppressing GVHD (Hadeiba et al., 2008, Nat Immunol 9:1253-1260), supporting the idea that the sparing of pDCs by milatuzumab may be favorable in the control of GVHD.

Our results suggest that killing of mDC1 and mDC2 in PBMCs by milatuzumab is through an Fc-mediated mechanism, which preferentially involves non-NK cells, probably monocytes, for cytotoxicity. It has been reported that monocytes are the major contributor to mediate the in vivo B-cell depletion by anti-CD20 antibody (Uchida et al., 2004, J Exp Med. 199:1659-1669). The mechanism of milatuzumab on DCs may differ from that on malignant B cells, in which the cytotoxicity of milatuzumab is not through either ADCC or CDC, as revealed by a 4-h cytotoxicity assay, but through a direct inhibition of the NF-κB signaling pathway via blocking CD74 (Stein et al., 2009, Clin Cancer Res. 15:2808-2817; Stein et al., 2004, Blood 104:3705-3711; Binsky et al., 2007, Proc Natl Acad Sci USA 104:13408-13413). It may also differ from the CDC-dependent mechanism by which anti-CD52 antibody, alemtuzumab, depletes DCs (Klangsinsirikul et al., 2002, Blood 99:2586-2591).

In addition to DCs, other APCs, such as B cells, are also involved in the immunopathophysiology of acute and chronic GVHD (Shimabukuro-Vornhagen et al., 2009, Blood 114:4919-4927). Human B cells express CD20, CD22, and CD74, among other surface antigens. Our data demonstrate that rituximab, the chimeric anti-CD20 antibody, efficiently depletes B cells, whereas milatuzumab, the anti-CD74 antibody, only mildly depletes B cells, and epratuzumab (hLL2), the anti-CD22 antibody, does not show any cytotoxicity on B cells, yet does show a modest depletion of B cells clinically Domer et al., 2006, Arthritis Res Ther 8:R74). However, all these three antibodies effectively suppress the allo-reactive T-cell proliferation in MLR (FIG. 3), suggesting possible therapeutic value in GVHD.

The suppression of the allogeneic T-cell response by rituximab may be through both depletion and functional modification of B cells (Shimabukuro-Vornhagen et al., 2009, Blood 114:4919-4927). In the case of epratuzumab, it may regulate B-cell function to suppress the allo-response. Rituximab has been used clinically to effectively prevent acute GVHD and to treat chronic GVHD in allo-HSCT patients (Okamoto et al., 2006, Leukemia 20:172-173; Cutler et al., 2006, Blood 108:756-762). Although there is no report about the therapeutic effect on GVHD, epratuzumab has been shown to be effective in treating systemic lupus erythematosus patients Dömer & Goldenberg, 2007, Ther Clin Risk Manag 3:953-959; Jacobi et al., 2008, Ann Rheum Dis 67:450-457). It would be worthwhile to investigate the potential efficacy of epratuzumab in managing GVHD, as proposed by Shimabukuro-Vornhagen, et al. (2009, Blood 114:4919-4927). Milatuzumab, however, efficiently depletes myeloid DCs, the major and critical initiator of GVHD, and mildly but significantly depletes B cells, as well as downregulates CD19 expression on B cells (data not shown). It is thus expected that milatuzumab might be more potent in controlling GVHD than rituximab or epratuzumab.

In summary, we have shown that milatuzumab can selectively deplete myeloid DCs, the critical initiator of GVHD after allo-HSCT. Importantly, this antibody does not impair the anti-viral immune responses studied, while suppressing the allo-specific responses. Thus, it may be useful in patients with hematological malignancies or non-malignant diseases undergoing allogeneic HSCT. The outcome following allo-HSCT is expected to be improved by the control of GVHD by using this novel antibody to deplete host and donor myeloid dendritic cells.

### Example 2

### Depletion of All Antigen-Presenting Cells by Humanized Anti-HLA-DR Antibody Provides a Novel Conditioning Regimen With Maximal Protection Against GVHD

IMMU-114 is a humanized IgG4 anti-HLA-DR antibody derived from the murine anti-human HLA-DR antibody, L243. It recognizes a conformational epitope in the α-chain of HLA-DR (Stein et al., 2006, Blood 108:2736-2744). The engineered IgG4 isotype (hL243γ4P) of this humanized antibody abrogates its ADCC and CDC effector functions, but retains its antigen-binding properties and direct cytotoxicity against a variety of tumors (Stein et al., 2006, Blood 108:2736-2744), which is mediated through hyper-activation of ERK and JNK MAP kinase signaling pathways (Stein et al., 2010, Blood 115:5180-90).

Besides DCs, B cells and monocytes are the two other major subsets of circulating APCs. Accumulating evidence has demonstrated that B cells are involved in the pathogenesis of acute and chronic GVHD (Shimabukuro-Vornhagen et al., 2009, Blood 114:4919-4927) and that B-cell depleting therapy is effective in prevention and treatment of GVHD (Alousi et al., 2010, Leuk Lymphoma 51:376-389). The anti-CD20 antibody, rituximab, when included in the conditioning regimen, reduces the incidence of aGVHD (Christopeit et al., 2009, Blood 113:3130-3131). Monocytes may also be involved in the pathogenesis of GVHD, since higher numbers of blood monocytes before conditioning are associated with greater risk of aGVHD (Arpinati et al., 2007, Biol Blood Marrow Transplant 13:228-234). In addition, the proteosome inhibitor, bortezomib, which efficiently depletes monocytes (Arpinati et al., 2009, Bone Marrow Transplant 43:253-259), is active in controlling acute and chronic GVHD (Sun et al., 2004, Proc Natl. Acad Sci USA 101:8120-8125). Because each subset of APCs is involved in the pathogenesis of aGVHD, it is desirable to deplete all APC subsets during the preparative conditioning for allo-HSCT. This goal has not been attained by current regimens.

The results below show that the anti-HLA-DR antibody IMMU-114 or hL243γ4P can deplete all subsets of APCs, but not T cells, from human peripheral blood mononuclear cells (PBMCs), including myeloid DCs (mDCs), plasmacytoid DCs (pDCs), B cells and monocytes. In the absence of other human cells or complement, purified mDCs or pDCs were still killed efficiently by IMMU-114, suggesting that IMMU-114 depletes these APCs independently of antibody-dependent cellular cytotoxicity (ADCC) or complement-dependent cytotoxicity (CDC). Furthermore, IMMU-114 suppressed the proliferation of allo-reactive T cells in mixed leukocyte cultures, yet preserved CMV-specific, CD8⁺ memory T cells. Together, these results demonstrate the potential of IMMU-114 as a novel conditioning regimen for maximally preventing aGVHD without alteration of preexisting anti-viral immunity.

Methods

Antibodies—IMMU-114 (hL243γ4p, U.S. Pat. No. 7,612,180) and labetuzumab (hMN-14, U.S. Pat. No. 6,676,924) were prepared as described. Rituximab was purchased from IDEC Pharmaceuticals Corp. (San Diego, Calif.). Commercially available antibodies were obtained from Miltenyi Biotec (Auburn, Calif.):FITC-conjugated antibody to BDCA-2 (AC144), PE-conjugated antibodies to CD19 (LT19) and CD14 (TUK4), and allophycocyanin (APC)-conjugated antibodies to BDCA-1 (AD5-8E7), BDCA-2 (AC144), and BDCA-3 (AD5-14H12).

Purification of myeloid and plasmacytoid DCs from PBMCs—PBMCs were isolated from the buffy coats of healthy donors by standard density-gradient centrifugation over FICOLL-PAQUE™ (Lonza, Walkersville, Md.). MACS® kits (Miltenyi Biotec) were used to purify DC subsets from PBMCs. mDC1 cells were purified from PBMCs by depleting CD19⁺ B cells, followed by positive enrichment of BDCA-1⁺ cells. pDCs were purified by depleting all the cells that do not express BDCA-4 antigen. mDC2 cells were purified by enriching BDCA-3⁺ cells.

Flow cytometric analysis of APC subsets in human PBMCs—PBMCs from normal donors were treated with IMMU-114 or other antibodies at 37° C., 5% CO₂, for 3 days. Following incubation, the cells were stained with PE-labeled anti-CD14 and anti-CD19, in combination with APC-labeled anti-BDCA-1. After washing, 7-amino-actinomycin D (7-AAD, BD Pharmingen) was added, and the cells were analyzed by flow cytometry using the gating strategy described below. The live PBMCs were gated based on the forward scatter (FSC) and side scatter (SSC) signals. Within the live PBMCs, mDC1 cells were identified as CD14⁻19⁻BDCA-1⁺ cell populations (Dzionek et al., 2000, J Immunol 165:6037-6046). Within the same live PBMCs, the lymphocyte population was analyzed for B cells (CD19⁺SSC^{low}), non-B lymphocytes (primarily T cells) (CD19⁻14⁻SSC^{low}), and monocytes (CD14⁺SSC^{medium}). The live cell fraction of each cell population was quantitated as the percentage of 7-AAD⁻ cells. To measure the frequencies of pDCs and mDC2, PBMCs were stained with PE-labeled anti-CD14 and anti-CD19, in combination with FITC-labeled anti-BDCA-2 and APC-labeled anti-BDCA-3. Within the live PBMCs, mDC2 cells were identified as the CD14⁻19⁻BDCA-3⁺⁺ cell population, whereas pDCs were identified as the CD14⁻19⁻BDCA-2⁺ cell population. Flow cytometry was performed using a FACSCALIBUR® (BD Bioscience) and analyzed with FlowJo software (Tree Star, Inc., Ashland, Oreg.).

T-cell proliferation in allogeneic mixed leukocyte reaction—PBMCs from different donors were labeled with 1 μM carboxyfluorescein succinimidyl ester (CFSE) following the manufacturer's instructions (Invitrogen, Calif.). After extensive washings, the cells from two different donors were mixed and incubated for 11 days. The cells were then harvested and analyzed by flow cytometry. The proliferating cells were quantitated by measuring the CFSE^{low} cell frequencies.

Quantitation of CMV-specific T cells in allo-MLR by HLA-A*0201 pentamer—PBMCs from a donor with a CMV-specific IFN-γ response were mixed with PBMCs from another donor, irrespective of his/her CMV status, in the presence of IMMU-114 or control antibody hMN-14 at 5 μg/ml. The mixed cells were cultured for 4 days in RPMI 1640 medium with 10% fetal bovine serum (FBS), followed by addition of 50 U/ml IL-2 and were further cultured for 2 more days. The cells were then harvested and stained with PE-labeled HLA-A*0201 CMV pentamer (ProImmune, Bradenton, Fla.) (Wills et al., 1996, J Virol 70:7569-7579; Pita-Lopez et al., 2009, Immun. Ageing 6:11), followed by washing and staining with PerCp-CD8 (BD Pharmingen). The percentages of CMV pentamer⁺ cells in CD8⁺ T cells were assessed by flow cytometry.

Statistical analysis—Paired t-test was used to determine the P values comparing the effects between IMMU-114 and control antibody treatment.

Results

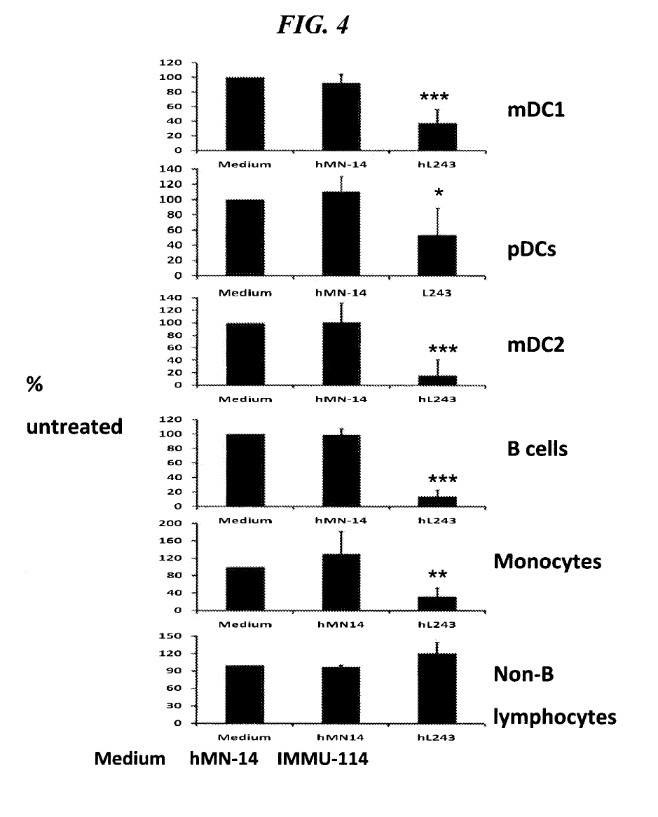

We have demonstrated previously that IMMU-114 efficiently depletes B cells and monocytes, but not T cells or NK cells from human whole blood in vitro (Stein et al., 2010, Blood 115:5180-90). Since both mDCs and pDCs express HLA-DR, IMMU-114 may also deplete these two major subsets of blood DCs. To investigate this, we treated human PBMCs with IMMU-114 or a control antibody (hMN-14 or labetuzumab, humanized anti-CEACAM5 antibody) (Sharkey et al., 1995, Cancer Res. 55(suppl):5935s-5945s) for 3 days, followed by quantitation of various APC subsets in PBMCs by flow cytometry. IMMU-114, but not hMN-14, depleted B cells and monocytes, but not non-B lymphocytes (the majority are T cells) (data not shown), which is consistent with our previous findings in whole blood samples (Stein et al., 2010, Blood 115:5180-90). All blood DC subsets in human PBMCs, including mDC type 1 (mDC1, the major subset of blood mDCs, Dzionek et al., 2000, J Immunol 165:6037-6046), pDCs, and mDC type 2 (mDC2, the minor subset of mDCs, Dzionek et al., 2000, J Immunol 165:6037-6046), were greatly reduced (not shown). As shown in FIG. 4, mDC1 were reduced by 59.2% (P=0.0022, n=6 donors), mDC2 by ˜85% (P<0.01, n=7 donors), B cells by 86.2% (P<0.001, n=6 donors), and monocytes by 74.7% (P=0.01139, n=6 donors), whereas non-B lymphocytes were not reduced. These results demonstrate that IMMU-114 can deplete all APC subsets in human PBMCs, and show that IMMU-114 may be used as a nonmyeloablative conditioning component to prevent aGVHD by maximum depletion of host APCs.

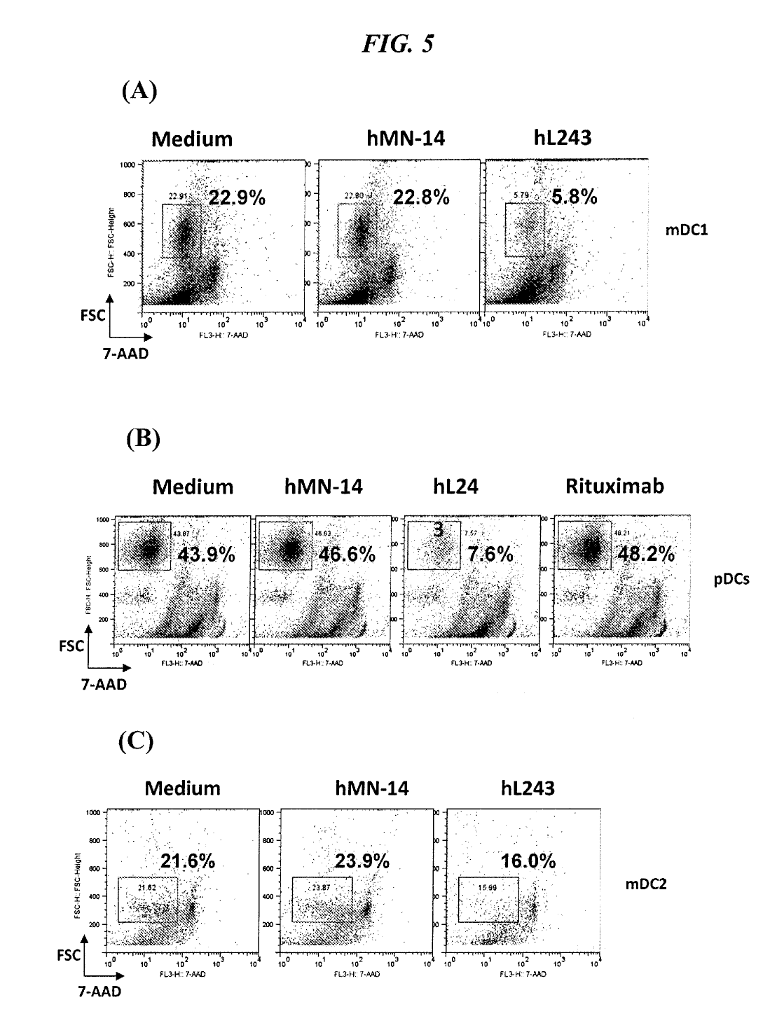

We next determined whether the depletion of APC subsets by IMMU-114 is direct. We isolated mDC1, mDC2, and pDCs from human PBMCs by MACS® selection and treated these purified cells for 2 days with IMMU-114 or control antibody, in the absence of any other cell types or human complement. Cytotoxicity was evaluated by 7-AAD staining and flow cytometry (Klangsinsirikul et al., 2002, Blood 99:2586-2591). In the absence of PBMCs or any other cells, IMMU-114 could still efficiently kill purified mDC1 (FIG. 5A), pDCs (FIG. 5B), or mDC2 (FIG. 5C). These results suggest that IMMU-114 exerts its cytotoxicity on APC subsets through direct action, independent of ADCC or CDC mechanisms.

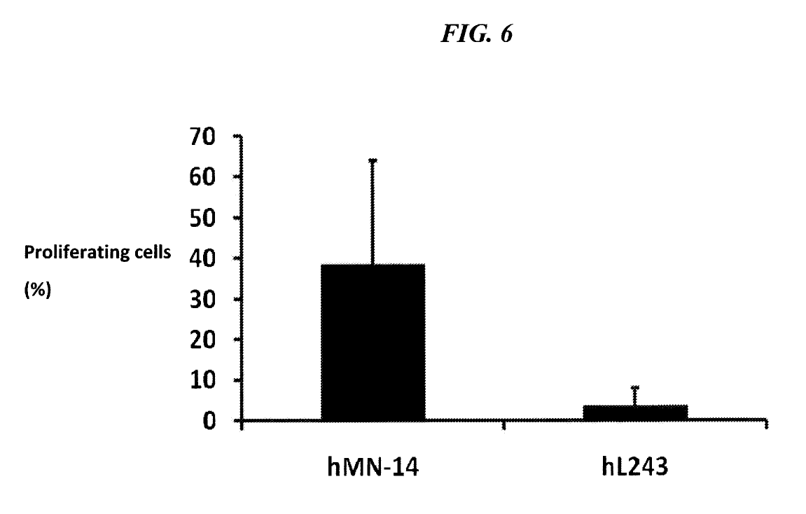

Since proliferation of allo-reactive T cells is a hallmark of GVHD (Wilson et al., 2009, J Exp Med 206:387-398), we investigated if the depletion of all APC subsets in PBMCs by IMMU-114 could be translated into reduced allo-proliferation of T cells. We mixed CFSE-labeled PBMCs from two different donors and maintained the cells in culture for 11 days in the presence of IMMU-114 or control antibody, hMN-14. The proliferating allo-reactive T cells were identified based on the CFSE dilution. The allo-MLR treated with the isotype control antibody, hMN-14 (anti-CEACAM5), underwent robust T-cell proliferation characterized by ˜50% of T cells with CFSE dilution. In contrast, T-cell proliferation was only detected in ˜5% of cells in the allo-MLR treated with IMMU-114 (not shown). Statistical analysis of a total of 10 stimulator/responder combinations showed a significant reduction (P<0.01) in T-cell proliferation in IMMU-114-treated allo-MLR (FIG. 6). These data demonstrate a strong inhibitory effect of IMMU-114 on allogeneic T-cell proliferation, indicating that introducing this novel antibody into the conditioning regimen will result in a prophylactic prevention potential against GVHD.

Alemtuzumab has been used extensively as a component of the conditioning regimen in patients undergoing allo-HSCT and has been demonstrated to significantly reduce GVHD (Kottaridis et al., 2000, Blood 96:2419-2425). However, alemtuzumab depletes both DCs and T cells, accounting for the increased reactivation of CMV in allo-HSCT patients (Perez-Simon et al., 2002, Blood 100:3121-3127; Chakrabarti et al., 2002, Blood 99:4357-4363). IMMU-114, however, does not affect T cells while depleting all subsets of APCs (FIG. 4). This unique property suggests that IMMU-114 does not affect CMV-specific memory T cells. To verify this, we performed a 6-day allo-MLR culture, in which the responder PBMCs were from a CMV-positive, HLA-A*0201 donor, and the stimulator PBMCs were from another donor, irrespective of CMV status. CMV-specific CD8⁺ T cells were determined by staining the cells with HLA*A0201 CMV pentamer. As expected, CMV-specific CD8⁺ T cells were not altered by IMMU-114 treatment (not shown). This result shows that pathogen-specific memory T-cell immunity, such as CMV-specific memory T cells, is not compromised by IMMU-114 treatment.

The results above obtained with samples from four donors showed that hL243 reduced pDCs by about 50%, but the decrease was not statistically significant (P=0.1927). PBMCs from six additional donors were further tested for the effect of hL243 or other antibodies on the survival of pDCs and the HLA-DR⁺pDC subset. hL243, but not hLL1, depleted plasmacytoid DCs in human PBMCs (data not shown). Human PBMCs were incubated with different mAbs or control at 5 μg/ml, in the absence or presence of GM-CSF (280 U/ml) and IL-3 (5 ng/ml). 3 days later, the cells were stained with APC-labeled BDCA-2 antibody and PerCp-labeled HLA-DR antibody. pDCs were defined as BDCA-2+ cells. hL243 (P=0.0114) but not hLL1 (P=0.5789) or other control antibodies produced a statistically significant decrease in pDCs (BDCA-2⁺) in the absence of GM-CSF and IL-3 (not shown). hL243 (P=0.0066) but not hLL1 (P=0.4799) or other control antibodies produced a statistically significant decrease in HLA-DR⁺ pDCs in the absence of GM-CSF and IL-3 (not shown). Neither hL243 (P=0.1250) nor hLL1 (P=0.2506) or other control antibodies produced a statistically significant decrease in pDCs in the presence of GM-CSF and IL-3 (not shown). hL243 (P=0.0695) but not hLL1 (P=0.2018) or other control antibodies produced a statistically significant decrease in HLA-DR⁺pDCs in the presence of GM-CSF and IL-3 (not shown). These results show that hL243, but not hLL1, depletes total pDCs and HLA-DR positive pDCs in human PBMCs. The depletion effects were antagonized by the presence of DC survival cytokines GM-CSF and IL-3.

Conclusions

We have shown that IMMU-114, a humanized anti-HLA-DR IgG4 antibody, can deplete all subsets of APCs efficiently, including mDC1, pDC, mDC2, B cells, and monocytes, leading to potent suppression of allo-reactive T cell proliferation, yet preserves CMV-specific, CD8⁺ memory T cells. These findings show that IMMU-114 could be a novel component of the conditioning regimen for allo-HSCT by depletion of all subsets of APCs. In comparison with currently-used alemtuzumab, IMMU-114 exhibits a number of surprising advantages. It depletes all APC subsets, providing maximal depletion of host APCs, whereas alemtuzumab depletes only peripheral blood DCs (Buggins et al., 2002, Blood 100:1715-1720). IMMU-114 does not affect T cells, leading to the preservation of pathogen-specific memory T cells, whereas alemtuzumab depletes T cells, leading to reactivation of CMV in allo-HSCT patients. IMMU-114 depletes APC subsets through direct action without the requirement of intact host immunity, whereas alemtuzumab depletes DCs through CDC- and ADCC-mediated mechanisms, which require intact host immune effector functions. Pharmacokinetic data in dogs indicate that IMMU-114 is rapidly cleared from the blood within several hours, followed by the clearance of remaining antibody with a half-life of ˜2 days (not shown), from which the half-life of IMMU-114 in humans is predicted to be 2-3 days according to the allometric scaling of an immunoglobulin fusion protein described by Richter et al. (Drug Metab Dispos 27:21-25, 1999). In contrast, alemtuzumab clears with a half-life of 15-21 days, and the blood concentration at a lympholytic level persists for up to 60 days in patients, resulting in the depletion of donor T cells after transplantation (Morris et al., 2003, Blood 102:404-406; Rebello et al., 2001, Cytotherapy 3:261-267). Thus, donor T cells are expected to be less influenced by IMMU-114 than by alemtuzumab, allowing the donor T cell-mediated third-party immunity to be maximally preserved.

Taken together, these studies demonstrate that IMMU-114 has the potential to be a novel component of the allograft conditioning regimen, with more efficiency, higher safety, and wider applicability, especially in patients with compromised immunity, compared to currently available agents.

### Example 3

### Effect of Anti-HLA-DR Antibody is Mediated Through ERK and JNK MAP Kinase Signaling Pathways

We examined the reactivity and cytotoxicity of the humanized anti-HLA-DR antibody hL243γ4P (IMMU-114) on a panel of leukemia cell lines. hL243γ4P bound to the cell surface of 2/3 AML, 2/2 mantle cell, 4/4 ALL, 1/1 hairy cell leukemia, and 2/2 CLL cell lines, but not on the 1 CML cell line tested (not shown). Cytotoxicity assays demonstrated that hL243γ4P was toxic to 2/2 mantle cell, 2/2 CLL, 3/4 ALL, and 1/1 hairy cell leukemia cell lines, but did not kill 3/3 AML cell lines despite positive staining (not shown). As expected, the CML cell line was also not killed by hL243γ4P (not shown).

The ex vivo effects of various antibodies on whole blood was examined. hL243γ4P resulted in significantly less B cell depletion than rituximab and veltuzumab (not shown), consistent with an earlier report (Nagy, et al, J Mol Med 2003;81:757-65) which suggested that anti-HLA-DR MAbs kill activated, but not resting normal B cells, in addition to tumor cells. This suggests a dual requirement for both MHC-II expression and cell activation for antibody-induced death, and implies that because the majority of peripheral B cells are resting, the potential side effect due to killing of normal B cells may be minimal. T-cells are unaffected.

The effects of ERK, JNK and ROS inhibitors on hL243γ4P mediated apoptosis in Raji cells was examined. hL243γ4P cytotoxicity correlates with activation of ERK and JNK signaling and differentiates the mechanism of action of hL243γ4P cytotoxicity from that of anti-CD20 MAbs (not shown). hL243γ4P also changes mitochondrial membrane potential and generates ROS in Raji cells (not shown). Inhibition of ERK, JNK, or ROS by specific inhibitors partially abrogates the apoptosis. Inhibition of 2 or more pathways abolishes the apoptosis.

Signaling pathways were studied to elucidate why cytotoxicity does not always correlate with antigen expression in the malignant B-cell lines examined. Various pathways were compared in IMMU-114—sensitive and —resistant HLA-DR—expressing cell lines. The AML lines, Kasumi-3 and GDM-1, were used as examples of HLA-DR⁺ cell lines resistant to IMMU-114 cytotoxicity. IMMU-114—sensitive cells included NHL (Raji), MCL (Jeko-1 and Granta-519), CLL (WAC and MEC-1), and ALL (REH and MN60). Results of Western blot analyses of these cell lines revealed that IMMU-114 induces phosphorylation and activation of ERK and JNK mitogen activated protein (MAP) kinases in all the cells defined as IMMU-114−sensitive by the cytotoxicity assays, but not the IMMU-114—resistant cell lines, Kasumi-3 and GDM-1 (data not shown). p38 MAP kinase was found to be constitutively active in these cell lines, and no further activation beyond basal levels was noted (data not shown).

Two methods were used to confirm the importance of the ERK and JNK signaling pathways in the IMMU-114 mechanism of action. These involved use of specific chemical inhibitors of these pathways and siRNA inhibition. ERK, JNK, and ROS inhibitors used were: NAC (5 mM) blocks ROS, U0126 (10 μM) blocks MEK phosphorylation and the ERK1/2 pathway, and SP600125 (10 μM) blocks the JNK pathway. Inhibition of ERK, JNK, or ROS by their respective inhibitors decreased apoptosis in Raji cells, although the inhibition was not complete when any single inhibitor was used (not shown). This may have been the result of activation of multiple pathways because inhibition of 2 or more pathways by specific inhibitors abolished the IMMU-114—induced apoptosis (not shown). Transfection of Raji cells with siERK and siJNK RNAs effectively lowered the expression of ERK and JNK proteins and significantly inhibited IMMU-114—induced apoptosis (not shown) validating the role of these pathways in IMMU-114 cell killing.

The AML lines, Kasumi-3 and GDM-1, were resistant to apoptosis mediated by IMMU-114 (as measured by annexin V, data not shown). Significant changes in mitochondrial membrane potential and generation of ROS also were not observed on treatment of these AML cell lines with IMMU-114 (not shown). Sensitive lines, such as Raji, showed a greater degree of homotypic aggregation on treatment with IMMU-114, whereas aggregation was not observed in AML lines, such as Kasumi-3 (data not shown).

Activation of ERK1/2 and JNK signaling pathways was also assessed in CLL patient samples (not shown). Patient cells were incubated with IMMU-114 for 4 hours because the cells in these samples were much smaller than those of the established cell lines. Moreover, the shorter incubation time avoids the risk of higher apoptosis and cell death. Similar to our observations in the IMMU-114—sensitive cell lines, activation and phosphorylation of the ERK1/2 and JNK pathways were observed in the CLL patient cells, indicating the generation of stress in these samples (not shown). Almost 4- to 5-fold activation of ERK and JNK pathways was observed on incubation with IMMU-114 over untreated controls, although no such activation was seen on treatment with rituximab or milatuzumab (not shown).

To further investigate the molecular mechanism whereby IMMU-114 induces cell death, we investigated the effect of IMMU-114 on changes in mitochondrial membrane potential and production of ROS. Treatment with IMMU-114 induced a time-dependent mitochondrial membrane depolarization that could be detected in Raji cells as well as in other sensitive lines (not shown). A time-course analysis in Raji cells indicated a change in mitochondrial membrane depolarization of 46% in as little as 30 minutes of treatment, and a further increase to 66% in 24 hours (not shown). Similar changes in ROS levels were observed (not shown). A thirty minute incubation with IMMU-114 induced a 24% change in ROS levels that increased to 33% to 44% on overnight incubation (not shown). Preincubation of Raji cells with the ROS inhibitor NAC blocked the generation of ROS on treatment with IMMU-114; only 8% ROS was observed in IMMU-114 plus NAC-treated cells (not shown). Changes in mitochondrial membrane potential were also abrogated by the ROS inhibitor (not shown). These observations suggest that ROS generation plays a crucial role in IMMU-114—induced cell death and are consistent with the action of IMMU-114 on ROS being an early effect occurring before apoptosis.

Discussion

To characterize the cytotoxic mechanism of IMMU-114, we compared the activation of ERK, JNK, and p38 MAP kinases in our panel of cell lines and CLL patient cells. We found that JNK1/2 and ERK1/2 phosphorylation was up-regulated after exposure of cells to IMMU-114 in sensitive cell lines, such as the CLL patient cells, and the Raji and Jeko-1 cell lines, but not in the IMMU-114—resistant AML cell lines, such as Kasumi-3 and GDM-1. We observed up to 5-fold activation of the ERK and JNK signaling pathways on treatment with IMMU-114 at a modest 10-nM concentration. p38 MAP kinase was found to be constitutively active in these cell lines, and no further activation beyond basal levels was noted. Inhibition of the ERK and JNK signaling cascades by their respective inhibitors could modestly inhibit the apoptosis induced by IMMU-114. However, apoptosis was completely inhibited when 2 inhibitors were used together, indicating the activation of multiple MAP kinases by IMMU-114. IMMU-114—induced apoptosis was also significantly inhibited by siERK and siJNK RNAs. Thus, IMMU-114 cytotoxicity correlates with activation of ERK and JNK signaling. In addition, the results of these studies differentiate the mechanism of action of IMMU-114 cytotoxicity from that of the anti-CD74 (milatuzumab) and anti- CD20 MAbs.

### Example 4

### Preparation of Dock-and-Lock (DNL) Constructs DDD and AD Fusion Proteins

The DNL technique can be used to make dimers, trimers, tetramers, hexamers, etc. comprising virtually any antibody, antibody fragment, cytokine or other effector moiety. For certain preferred embodiments, antibodies, cytokines, toxins or other protein or peptide effectors may be produced as fusion proteins comprising either a dimerization and docking domain (DDD) or anchoring domain (AD) sequence. Although in preferred embodiments the DDD and AD moieties may be joined to antibodies, antibody fragments, cytokines or other effectors as fusion proteins, the skilled artisan will realize that other methods of conjugation exist, such as chemical cross-linking, click chemistry reaction, etc.

The technique is not limiting and any protein or peptide of use may be produced as an AD or DDD fusion protein for incorporation into a DNL construct. Where chemical cross-linking is utilized, the AD and DDD conjugates may comprise any molecule that may be cross-linked to an AD or DDD sequence using any cross-linking technique known in the art. In certain exemplary embodiments, a dendrimer or other polymeric moiety such as polyethyleneimine or polyethylene glycol (PEG), may be incorporated into a DNL construct, as described in further detail below.

For different types of DNL constructs, different AD or DDD sequences may be utilized. Exemplary DDD and AD sequences are provided below.

DDD1:
(SEQ ID NO: 45)
SHIQIPPGLTELLQGYTVEVLRQQPPDLVEFAVEYFTRLREARA
DDD2:
(SEQ ID NO: 46)
CGHIQIPPGLTELLQGYTVEVLRQQPPDLVEFAVEYFTRLREARA
AD1:
(SEQ ID NO: 47)
QIEYLAKQIVDNAIQQA
AD2:
(SEQ ID NO: 48)
CGQIEYLAKQIVDNAIQQAGC

The skilled artisan will realize that DDD1 and DDD2 comprise the DDD sequence of the human RIIα form of protein kinase A. However, in alternative embodiments, the DDD and AD moieties may be based on the DDD sequence of the human RIα form of protein kinase A and a corresponding AKAP sequence, as exemplified in DDD3, DDD3C and AD3 below.

DDD3
(SEQ ID NO: 49)
SLRECELYVQKHNIQALLKDSIVQLCTARPERPMAFLREYFERLEKEEA
K
DDD3C
(SEQ ID NO: 50)
MSCGGSLRECELYVQKHNIQALLKDSIVQLCTARPERPMAFLREYFERL
EKEEAK
AD3
(SEQ ID NO: 51)
CGFEELAWKIAKMIWSDVFQQGC

Expression Vectors

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (VH and VL) sequences. Using molecular biology tools known to those skilled in the art, these IgG expression vectors can be converted into Fab-DDD or Fab-AD expression vectors. To generate Fab-DDD expression vectors, the coding sequences for the hinge, CH2 and CH3 domains of the heavy chain are replaced with a sequence encoding the first 4 residues of the hinge, a 14 residue Gly-Ser linker and the first 44 residues of human RIIα (referred to as DDD1). To generate Fab-AD expression vectors, the sequences for the hinge, CH2 and CH3 domains of IgG are replaced with a sequence encoding the first 4 residues of the hinge, a 15 residue Gly-Ser linker and a 17 residue synthetic AD called AKAP-IS (referred to as AD1), which was generated using bioinformatics and peptide array technology and shown to bind RIIα dimers with a very high affinity (0.4 nM). See Alto, et al. Proc. Natl. Acad. Sci., U.S.A (2003), 100:4445-50.

Two shuttle vectors were designed to facilitate the conversion of IgG-pdHL2 vectors to either Fab-DDD1 or Fab-AD1 expression vectors, as described below.

Preparation of CH1

The CH1 domain was amplified by PCR using the pdHL2 plasmid vector as a template. The left PCR primer consisted of the upstream (5′) end of the CH1 domain and a SacII restriction endonuclease site, which is 5′ of the CH1 coding sequence. The right primer consisted of the sequence coding for the first 4 residues of the hinge (PKSC, SEQ ID NO:98) followed by four glycines and a serine, with the final two codons (GS) comprising a Barn HI restriction site. The 410 by PCR amplimer was cloned into the PGEMT® PCR cloning vector (PROMEGA®, Inc.) and clones were screened for inserts in the T7 (5′) orientation.

A duplex oligonucleotide was synthesized to code for the amino acid sequence of DDD1 preceded by 11 residues of the linker peptide, with the first two codons comprising a BamHI restriction site. A stop codon and an EagI restriction site are appended to the 3′end. The encoded polypeptide sequence is shown below.

(SEQ ID NO: 52)
GSGGGGSGGGGSHIQIPPGLTELLQGYTVEVLRQQPPDLVEFAVEYFTR
LREARA

Two oligonucleotides, designated RIIA1-44 top and RIIA1-44 bottom, which overlap by 30 base pairs on their 3′ ends, were synthesized and combined to comprise the central 154 base pairs of the 174 by DDD1 sequence. The oligonucleotides were annealed and subjected to a primer extension reaction with Taq polymerase. Following primer extension, the duplex was amplified by PCR. The amplimer was cloned into PGEMT® and screened for inserts in the T7 (5′) orientation.

A duplex oligonucleotide was synthesized to code for the amino acid sequence of AD1 preceded by 11 residues of the linker peptide with the first two codons comprising a BamHI restriction site. A stop codon and an EagI restriction site are appended to the 3′end. The encoded polypeptide sequence is shown below.

(SEQ ID NO: 53)
GSGGGGSGGGGSQIEYLAKQIVDNAIQQA

Two complimentary overlapping oligonucleotides encoding the above peptide sequence, designated AKAP-IS Top and AKAP-IS Bottom, were synthesized and annealed. The duplex was amplified by PCR. The amplimer was cloned into the PGEMT® vector and screened for inserts in the T7 (5′) orientation.

Ligating DDD1 with CH1

A 190 by fragment encoding the DDD1 sequence was excised from PGEMT® with BamHI and NotI restriction enzymes and then ligated into the same sites in CH1-PGEMT® to generate the shuttle vector CH1-DDD1-PGEMT®.

Ligating AD1 with CH1

A 110 by fragment containing the AD1 sequence was excised from PGEMT® with BamHI and NotI and then ligated into the same sites in CH1-PGEMT® to generate the shuttle vector CH1-AD1-PGEMT®.

Cloning CH1-DDD1 or CH1-AD1 into pdHL2-based vectors

With this modular design either CH1-DDD1 or CH1-AD1 can be incorporated into any IgG construct in the pdHL2 vector. The entire heavy chain constant domain is replaced with one of the above constructs by removing the SacII/EagI restriction fragment (CH1-CH3) from pdHL2 and replacing it with the SacII/EagI fragment of CH1-DDD1 or CH1-AD1, which is excised from the respective pGemT shuttle vector.

Construction of h679-Fd-AD1-pdHL2

h679-Fd-AD1-pdHL2 is an expression vector for production of h679 Fab with AD1 coupled to the carboxyl terminal end of the CH1 domain of the Fd via a flexible Gly/Ser peptide spacer composed of 14 amino acid residues. A pdHL2-based vector containing the variable domains of h679 was converted to h679-Fd-AD1-pdHL2 by replacement of the SacII/EagI fragment with the CHI-AD1 fragment, which was excised from the CH1-AD1-SV3 shuttle vector with SacII and EagI.

Construction of C-DDD1-Fd-hMN-14-pdHL2

C-DDD1-Fd-hMN-14-pdHL2 is an expression vector for production of a stable dimer that comprises two copies of a fusion protein C-DDD1-Fab-hMN-14, in which DDD1 is linked to hMN-14 Fab at the carboxyl terminus of CH1 via a flexible peptide spacer. The plasmid vector hMN-14(I)-pdHL2, which has been used to produce hMN-14 IgG, was converted to C-DDD1-Fd-hMN-14-pdHL2 by digestion with SacII and EagI restriction endonucleases to remove the CH1-CH3 domains and insertion of the CH1-DDD1 fragment, which was excised from the CH1-DDD1-SV3 shuttle vector with SacII and EagI.

The same technique has been utilized to produce plasmids for Fab expression of a wide variety of known antibodies, such as hLL1, hLL2, hPAM4, hR1, hRS7, hMN-14, hMN-15, hA19, hA20 and many others. Generally, the antibody variable region coding sequences were present in a pdHL2 expression vector and the expression vector was converted for production of an AD- or DDD-fusion protein as described above. The AD- and DDD-fusion proteins comprising a Fab fragment of any of such antibodies may be combined, in an approximate ratio of two DDD-fusion proteins per one AD-fusion protein, to generate a trimeric DNL construct comprising two Fab fragments of a first antibody and one Fab fragment of a second antibody.

Construction of N-DDD1-Fd-hMN-14-pdHL2

N-DDD1-Fd-hMN-14-pdHL2 is an expression vector for production of a stable dimer that comprises two copies of a fusion protein N-DDD1-Fab-hMN-14, in which DDD1 is linked to hMN-14 Fab at the amino terminus of VH via a flexible peptide spacer. The expression vector was engineered as follows. The DDD1 domain was amplified by PCR.

As a result of the PCR, an NcoI restriction site and the coding sequence for part of the linker containing a BamHI restriction were appended to the 5′ and 3′ ends, respectively. The 170 by PCR amplimer was cloned into the pGemT vector and clones were screened for inserts in the T7 (5′) orientation. The 194 by insert was excised from the pGemT vector with NcoI and Sail restriction enzymes and cloned into the SV3 shuttle vector, which was prepared by digestion with those same enzymes, to generate the intermediate vector DDD1-SV3.

The hMN-14 Fd sequence was amplified by PCR. As a result of the PCR, a BamHI restriction site and the coding sequence for part of the linker were appended to the 5′ end of the amplimer. A stop codon and EagI restriction site was appended to the 3′ end. The 1043 by amplimer was cloned into pGemT. The hMN-14-Fd insert was excised from pGemT with BamHI and EagI restriction enzymes and then ligated with DDD1-SV3 vector, which was prepared by digestion with those same enzymes, to generate the construct N-DDD1-hMN-14Fd-SV3.

The N-DDD1-hMN-14 Fd sequence was excised with XhoI and EagI restriction enzymes and the 1.28 kb insert fragment was ligated with a vector fragment that was prepared by digestion of C-hMN-14-pdHL2 with those same enzymes. The final expression vector was N-DDD1-Fd-hMN-14-pDHL2. The N-linked Fab fragment exhibited similar DNL complex formation and antigen binding characteristics as the C-linked Fab fragment (not shown).

C-DDD2-Fd-hMN-14-pdHL2

C-DDD2-Fd-hMN-14-pdHL2 is an expression vector for production of C-DDD2-Fab-hMN-14, which possesses a dimerization and docking domain sequence of DDD2 appended to the carboxyl terminus of the Fd of hMN-14 via a 14 amino acid residue Gly/Ser peptide linker. The fusion protein secreted is composed of two identical copies of hMN-14 Fab held together by non-covalent interaction of the DDD2 domains.

The expression vector was engineered as follows. Two overlapping, complimentary oligonucleotides, which comprise the coding sequence for part of the linker peptide and residues 1-13 of DDD2, were made synthetically. The oligonucleotides were annealed and phosphorylated with T4 PNK, resulting in overhangs on the 5′ and 3′ ends that are compatible for ligation with DNA digested with the restriction endonucleases BamHI and PstI, respectively.

The duplex DNA was ligated with the shuttle vector CH1-DDD1-PGEMT®, which was prepared by digestion with BamHI and PstI, to generate the shuttle vector CH1-DDD2-PGEMT®. A 507 by fragment was excised from CH1-DDD2-PGEMT® with SacII and EagI and ligated with the IgG expression vector hMN-14(I)-pdHL2, which was prepared by digestion with SacII and EagI. The final expression construct was designated C-DDD2-Fd-hMN-14-pdHL2. Similar techniques have been utilized to generated DDD2-fusion proteins of the Fab fragments of a number of different humanized antibodies.

h679-Fd-AD2-pdHL2

h679-Fab-AD2, was designed to pair as B to C-DDD2-Fab-hMN-14 as A. h679-Fd-AD2-pdHL2 is an expression vector for the production of h679-Fab-AD2, which possesses an anchoring domain sequence of AD2 appended to the carboxyl terminal end of the CH1 domain via a 14 amino acid residue Gly/Ser peptide linker. AD2 has one cysteine residue preceding and another one following the anchor domain sequence of AD1.

The expression vector was engineered as follows. Two overlapping, complimentary oligonucleotides (AD2 Top and AD2 Bottom), which comprise the coding sequence for AD2 and part of the linker sequence, were made synthetically. The oligonucleotides were annealed and phosphorylated with T4 PNK, resulting in overhangs on the 5′ and 3′ ends that are compatible for ligation with DNA digested with the restriction endonucleases BamHI and SpeI, respectively.

The duplex DNA was ligated into the shuttle vector CH1-AD1-PGEMT®, which was prepared by digestion with BamHI and SpeI, to generate the shuttle vector CH1-AD2-PGEMT®. A 429 base pair fragment containing CH1 and AD2 coding sequences was excised from the shuttle vector with SacII and EagI restriction enzymes and ligated into h679-pdHL2 vector that prepared by digestion with those same enzymes. The final expression vector is h679-Fd-AD2-pdHL2.

### Example 5

### Generation of TF1 DNL Construct

A large scale preparation of a DNL construct, referred to as TF1, was carried out as follows. N-DDD2-Fab-hMN-14 (Protein L-purified) and h679-Fab-AD2 (IMP-291-purified) were first mixed in roughly stoichiometric concentrations in 1 mM EDTA, PBS, pH 7.4. Before the addition of TCEP, SE-HPLC did not show any evidence of a₂b formation (not shown). Instead there were peaks representing a₄ (7.97 min; 200 kDa), a₂ (8.91 min; 100 kDa) and B (10.01 min; 50 kDa). Addition of 5 mM TCEP rapidly resulted in the formation of the a₂b complex as demonstrated by a new peak at 8.43 min, consistent with a 150 kDa protein (not shown). Apparently there was excess B in this experiment as a peak attributed to h679-Fab-AD2 (9.72 min) was still evident yet no apparent peak corresponding to either a₂ or a₄ was observed. After reduction for one hour, the TCEP was removed by overnight dialysis against several changes of PBS. The resulting solution was brought to 10% DMSO and held overnight at room temperature.

When analyzed by SE-HPLC, the peak representing a₂b appeared to be sharper with a slight reduction of the retention time by 0.1 min to 8.31 min (not shown), which, based on our previous findings, indicates an increase in binding affinity. The complex was further purified by IMP-29l affinity chromatography to remove the kappa chain contaminants. As expected, the excess h679-AD2 was co-purified and later removed by preparative SE-HPLC (not shown).

TF1 is a highly stable complex. When TF1 was tested for binding to an HSG (IMP-239) sensorchip, there was no apparent decrease of the observed response at the end of sample injection. In contrast, when a solution containing an equimolar mixture of both C-DDD1-Fab-hMN-14 and h679-Fab-AD1 was tested under similar conditions, the observed increase in response units was accompanied by a detectable drop during and immediately after sample injection, indicating that the initially formed a₂b structure was unstable. Moreover, whereas subsequent injection of WI2 gave a substantial increase in response units for TF1, no increase was evident for the C-DDD1/AD1 mixture.

The additional increase of response units resulting from the binding of WI2 to TF1 immobilized on the sensorchip corresponds to two fully functional binding sites, each contributed by one subunit of N-DDD2-Fab-hMN-14. This was confirmed by the ability of TF1 to bind two Fab fragments of WI2 (not shown). When a mixture containing h679-AD2 and N-DDD1-hMN14, which had been reduced and oxidized exactly as TF1, was analyzed by BIAcore, there was little additional binding of WI2 (not shown), indicating that a disulfide-stabilized a₂b complex such as TF1 could only form through the interaction of DDD2 and AD2.

Two improvements to the process were implemented to reduce the time and efficiency of the process. First, a slight molar excess of N-DDD2-Fab-hMN-14 present as a mixture of a₄/a₂ structures was used to react with h679-Fab-AD2 so that no free h679-Fab-AD2 remained and any a₄/a₂ structures not tethered to h679-Fab-AD2, as well as light chains, would be removed by IMP-291 affinity chromatography. Second, hydrophobic interaction chromatography (HIC) has replaced dialysis or diafiltration as a means to remove TCEP following reduction, which would not only shorten the process time but also add a potential viral removing step. N-DDD2-Fab-hMN-14 and 679-Fab-AD2 were mixed and reduced with 5 mM TCEP for 1 hour at room temperature. The solution was brought to 0.75 M ammonium sulfate and then loaded onto a Butyl FF HIC column. The column was washed with 0.75 M ammonium sulfate, 5 mM EDTA, PBS to remove TCEP. The reduced proteins were eluted from the HIC column with PBS and brought to 10% DMSO. Following incubation at room temperature overnight, highly purified TF1 was isolated by IMP-291 affinity chromatography (not shown). No additional purification steps, such as gel filtration, were required.

### Example 6

### Generation of TF2 DNL Construct

A trimeric DNL construct designated TF2 was obtained by reacting C-DDD2-Fab-hMN-14 with h679-Fab-AD2. A pilot batch of TF2 was generated with >90% yield as follows. Protein L-purified C-DDD2-Fab-hMN-14 (200 mg) was mixed with h679-Fab-AD2 (60 mg) at a 1.4:1 molar ratio. The total protein concentration was 1.5 mg/ml in PBS containing 1 mM EDTA. Subsequent steps involved TCEP reduction, HIC chromatography, DMSO oxidation, and IMP 291 affinity chromatography. Before the addition of TCEP, SE-HPLC did not show any evidence of a₂b formation. Addition of 5 mM TCEP rapidly resulted in the formation of a₂b complex consistent with a 157 kDa protein expected for the binary structure. TF2 was purified to near homogeneity by IMP 291 affinity chromatography (not shown). IMP 291 is a synthetic peptide containing the HSG hapten to which the 679 Fab binds (Rossi et al., 2005, Clin Cancer Res 11:7122s-29s). SE-HPLC analysis of the IMP 291 unbound fraction demonstrated the removal of a₄, a₂ and free kappa chains from the product (not shown).

The functionality of TF2 was determined by BIACORE® assay. TF2, C-DDD1-hMN-14+h679-AD1 (used as a control sample of noncovalent a₁b complex), or C-DDD2-hMN-14+h679-AD2 (used as a control sample of unreduced a₂ and b components) were diluted to 1 μg/ml (total protein) and passed over a sensorchip immobilized with HSG. The response for TF2 was approximately two-fold that of the two control samples, indicating that only the h679-Fab-AD component in the control samples would bind to and remain on the sensorchip. Subsequent injections of WI2 IgG, an anti-idiotype antibody for hMN-14, demonstrated that only TF2 had a DDD-Fab-hMN-14 component that was tightly associated with h679-Fab-AD as indicated by an additional signal response. The additional increase of response units resulting from the binding of WI2 to TF2 immobilized on the sensorchip corresponded to two fully functional binding sites, each contributed by one subunit of C-DDD2-Fab-hMN-14. This was confirmed by the ability of TF2 to bind two Fab fragments of WI2 (not shown).

### Example 7

### Production of AD- and DDD-Linked Fab and IgG Fusion Proteins From Multiple Antibodies

Using the techniques described in the preceding Examples, the IgG and Fab fusion proteins shown in Table 2 were constructed and incorporated into DNL constructs. The fusion proteins retained the antigen-binding characteristics of the parent antibodies and the DNL constructs exhibited the antigen-binding activities of the incorporated antibodies or antibody fragments.

TABLE 2
Fusion proteins comprising IgG or Fab

| Fusion Protein | Binding Specificity |
|---|---|
| C-AD1-Fab-h679 | HSG |
| C-AD2-Fab-h679 | HSG |
| C-(AD)₂-Fab-h679 | HSG |
| C-AD2-Fab-h734 | Indium-DTPA |
| C-AD2-Fab-hA20 | CD20 |
| C-AD2-Fab-hA20L | CD20 |
| C-AD2-Fab-hL243 | HLA-DR |
| C-AD2-Fab-hLL2 | CD22 |
| N-AD2-Fab-hLL2 | CD22 |
| C-AD2-IgG-hMN-14 | CEACAM5 |
| C-AD2-IgG-hR1 | IGF-1R |
| C-AD2-IgG-hRS7 | EGP-1 |
| C-AD2-IgG-hPAM4 | MUC |
| C-AD2-IgG-hLL1 | CD74 |
| C-DDD1-Fab-hMN-14 | CEACAM5 |
| C-DDD2-Fab-hMN-14 | CEACAM5 |
| C-DDD2-Fab-h679 | HSG |
| C-DDD2-Fab-hA19 | CD19 |
| C-DDD2-Fab-hA20 | CD20 |
| C-DDD2-Fab-hAFP | AFP |
| C-DDD2-Fab-hL243 | HLA-DR |
| C-DDD2-Fab-hLL1 | CD74 |
| C-DDD2-Fab-hLL2 | CD22 |
| C-DDD2-Fab-hMN-3 | CEACAM6 |
| C-DDD2-Fab-hMN-15 | CEACAM6 |
| C-DDD2-Fab-hPAM4 | MUC |
| C-DDD2-Fab-hR1 | IGF-1R |
| C-DDD2-Fab-hRS7 | EGP-1 |
| N-DDD2-Fab-hMN-14 | CEACAM5 |

### Example 8

### Sequence Variants for DNL

In addition to the sequences of DDD1, DDD2, DDD3, DDD3C, AD1, AD2 and AD3 described above, other sequence variants of AD and/or DDD moieties may be utilized in construction of the DNL complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 54)
SLRECELYVQKHNIQALLKDVSIVQLCTARPERPMAFLREYFEKLEKEE
AK
PKA RIβ
(SEQ ID NO: 55)
SLKGCELYVQLHGIQQVLKDCIVHLCISKPERPMKFLREHFEKLEKEEN
RQILA
PKA RIIα
(SEQ ID NO: 56)
SHIQIPPGLTELLQGYTVEVGQQPPDLVDFAVEYFTRLREARRQ
PKA RIIβ
(SEQ ID NO: 57)
SIEIPAGLTELLQGFTVEVLRHQPADLLEFALQHFTRLQQENER

The structure-function relationships of the AD and DDD domains have been the subject of investigation. (See, e.g., Burns-Hamuro et al., 2005, Protein Sci 14:2982-92; Carr et al., 2001, J Biol Chem 276:17332-38; Alto et al., 2003, Proc Natl Acad Sci USA 100:4445-50; Hundsrucker et al., 2006, Biochem J 396:297-306; Stokka et al., 2006, Biochem J 400:493-99; Gold et al., 2006, Mol Cell 24:383-95; Kinderman et al., 2006, Mol Cell 24:397-408, the entire text of each of which is incorporated herein by reference.)

For example, Kinderman et al. (2006, Mol Cell 24:397-408) examined the crystal structure of the AD-DDD binding interaction and concluded that the human DDD sequence contained a number of conserved amino acid residues that were important in either dimer formation or AKAP binding, underlined in SEQ ID NO:45 below. (See FIG. 1 of Kinderman et al., 2006, incorporated herein by reference.) The skilled artisan will realize that in designing sequence variants of the DDD sequence, one would desirably avoid changing any of the underlined residues, while conservative amino acid substitutions might be made for residues that are less critical for dimerization and AKAP binding.

(SEQ ID NO: 45)
SHIQIPPGLTELLQGYTVEVLRQQPPDLVEFAVEYFTRLREARA

As discussed in more detail below, conservative amino acid substitutions have been characterized for each of the twenty common L-amino acids. Thus, based on the data of Kinderman (2006) and conservative amino acid substitutions, potential alternative DDD sequences based on SEQ ID NO:45 are shown in Table 3. In devising Table 3, only highly conservative amino acid substitutions were considered. For example, charged residues were only substituted for residues of the same charge, residues with small side chains were substituted with residues of similar size, hydroxyl side chains were only substituted with other hydroxyls, etc. Because of the unique effect of proline on amino acid secondary structure, no other residues were substituted for proline. The skilled artisan will realize that a very large number of alternative species within the genus of DDD moieties can be constructed by standard techniques, for example using a commercial peptide synthesizer or well known site-directed mutagenesis techniques. The effect of the amino acid substitutions on AD moiety binding may also be readily determined by standard binding assays, for example as disclosed in Alto et al. (2003, Proc Natl Acad Sci USA 100:4445-50).

TABLE 3
Conservative Amino Acid Substitutions in DDD1 (SEQ ID NO: 45).
Consensus sequence disclosed as SEQ ID NO: 58.

| S | H | I | Q | I | P | P | G | L | T | E | L | L | Q | G | Y | T | V | E | V | L | R |
|---|---|---|---|---|---|---|---|---|---|---|---|---|---|---|---|---|---|---|---|---|---|
| T | K | | N | | | | A | | S | D | | | N | A | | S | | D | | | K |
| | R | | | | | | | | | | | | | | | | | | | | |
| Q | Q | P | P | D | L | V | E | F | A | V | E | Y | F | T | R | L | R | E | A | R | A |
| N | N | | | E | | | D | | L | | D | | | S | K | | K | D | L | K | L |
| | | | | | | | | | I | | | | | | | | | | I | | I |
| | | | | | | | | | V | | | | | | | | | | V | | V |

Alto et al. (2003, Proc Natl Acad Sci USA 100:4445-50) performed a bioinformatic analysis of the AD sequence of various AKAP proteins to design an RII selective AD sequence called AKAP-IS (SEQ ID NO:47), with a binding constant for DDD of 0.4 nM. The AKAP-IS sequence was designed as a peptide antagonist of AKAP binding to PKA. Residues in the AKAP-IS sequence where substitutions tended to decrease binding to DDD are underlined in SEQ ID NO:47 below. The skilled artisan will realize that in designing sequence variants of the AD sequence, one would desirably avoid changing any of the underlined residues, while conservative amino acid substitutions might be made for residues that are less critical for DDD binding. Table 4 shows potential conservative amino acid substitutions in the sequence of AKAP-IS (AD1, SEQ ID NO:47), similar to that shown for DDD1 (SEQ ID NO:45) in Table 3 above.

A large number of AD moiety sequences could be made, tested and used by the skilled artisan, based on the data of Alto et al. (2003). It is noted that FIG. 2 of Alto (2003) shows an even large number of potential amino acid substitutions that may be made, while retaining binding activity to DDD moieties, based on actual binding experiments.

AKAP-IS
(SEQ ID NO: 47)
QIEYLAKQIVDNAIQQA

TABLE 4
Conservative Amino Acid Substitutions in AD1 (SEQ ID NO: 47).
Consensus sequence disclosed as SEQ ID NO: 59.

| Q | I | E | Y | L | A | K | Q | I | V | D | N | A | I | Q | Q | A |
|---|---|---|---|---|---|---|---|---|---|---|---|---|---|---|---|---|
| N | L | D | F | I | | R | N | | | E | Q | | | N | N | L |
| | V | | T | V | | | | | | | | | | | | I |
| | | | S | | | | | | | | | | | | | V |

Gold et al. (2006, Mol Cell 24:383-95) utilized crystallography and peptide screening to develop a SuperAKAP-IS sequence (SEQ ID NO:60), exhibiting a five order of magnitude higher selectivity for the RII isoform of PKA compared with the RI isoform. Underlined residues indicate the positions of amino acid substitutions, relative to the AKAP-IS sequence, which increased binding to the DDD moiety of RIIα. In this sequence, the N-terminal Q residue is numbered as residue number 4 and the C-terminal A residue is residue number 20. Residues where substitutions could be made to affect the affinity for RIIα were residues 8, 11, 15, 16, 18, 19 and 20 (Gold et al., 2006). It is contemplated that in certain alternative embodiments, the SuperAKAP-IS sequence may be substituted for the AKAP-IS AD moiety sequence to prepare DNL constructs. Other alternative sequences that might be substituted for the AKAP-IS AD sequence are shown in SEQ ID NO:61-63. Substitutions relative to the AKAP-IS sequence are underlined. It is anticipated that, as with the AD2 sequence shown in SEQ ID NO:48, the AD moiety may also include the additional N-terminal residues cysteine and glycine and C-terminal residues glycine and cysteine.

SuperAKAP-IS
(SEQ ID NO: 60)
QIEYVAKQIVDYAIHQA
Alternative AKAP sequences
(SEQ ID NO: 61)
QIFYKAKQIVDHAIHQA
(SEQ ID NO: 62)
QIEYHAKQIVDHAIHQA
(SEQ ID NO: 63)
QIEYVAKQIVDHAIHQA

FIG. 2 of Gold et al. disclosed additional DDD-binding sequences from a variety of AKAP proteins, shown below.

RII-Specific AKAPs
AKAP-KL
(SEQ ID NO: 64)
PLEYQAGLLVQNAIQQAI
AKAP79
(SEQ ID NO: 65)
LLIETASSLVKNAIQLSI
AKAP-Lbc
(SEQ ID NO: 66)
LIEEAASRIVDAVIEQVK
RI-Specific AKAPs
AKAPce
(SEQ ID NO: 67)
ALYQFADRFSELVISEAL
RIAD
(SEQ ID NO: 68)
LEQVANQLADQIIKEAT
PV38
(SEQ ID NO: 69)
FEELAWKIAKMIWSDVF
Dual-Specificity AKAPs
AKAP7
(SEQ ID NO: 70)
ELVRLSKRLVENAVLKAV
MAP2D
(SEQ ID NO: 71)
TAEEVSARIVQVVTAEAV
DAKAP1
(SEQ ID NO: 72)
QIKQAAFQLISQVILEAT
DAKAP2
(SEQ ID NO: 73)
LAWKIAKMIVSDVMQQ

Stokka et al. (2006, Biochem J 400:493-99) also developed peptide competitors of AKAP binding to PKA, shown in SEQ ID NO:74-76. The peptide antagonists were designated as Ht31 (SEQ ID NO:74), RIAD (SEQ ID NO:75) and PV-38 (SEQ ID NO:76). The Ht-31 peptide exhibited a greater affinity for the RII isoform of PKA, while the RIAD and PV-38 showed higher affinity for RI.

Ht31
(SEQ ID NO: 74)
DLIEEAASRIVDAVIEQVKAAGAY
RIAD
(SEQ ID NO: 75)
LEQYANQLADQIIKEATE
PV-38
(SEQ ID NO: 76)
FEELAWKIAKMIWSDVFQQC

Hundsrucker et al. (2006, Biochem 3 396:297-306) developed still other peptide competitors for AKAP binding to PKA, with a binding constant as low as 0.4 nM to the DDD of the RII form of PKA. The sequences of various AKAP antagonistic peptides are provided in Table 1 of Hundsrucker et al., reproduced in Table 5 below. AKAPIS represents a synthetic RII subunit-binding peptide. All other peptides are derived from the RII-binding domains of the indicated AKAPs.

TABLE 5
AKAP Peptide sequences

| | Peptide Sequence |
|---|---|
| AKAPIS | QIEYLAKQIVDNAIQQA |
| | (SEQ ID NO: 47) |
| AKAPIS-P | QIEYLAKQIPDNAIQQA |
| | (SEQ ID NO: 77) |
| Ht31 | KGADLIEEAASRIVDAVIEQVKAAG |
| | (SEQ ID NO: 78) |
| Ht31-P | KGADLIFEAASRIPDAPIEQVKAAG |
| | (SEQ ID NO: 79) |
| AKAP7δ-wt-pep | PEDAELVRLSKRLVENAVLKAVQQY |
| | (SEQ ID NO: 80) |
| AKAP7δ-L304T-pep | PEDAELVRTSKRLVENAVLKAVQQY |
| | (SEQ ID NO: 81) |
| AKAP7δ-L308D-pep | PEDAELVRLSKRDVENAVLKAVQQY |
| | (SEQ ID NO: 82) |
| AKAP7δ-P-pep | PEDAELVRLSKRLPENAVLKAVQQY |
| | (SEQ ID NO: 83) |
| AKAP7δ-PP-pep | PEDAELVRLSKRLPENAPLKAVQQY |
| | (SEQ ID NO: 84) |
| AKAP7δ-L314E-pep | PEDAELVRLSKRLVENAVEKAVQQY |
| | (SEQ ID NO: 85) |
| AKAP1-pep | EEGLDRNEEIKRAAFQIISQVISEA |
| | (SEQ ID NO: 86) |
| AKAP2-pep | LVDDPLEYQAGLLVQNAIQQAIAEQ |
| | (SEQ ID NO: 87) |
| AKAP5-pep | QYETLLIETASSLVKNAIQLSIEQL |
| | (SEQ ID NO: 88) |
| AKAP9-pep | LEKQYQEQLEEEVAKVIVSMSIAFA |
| | (SEQ ID NO: 89) |
| AKAP10-pep | NTDEAQEELAWKIAKMIVSDIMQQA |
| | (SEQ ID NO: 90) |
| AKAP11-pep | VNLDKKAVLAEKIVAEMEKAEREL |
| | (SEQ ID NO: 91) |
| AKAP12-pep | NGILELETKSSKLVQNIIQTAVDQF |
| | (SEQ ID NO: 92) |
| AKAP14-pep | TQDKNYEDELTQVALALVEDVINYA |
| | (SEQ ID NO: 93) |
| Rab32-pep | ETSAKDNINIEEAARFLVEKILVNH |
| | (SEQ ID NO: 94) |

Residues that were highly conserved among the AD domains of different AKAP proteins are indicated below by underlining with reference to the AKAP IS sequence (SEQ ID NO:47). The residues are the same as observed by Alto et al. (2003), with the addition of the C-terminal alanine residue. (See FIG. 4 of Hundsrucker et al. (2006), incorporated herein by reference.) The sequences of peptide antagonists with particularly high affinities for the RII DDD sequence were those of AKAP-IS, AKAP7δ-wt-pep, AKAP7δ-L304T-pep and AKAP7δ-L308D-pep.

AKAP-IS
(SEQ ID NO: 47)
QIEYLAKQIVDNAIQQA

Carr et al. (2001, J Biol Chem 276:17332-38) examined the degree of sequence homology between different AKAP-binding DDD sequences from human and non-human proteins and identified residues in the DDD sequences that appeared to be the most highly conserved among different DDD moieties. These are indicated below by underlining with reference to the human PKA RIIα DDD sequence of SEQ ID NO:45. Residues that were particularly conserved are further indicated by italics. The residues overlap with, but are not identical to those suggested by Kinderman et al. (2006) to be important for binding to AKAP proteins. The skilled artisan will realize that in designing sequence variants of DDD, it would be most preferred to avoid changing the most conserved residues (italicized), and it would be preferred to also avoid changing the conserved residues (underlined), while conservative amino acid substitutions may be considered for residues that are neither underlined nor italicized.

(SEQ ID NO: 45)
SHIQIPPGLTELLQGYTVEVLRQQPPDLVEFAVEYFTRLREARA

A modified set of conservative amino acid substitutions for the DDD1 (SEQ ID NO:45) sequence, based on the data of Carr et al. (2001) is shown in Table 6. The skilled artisan could readily derive alternative DDD amino acid sequences as disclosed above for Table 3 and Table 4.

TABLE 6
Conservative Amino Acid Substitutions in DDD1 (SEQ ID NO: 45).
Consensus sequence disclosed as SEQ ID NO: 95.

| S | H | I | Q | [symbol] | [symbol] | P | [symbol] | [symbol] | T | E | [symbol] | [symbol] | Q | [symbol] | [symbol] | [symbol] | V | [symbol] | [symbol] | [symbol] | [symbol] |
|---|---|---|---|---|---|---|---|---|---|---|---|---|---|---|---|---|---|---|---|---|---|
| T | | | N | | | | | | S | | | | | | | | I | | | | |
| | | | | | | | | | | | | | | | | | L | | | | |
| | | | | | | | | | | | | | | | | | A | | | | |
| Q | [symbol] | [symbol] | P | [symbol] | [symbol] | V | E | [symbol] | [symbol] | V | E | [symbol] | [symbol] | T | R | [symbol] | R | E | A | [symbol] | A |
| N | | | | | | | | | | I | D | | | S | K | | K | | L | | L |
| | | | | | | | | | | L | | | | | | | | | I | | I |
| | | | | | | | | | | A | | | | | | | | | V | | V |

The skilled artisan will realize that these and other amino acid substitutions in the DDD or AD amino acid sequences may be utilized to produce alternative species within the genus of AD or DDD moieties, using techniques that are standard in the field and only routine experimentation.

### Example 9

### Antibody-Dendrimer DNL Complex for siRNA

Cationic polymers, such as polylysine, polyethylenimine, or polyamidoamine (PAMAM)-based dendrimers, form complexes with nucleic acids. However, their potential applications as non-viral vectors for delivering therapeutic genes or siRNAs remain a challenge. One approach to improve selectivity and potency of a dendrimeric nanoparticle may be achieved by conjugation with an antibody that internalizes upon binding to target cells.

We synthesized and characterized a novel immunoconjugate, designated E1-G5/2, which was made by the DNL method to comprise half of a generation 5 (G5) PAMAM dendrimer (G5/2) site-specifically linked to a stabilized dimer of Fab derived from hRS7, a humanized antibody that is rapidly internalized upon binding to the Trop-2 antigen expressed on various solid cancers.

Methods

E1-G5/2 was prepared by combining two self-assembling modules, AD2-G5/2 and hRS7-Fab-DDD2, under mild redox conditions, followed by purification on a Protein L column. To make AD2-G5/2, we derivatized the AD2 peptide with a maleimide group to react with the single thiol generated from reducing a G5 PAMAM with a cystamine core and used reversed-phase HPLC to isolate AD2-G5/2. We produced hRS7-Fab-DDD2 as a fusion protein in myeloma cells, as described in the Examples above.

The molecular size, purity and composition of E1-G5/2 were analyzed by size-exclusion HPLC, SDS-PAGE, and Western blotting. The biological functions of E1-G5/2 were assessed by binding to an anti-idiotype antibody against hRS7, a gel retardation assay, and a DNase protection assay.

Results

E1-G5/2 was shown by size-exclusion HPLC to consist of a major peak (>90%) flanked by several minor peaks. The three constituents of E1-G5/2 (Fd-DDD2, the light chain, and AD2-G5/2) were detected by reducing SDS-PAGE and confirmed by Western blotting. Anti-idiotype binding analysis revealed E1-G5/2 contained a population of antibody-dendrimer conjugates of different size, all of which were capable of recognizing the anti-idiotype antibody, thus suggesting structural variability in the size of the purchased G5 dendrimer. Gel retardation assays showed E1-G5/2 was able to maximally condense plasmid DNA at a charge ratio of 6:1 (+/−), with the resulting dendriplexes completely protecting the complexed DNA from degradation by DNase I.

Conclusion

The DNL technique can be used to build dendrimer-based nanoparticles that are targetable with antibodies. Such agents have improved properties as carriers of drugs, plasmids or siRNAs for applications in vitro and in vivo. In preferred embodiments, anti-APC and/or anti-DC antibodies, such as anti-CD74 and/or anti-HLA-DR, may be utilized to deliver cytotoxic or cytostatic siRNA species to targeted DCs and/or APCs for therapy of GVHD and other immune dysfunctions.

### Example 10

### Maleimide AD2 Conjugate for DNL Dendrimers

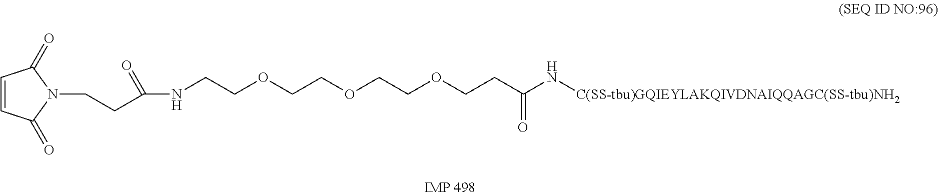

The peptide IMP 498 up to and including the PEG moiety was synthesized on a Protein Technologies PS3 peptide synthesizer by the Fmoc method on Sieber Amide resin (0.1 mmol scale). The maleimide was added manually by mixing the β-maleimidopropionic acid NHS ester with diisopropylethylamine and DMF with the resin for 4 hr. The peptide was cleaved from the resin with 15 mL TFA, 0.5 mL H₂O, 0.5 mL triisopropylsilane, and 0.5 mL thioanisole for 3 hr at room temperature. The peptide was purified by reverse phase HPLC using H₂O/CH₃CN TFA buffers to obtain about 90 mg of purified product after lyophilization.

### Synthesis of Reduced G5 Dendrimer (G5/2)

The G-5 dendrimer (10% in MeOH, Dendritic Nanotechnologies), 2.03 g, 7.03×10⁻⁶ mol was reduced with 0.1426 TCEP.HCl 1:1 MeOH/H₂O (˜4 mL) and stirred overnight at room temperature. The reaction mixture was purified by reverse phase HPLC on a C-18 column eluted with 0.1% TFA H₂O/CH₃CN buffers to obtain 0.0633 g of the desired product after lyophilization.

### Synthesis of G5/2 Dendrimer-AD2 Conjugate

The G5/2 Dendrimer, 0.0469 g (3.35×10⁻⁶ mol) was mixed with 0.0124 g of IMP 498 (4.4×10⁻⁶ mol) and dissolved in 1:1 MeOH/1M NaHCO₃ and mixed for 19 hr at room temperature followed by treatment with 0.0751 g dithiothreitol and 0.0441 g TCEP.HCl. The solution was mixed overnight at room temperature and purified on a C4 reverse phase HPLC column using 0.1% TFA H₂O/CH₃CN buffers to obtain 0.0033 g of material containing the conjugated AD2 and dendrimer as judged by gel electrophoresis and Western blot.

### Example 11

### Targeted Delivery of siRNA Using Protamine Linked Antibodies Summary

RNA interference (RNAi) has been shown to down-regulate the expression of various proteins such as HER2, VEGF, Raf-1, bcl-2, EGFR and numerous others in preclinical studies. Despite the potential of RNAi to silence specific genes, the full therapeutic potential of RNAi remains to be realized due to the lack of an effective delivery system to target cells in vivo.

To address this critical need, we developed novel DNL constructs having multiple copies of human protamine tethered to a tumor-targeting, internalizing hRS7 (anti-Trop-2) antibody for targeted delivery of siRNAs in vivo. A DDD2-L-thP1 module comprising truncated human protamine (thP1, residues 8 to 29 of human protamine 1) was produced, in which the sequences of DDD2 and thP1 were fused respectively to the N- and C-terminal ends of a humanized antibody light chain (not shown). The sequence of the truncated hP1 (thP1) is shown below. Reaction of DDD2-L-thP1 with the antibody hRS7-IgG-AD2 under mild redox conditions, as described in the Examples above, resulted in the formation of an E1-L-thP1 complex (not shown), comprising four copies of thP1 attached to the carboxyl termini of the hRS7 heavy chains.

tHP1
(SEQ ID NO: 97)
RSQSRSRYYRQRQRSRRRRRRS

The purity and molecular integrity of E1-L-thP1 following Protein A purification were determined by size-exclusion HPLC and SDS-PAGE (not shown). In addition, the ability of E1-L-thP1 to bind plasmid DNA or siRNA was demonstrated by the gel shift assay (not shown). E1-L-thP1 was effective at binding short double-stranded oligonucleotides (not shown) and in protecting bound DNA from digestion by nucleases added to the sample or present in serum (not shown).

The ability of the E1-L-thP1 construct to internalize siRNAs into Trop-2-expressing cancer cells was confirmed by fluorescence microscopy using FITC-conjugated siRNA and the human Calu-3 lung cancer cell line (not shown).

Methods

The DNL technique was employed to generate E1-L-thP1. The hRS7 IgG-AD module, constructed as described in the Examples above, was expressed in myeloma cells and purified from the culture supernatant using Protein A affinity chromatography. The DDD2-L-thP1 module was expressed as a fusion protein in myeloma cells and was purified by Protein L affinity chromatography. Since the CH3-AD2-IgG module possesses two AD2 peptides and each can bind to a DDD2 dimer, with each DDD2 monomer attached to a protamine moiety, the resulting E1-L-thP1 conjugate comprises four protamine groups. E1-L-thp1 was formed in nearly quantitative yield from the constituent modules and was purified to near homogeneity (not shown) with Protein A.

DDD2-L-thP1 was purified using Protein L affinity chromatography and assessed by size exclusion HPLC analysis and SDS-PAGE under reducing and nonreducing conditions (data not shown). A major peak was observed at 9.6 min (not shown). SDS-PAGE showed a major band between 30 and 40 kDa in reducing gel and a major band about 60 kDa (indicating a dimeric form of DDD2-L-thP1) in nonreducing gel (not shown). The results of Western blotting confirmed the presence of monomeric DDD2-L-tP1 and dimeric DDD2-L-tP1 on probing with anti-DDD antibodies (not shown).

To prepare the E1-L-thP1, hRS7-IgG-AD2 and DDD2-L-thP1 were combined in approximately equal amounts and reduced glutathione (final concentration 1 mM) was added. Following an overnight incubation at room temperature, oxidized glutathione was added (final concentration 2 mM) and the incubation continued for another 24 h. El-L-thP1 was purified from the reaction mixture by Protein A column chromatography and eluted with 0.1 M sodium citrate buffer (pH 3.5). The product peak was neutralized, concentrated, dialyzed with PBS, filtered, and stored in PBS containing 5% glycerol at 2 to 8° C. The composition of E1-L-thP1 was confirmed by reducing SDS-PAGE (not shown), which showed the presence of all three constituents (AD2-appended heavy chain, DDD2-L-htP1, and light chain).

The ability of DDD2-L-thP1 (not shown) and E1-L-thP1 (not shown) to bind DNA was evaluated by gel shift assay. DDD2-L-thP1 retarded the mobility of 500 ng of a linear form of 3-kb DNA fragment in 1% agarose at a molar ratio of 6 or higher (not shown). El-L-thP1 retarded the mobility of 250 ng of a linear 200-bp DNA duplex in 2% agarose at a molar ratio of 4 or higher (not shown), whereas no such effect was observed for hRS7-IgG-AD2 alone (not shown). The ability of E1-L-thP1 to protect bound DNA from degradation by exogenous DNase and serum nucleases was also demonstrated (not shown).

The ability of E1-L-thP1 to promote internalization of bound siRNA was examined in the Trop-2 expressing ME-180 cervical cell line (not shown). Internalization of the E1-L-thP1 complex was monitored using FITC conjugated goat anti-human antibodies. The cells alone showed no fluorescence (not shown). Addition of FITC-labeled siRNA alone resulted in minimal internalization of the siRNA (not shown). Internalization of E1-L-thP1 alone was observed in 60 minutes at 37° C. (not shown). E1-L-thP1 was able to effectively promote internalization of bound FITC-conjugated siRNA (not shown). E1-L-thP1 (10 μg) was mixed with FITC-siRNA (300 nM) and allowed to form E1-L-thPl-siRNA complexes which were then added to Trop-2-expressing Calu-3 cells. After incubation for 4 h at 37° C. the cells were checked for internalization of siRNA by fluorescence microscopy (not shown).

The ability of E1-L-thP1 to induce apoptosis by internalization of siRNA was examined. E1-L-thP1 (10 μg) was mixed with varying amounts of siRNA (AllStars Cell Death siRNA, Qiagen, Valencia, Calif.). The E1-L-thP1-siRNA complex was added to ME-180 cells. After 72 h of incubation, cells were trypsinized and annexin V staining was performed to evaluate apoptosis. The Cell Death siRNA alone or E1-L-thP1 alone had no effect on apoptosis (not shown). Addition of increasing amounts of E1-L-thP1-siRNA produced a dose-dependent increase in apoptosis (not shown). These results show that E1-L-thP1 could effectively deliver siRNA molecules into the cells and induce apoptosis of target cells.

Conclusions

The DNL technology provides a modular approach to efficiently tether multiple protamine molecules to the anti-Trop-2 hRS7 antibody resulting in the novel molecule E1-L-thP1. SDS-PAGE demonstrated the homogeneity and purity of E1-L-thP1. DNase protection and gel shift assays showed the DNA binding activity of E1-L-thP1. E1-L-thP1 internalized in the cells like the parental hRS7 antibody and was able to effectively internalize siRNA molecules into Trop-2-expressing cells, such as ME-180 and Calu-3.

The skilled artisan will realize that the DNL technique is not limited to any specific antibody or siRNA species. Rather, the same methods and compositions demonstrated herein can be used to make targeted delivery complexes comprising any antibody, any siRNA carrier and any siRNA species. The use of a bivalent IgG in targeted delivery complexes would result in prolonged circulating half-life and higher binding avidity to target cells, resulting in increased uptake and improved efficacy.

### Example 12

### Hexavalent DNL Constructs

The DNL technology described above for formation of trivalent DNL complexes was applied to generate hexavalent IgG-based DNL structures (HIDS). Because of the increased number of binding sites for target antigens, hexavalent constructs might be expected to show greater affinity and/or efficacy against target cells. Two types of modules, which were produced as recombinant fusion proteins, were combined to generate a variety of HIDS. Fab-DDD2 modules were as described for use in generating trivalent Fab structures (Rossi et al. Proc Natl Acad Sci USA.2006; 103(18): 6841-6). The Fab-DDD2 modules form stable homodimers that bind to AD2-containing modules. To generate HIDS, two types of IgG-AD2 modules were created to pair with the Fab-DDD2 modules: C-H-AD2-IgG and N-L-AD2-IgG.

C-H-AD2-IgG modules have an AD2 peptide fused to the carboxyl terminus (C) of the heavy (H) chain of IgG via a 9 amino acid residue peptide linker. The DNA coding sequences for the linker peptide followed by the AD2 peptide are coupled to the 3′ end of the CH3 (heavy chain constant domain 3) coding sequence by standard recombinant DNA methodologies, resulting in a contiguous open reading frame. When the heavy chain-AD2 polypeptide is co-expressed with a light chain polypeptide, an IgG molecule is formed possessing two AD2 peptides, which can therefore bind two Fab-DDD2 dimers. The C-H-AD2-IgG module can be combined with any Fab-DDD2 module to generate a wide variety of hexavalent structures composed of an Fc fragment and six Fab fragments. If the C-H-AD2-IgG module and the Fab-DDD2 module are derived from the same parental monoclonal antibody (MAb) the resulting HIDS is monospecific with 6 binding arms to the same antigen. If the modules are instead derived from two different MAbs then the resulting HIDS are bispecific, with two binding arms for the specificity of the C-H-AD2-IgG module and 4 binding arms for the specificity of the Fab-DDD2 module.

N-L-AD2-IgG is an alternative type of IgG-AD2 module in which an AD2 peptide is fused to the amino terminus (N) of the light (L) chain of IgG via a peptide linker. The L chain can be either Kappa (K) or Lambda (λ) and will also be represented as K. The DNA coding sequences for the AD2 peptide followed by the linker peptide are coupled to the 5′ end of the coding sequence for the variable domain of the L chain (V_{L}), resulting in a contiguous open reading frame. When the AD2-kappa chain polypeptide is co-expressed with a heavy chain polypeptide, an IgG molecule is formed possessing two AD2 peptides, which can therefore bind two Fab-DDD2 dimers. The N-L-AD2-IgG module can be combined with any Fab-DDD2 module to generate a wide variety of hexavalent structures composed of an Fc fragment and six Fab fragments.

The same technique has been utilized to produce DNL complexes comprising an IgG moiety attached to four effector moieties, such as cytokines. In an exemplary embodiment, an IgG moiety was attached to four copies of interferon-α2b. The antibody-cytokine DNL construct exhibited superior pharmacokinetic properties and/or efficacy compared to PEGylated forms of interferon-α2b.

### Example 13

### Generation of Hexavalent DNL Constructs

Generation of Hex-hA20

The DNL method was used to create Hex-hA20, a monospecific anti-CD20 HIDS, by combining C-H-AD2-hA20 IgG with hA20-Fab-DDD2. The Hex-hA20 structure contains six anti-CD20 Fab fragments and an Fc fragment, arranged as four Fab fragments and one IgG antibody. Hex-hA20 was made in four steps.

Step 1, Combination: A 210% molar equivalent of (hA20-Fab-DDD2)₂ was mixed with C-H-AD2-hA20 IgG. This molar ratio was used because two Fab-DDD2 dimers are coupled to each C-H-AD2-hA20 IgG molecule and an additional 10% excess of the former ensures that the coupling reaction is complete. The molecular weights of C-H-AD2-hA20 IgG and (hA20-Fab-DDD2)₂ are 168 kDa and 107 kDa, respectively. As an example, 134 mg of hA20-Fab-DDD2 would be mixed with 100 mg of C-H-AD2-hA20 IgG to achieve a 210% molar equivalent of the former. The mixture is typically made in phosphate buffered saline, pH 7.4 (PBS) with 1 mM EDTA.

Step 2, Mild Reduction: Reduced glutathione (GSH) was added to a final concentration of 1 mM and the solution is held at room temperature (16-25° C.) for 1-24 hours.

Step 3, Mild Oxidation: Following reduction, oxidized glutathione (GSSH) was added directly to the reaction mixture to a final concentration of 2 mM and the solution was held at room temperature for 1-24 hours.

Step 4, Isolation of the DNL product: Following oxidation, the reaction mixture was loaded directly onto a Protein-A affinity chromatography column. The column was washed with PBS and the Hex-hA20 was eluted with 0.1 M glycine, pH 2.5. Since excess hA20-Fab-DDD2 was used in the reaction, there was no unconjugated C-H-AD2-hA20 IgG, or incomplete DNL structures containing only one (hA20-Fab-DDD2)₂ moiety. The unconjugated excess hA20-Fab-DDD2 does not bind to the affinity resin. Therefore, the Protein A-purified material contains only the desired product.

The calculated molecular weight from the deduced amino acid sequences of the constituent polypeptides is 386 kDa. Size exclusion HPLC analysis showed a single protein peak with a retention time consistent with a protein structure of 375-400 kDa (not shown). SDS-PAGE analysis under non-reducing conditions showed a cluster of high molecular weight bands indicating a large covalent structure (not shown). SDS-PAGE under reducing conditions showed the presence of only the three expected polypeptide chains: the AD2-fused heavy chain (HC-AD2), the DDD2-fused Fd chain (Fd-DDD2), and the kappa chains (not shown).

Generation of Hex-hLL2

The DNL method was used to create a monospecific anti-CD22 HIDS (Hex-hLL2) by combining C-H-AD2-hLL2 IgG with hLL2-Fab-DDD2. The DNL reaction was accomplished as described above for Hex-hA20. The calculated molecular weight from the deduced amino acid sequences of the constituent polypeptides is 386 kDa. Size exclusion HPLC analysis showed a single protein peak with a retention time consistent with a protein structure of 375-400 kDa (not shown). SDS-PAGE analysis under non-reducing conditions showed a cluster of high molecular weight bands, which were eliminated under reducing conditions to leave only the three expected polypeptide chains: HC-AD2, Fd-DDD2, and the kappa chain (not shown).

Generation of DNL1 and DNL

The DNL method was used to create bispecific HIDS by combining C-H-AD2-hLL2 IgG with either hA20-Fab-DDD2 to obtain DNL1 or hMN-14-DDD2 to obtain DNL1C. DNL1 has four binding arms for CD20 and two for CD22. As hMN-14 is a humanized MAb to carcinoembryonic antigen (CEACAM5), DNL1C has four binding arms for CEACAM5 and two for CD22. The DNL reactions were accomplished as described for Hex-hA20 above.

For both DNL1 and DNL1C, the calculated molecular weights from the deduced amino acid sequences of the constituent polypeptides are ˜386 kDa. Size exclusion HPLC analysis showed a single protein peak with a retention time consistent with a protein structure of 375-400 kDa for each structure (not shown). SDS-PAGE analysis under non-reducing conditions showed a cluster of high molecular weight bands, which were eliminated under reducing conditions to leave only the three expected polypeptides: HC-AD2, Fd-DDD2, and the kappa chain (not shown).

Generation of DNL2 and DNL2C

The DNL method was used to create bispecific HIDS by combining C-H-AD2-hA20 IgG with either hLL2-Fab-DDD2 to obtain DNL2 or hMN-14-DDD2 to obtain DNL2C. DNL2 has four binding arms for CD22 and two for CD20. DNL2C has four binding arms for CEACAM5 and two for CD20. The DNL reactions were accomplished as described for Hex-hA20.

For both DNL2 and DNL2C, the calculated molecular weights from the deduced amino acid sequences of the constituent polypeptides are ˜386 kDa. Size exclusion HPLC analysis showed a single protein peak with a retention time consistent with a protein structure of 375-400 kDa for each structure (not shown). SDS-PAGE analysis under non-reducing conditions showed high molecular weight bands, but under reducing conditions consisted solely of the three expected polypeptides: HC-AD2, Fd-DDD2, and the kappa chain (not shown).

Generation of K-Hex-hA20

The DNL method was used to create a monospecific anti-CD20 HIDS (K-Hex-hA20) by combining N-L-AD2-hA20 IgG with hA20-Fab-DDD2. The DNL reaction was accomplished as described above for Hex-hA20.

The calculated molecular weight from the deduced amino acid sequences of the constituent polypeptides is 386 kDa. SDS-PAGE analysis under non-reducing conditions showed a cluster of high molecular weight bands, which under reducing conditions were composed solely of the four expected polypeptides: Fd-DDD2, H-chain, kappa chain, and AD2-kappa (not shown).

Generation of DNL3

A bispecific HIDS was generated by combining N-L-AD2-hA20 IgG with hLL2-Fab-DDD2. The DNL reaction was accomplished as described above for Hex-hA20. The calculated molecular weight from the deduced amino acid sequences of the constituent polypeptides is 386 kDa. Size exclusion HPLC analysis showed a single protein peak with a retention time consistent with a protein structure of 375-400 kDa (not shown). SDS-PAGE analysis under non-reducing conditions showed a cluster of high molecular weight bands that under reducing conditions showed only the four expected polypeptides: Fd-DDD2, H-chain, kappa chain, and AD2-kappa (not shown).

Stability in Serum

The stability of DNL1 and DNL2 in human serum was determined using a bispecific ELISA assay. The protein structures were incubated at 10 μg/ml in fresh pooled human sera at 37° C. and 5% CO₂ for five days. For day 0 samples, aliquots were frozen in liquid nitrogen immediately after dilution in serum. ELISA plates were coated with an anti-Id to hA20 IgG and bispecific binding was detected with an anti-Id to hLL2 IgG. Both DNL1 and DNL2 were highly stable in serum and maintained complete bispecific binding activity.

Binding Activity

The HIDS generated as described above retained the binding properties of their parental Fab/IgGs. Competitive ELISAs were used to investigate the binding avidities of the various HIDS using either a rat anti-idiotype MAb to hA20 (WR2) to assess the binding activity of the hA20 components or a rat anti-idiotype MAb to hLL2 (WN) to assess the binding activity of the hLL2 components. To assess hA20 binding, ELISA plates were coated with hA20 IgG and the HIDS were allowed to compete with the immobilized IgG for WR2 binding. To assess hLL2 binding, plates were coated with hLL2 IgG and the HIDS were allowed to compete with the immobilized IgG for WN binding. The relative amount of anti-Id bound to the immobilized IgG was detected using peroxidase-conjugated anti-Rat IgG.

Examining the relative CD20 binding avidities, DNL2, which has two CD20 binding groups, showed a similar binding avidity to hA20 IgG, which also has two CD20-binding arms (not shown). DNL1, which has four CD20-binding groups, had a stronger (˜4-fold) relative avidity than DNL2 or hA20 IgG (not shown). Hex-hA20, which has six CD20-binding groups, had an even stronger (˜10-fold) relative avidity than hA20 IgG (not shown).

Similar results were observed for CD22 binding. DNL1, which has two CD20 binding groups, showed a similar binding avidity to hLL2 IgG, which also has two CD22-binding arms (not shown). DNL2, which has four CD22-binding groups, had a stronger (>5-fold) relative avidity than DNL1 or hLL2 IgG. Hex-hLL2, which has six CD22-binding groups, had an even stronger (>10-fold) relative avidity than hLL2 IgG (not shown).

As both DNL2 and DNL3 contain two hA20 Fabs and four hLL2 Fabs, they showed similar strength in binding to the same anti-id antibody (not shown).

Some of the BIDS were observed to have potent anti-proliferative activity on lymphoma cell lines. DNL1, DNL2 and Hex-hA20 inhibited cell growth of Daudi Burkitt Lymphoma cells in vitro (not shown). Treatment of the cells with 10 nM concentrations was substantially more effective for the HIDS compared to rituximab (not shown). Using a cell counting assay, the potency of DNL1 and DNL2 was estimated to be more than 100-fold greater than that of rituximab, while the Hex-hA20 was shown to be even more potent (not shown). This was confirmed with an MTS proliferation assay in which dose-response curves were generated for Daudi cells treated with a range of concentrations of the HIDS (not shown). Compared to rituximab, the bispecific HIDS (DNL1 and DNL2) and Hex-hA20 were >100-fold and >10000-fold more potent, respectively.

### Example 14

### Ribonuclease Based DNL Immunotoxins Comprising Quadruple Ranpirnase (Rap) Conjugated to B-Cell Targeting Antibodies

We applied the DNL method to generate a novel class of immunotoxins, each of which comprises four copies of Rap site-specifically linked to a bivalent IgG. We combined a recombinant Rap-DDD module, produced in E. coli, with recombinant, humanized IgG-AD modules, which were produced in myeloma cells and targeted B-cell lymphomas and leukemias via binding to CD20 (hA20, veltuzumab), CD22 (hLL2, epratuzumab) or HLA-DR (hL243, IMMU-114), to generate 20-Rap, 22-Rap and C2-Rap, respectively. For each construct, a dimer of Rap was covalently tethered to the C-terminus of each heavy chain of the respective IgG. A control construct, 14-Rap, was made similarly, using labetuzumab (hMN-14), that binds to an antigen (CEACAM5) not expressed on B-cell lymphomas/leukemias.

Rap-DDD2
(SEQ ID NO: 99)
pQDWLTFQKKHITNTRDVDCDNIMSTNLFHCKDKNTFIYSRPEPVKAICKGIIASKNVLT
TSEFYLSDCNVTSRPCKYKLKKSTNKFCVTCENQAPVHFVGVGSCGGGGSLECGHIQIP
PGLTELLQGYTVEVLRQQPPDLVEFAVEYFTRLREARAVEHHHHHH

The deduced amino acid sequence of secreted Rap-DDD2 is shown above (SEQ ID NO:99). Rap, underlined; linker, italics; DDD2, bold; pQ, amino-terminal glutamine converted to pyroglutamate. Rap-DDD2 was produced in E. coli as inclusion bodies, which were purified by IMAC under denaturing conditions, refolded and then dialyzed into PBS before purification by Q-Sepharose anion exchange chromatography. SDS-PAGE under reducing conditions resolved a protein band with a Mr appropriate for Rap-DDD2 (18.6 kDa) (not shown). The final yield of purified Rap-DDD2 was 10 mg/L of culture.

The DNL method was employed to rapidly generate a panel of IgG-Rap conjugates. The IgG-AD modules were expressed in myeloma cells and purified from the culture supernatant using Protein A affinity chromatography. The Rap-DDD2 module was produced and mixed with IgG-AD2 to form a DNL complex. Since the CH3-AD2-IgG modules possess two AD2 peptides and each can tether a Rap dimer, the resulting IgG-Rap DNL construct comprises four Rap groups and one IgG. IgG-Rap is formed nearly quantitatively from the constituent modules and purified to near homogeneity with Protein A.

Prior to the DNL reaction, the CH3-AD2-IgG exists as both a monomer, and a disulfide-linked dimer (not shown). Under non-reducing conditions, the IgG-Rap resolves as a cluster of high molecular weight bands of the expected size between those for monomeric and dimeric CH3-AD2-IgG (not shown). Reducing conditions, which reduces the conjugates to their constituent polypeptides, shows the purity of the IgG-Rap and the consistency of the DNL method, as only bands representing heavy-chain-AD2 (HC-AD2), kappa light chain and Rap-DDD2 were visualized (not shown).

Reversed phase HPLC analysis of 22-Rap (not shown) resolved a single protein peak at 9.10 min eluting between the two peaks of CH3-AD2-IgG-hLL2, representing the monomeric (7.55 min) and the dimeric (8.00 min) forms. The Rap-DDD2 module was isolated as a mixture of dimer and tetramer (reduced to dimer during DNL), which were eluted at 9.30 and 9.55 min, respectively (not shown).

LC/MS analysis of 22-Rap was accomplished by coupling reversed phase HPLC using a C8 column with ESI-TOF mass spectrometry (not shown). The spectrum of unmodified 22-Rap identifies two major species, having either two G0F (G0F/G0F) or one GOF plus one G1F (G0F/G1F) N-linked glycans, in addition to some minor glycoforms (not shown). Enzymatic deglycosylation resulted in a single deconvoluted mass consistent with the calculated mass of 22-Rap (not shown). The resulting spectrum following reduction with TCEP identified the heavy chain-AD2 polypeptide modified with an N-linked glycan of the G0F or G1F structure as well as additional minor forms (not shown). Each of the three subunit polypeptides comprising 22-Rap were identified in the deconvoluted spectrum of the reduced and deglycosylated sample (not shown). The results confirm that both the Rap-DDD2 and HC-AD2 polypeptides have an amino terminal glutamine that is converted to pyroglutamate (pQ); therefore, 22-Rap has 6 of its 8 constituent polypeptides modified by pQ.

In vitro cytotoxicity was evaluated in three NHL cell lines. Each cell line expresses CD20 at a considerably higher surface density compared to CD22; however, the internalization rate for hLL2 (anti-CD22) is much faster than hA20 (anti-CD20). 14-Rap shares the same structure as 22-Rap and 20-Rap, but its antigen (CEACAM5) is not expressed by the NHL cells. Cells were treated continuously with IgG-Rap as single agents or with combinations of the parental MAbs plus rRap. Both 20-Rap and 22-Rap killed each cell line at concentrations above 1 nM, indicating that their action is cytotoxic as opposed to merely cytostatic (not shown). 20-Rap was the most potent IgG-Rap, suggesting that antigen density may be more important than internalization rate. Similar results were obtained for Daudi and Ramos, where 20-Rap (EC50˜0.1 nM) was 3-6-fold more potent than 22-Rap (not shown). The rituximab-resistant mantle cell lymphoma line, Jeko-1, exhibits increased CD20 but decreased CD22, compared to Daudi and Ramos. Importantly, 20-Rap exhibited very potent cytotoxicity (EC₅₀˜20 pM) in Jeko-1, which was 25-fold more potent than 22-Rap (not shown).

The DNL method provides a modular approach to efficiently tether multiple cytotoxins onto a targeting antibody, resulting in novel immunotoxins that are expected to show higher in vivo potency due to improved pharmacokinetics and targeting specificity. LC/MS, RP-HPLC and SDS-PAGE demonstrated the homogeneity and purity of IgG-Rap. Targeting Rap with a MAb to a cell surface antigen enhanced its tumor-specific cytotoxicity. Antigen density and internalization rate are both critical factors for the observed in vitro potency of IgG-Rap. In vitro results show that CD20-, CD22-, or HLA-DR-targeted IgG-Rap have potent biologic activity for therapy of B-cell lymphomas and leukemias.

### Example 15

### Production and Use of a DNL Construct Comprising Two Different Antibody Moieties and a Cytokine

In certain embodiments, the trimeric DNL constructs may comprise three different effector moieties, for example two different antibody moieties and a cytokine moiety. We report here the generation and characterization of the first bispecific MAb-IFNα, designated 20-C2-2b, which comprises two copies of IFN-α2b and a stabilized F(ab)₂ of hL243 (humanized anti-HLA-DR; IMMU-114) site-specifically linked to veltuzumab (humanized anti-CD20). In vitro, 20-C2-2b inhibited each of four lymphoma and eight myeloma cell lines, and was more effective than monospecific CD20-targeted MAb-IFNα or a mixture comprising the parental antibodies and IFNα in all but one (HLA-DR⁻/CD20⁻) myeloma line, suggesting that 20-C2-2b should be useful in the treatment of various hematopoietic disorders. The 20-C2-2b displayed greater cytotoxicity against KMS12-BM (CD20^{+/HLA-DR}⁺ myeloma) than monospecific MAb-IFNα that targets only HLA-DR or CD20, indicating that all three components in 20-C2-2b can contribute to toxicity. Our findings indicate that a given cell's responsiveness to MAb-IFNα depends on its sensitivity to IFNα and the specific antibodies, as well as the expression and density of the targeted antigens.

Because 20-C2-2b has antibody-dependent cellular cytotoxicity (ADCC), but not CDC, and can target both CD20 and HLA-DR, it is useful for therapy of a broad range of hematopoietic disorders that express either or both antigens. The skilled artisan will realize that similar constructs targeting CD74 and HLA-DR may be constructed by DNL and used for therapy of GVHD.

Antibodies

The abbreviations used in the following discussion are: 20 (C_{H}3-AD2-IgG-v-mab, anti-CD20 IgG DNL module); C2 (C_{H}1-DDD2-Fab-hL243, anti-HLA-DR Fab₂ DNL module); 2b (dimeric IFNα2B-DDD2 DNL module); 734 (anti-in-DTPA IgG DNL module used as non-targeting control). The following MAbs were provided by Immunomedics, Inc.: veltuzumab or v-mab (anti-CD20 IgG₁), hL243γ4p (Immu-114, anti-HLA-DR IgG₄), a murine anti-IFNα MAb, and rat anti-idiotype MAbs to v-mab (WR2) and hL243 (WT).

DNL Constructs

Monospecific MAb-IFNα (20-2b-2b, 734-2b-2b and C2-2b-2b) and the bispecific HexAb (20-C2-C2) were generated by combination of an IgG-AD2-module with DDD2-modules using the DNL method, as described in the preceding Examples. The 734-2b-2b, which comprises tetrameric IFNα2b and MAb h734 [anti-Indium-DTPA IgG₁], was used as a non-targeting control MAb-1FNα.

The construction of the mammalian expression vector as well as the subsequent generation of the production clones and the purification of C_{H}3-AD2-IgG-v-mab are disclosed in the preceding Examples. The expressed recombinant fusion protein has the AD2 peptide linked to the carboxyl terminus of the C_{H}3 domain of v-mab via a 15 amino acid long flexible linker peptide. Co-expression of the heavy chain-AD2 and light chain polypeptides results in the formation of an IgG structure equipped with two AD2 peptides. The expression vector was transfected into Sp/ESF cells (an engineered cell line of Sp2/0) by electroporation. The pdHL2 vector contains the gene for dihydrofolate reductase, thus allowing clonal selection, as well as gene amplification with methotrexate (MTX). Stable clones were isolated from 96-well plates selected with media containing 0.2 μM MTX. Clones were screened for C_{H}3-AD2-IgG-vmab productivity via a sandwich ELISA. The module was produced in roller bottle culture with serum-free media.

The DDD-module, IFNα2b-DDD2, was generated as discussed above by recombinant fusion of the DDD2 peptide to the carboxyl terminus of human IFNα2b via an 18 amino acid long flexible linker peptide. As is the case for all DDD-modules, the expressed fusion protein spontaneously forms a stable homodimer

The C_{H}1-DDD2-Fab-hL243 expression vector was generated from hL243-IgG-pdHL2 vector by excising the sequence for the C_{H}1-Hinge-C_{H}2-C_{H}3 domains with SacII and EagI restriction enzymes and replacing it with a 507 by sequence encoding C_{H}1-DDD2, which was excised from the C-DDD2-hMN-14-pdHL2 expression vector with the same enzymes. Following transfection of C_{H}1-DDD2-Fab-hL243-pdHL2 into Sp/ESF cells by electroporation, stable, MTX-resistant clones were screened for productivity via a sandwich ELISA using 96-well microtiter plates coated with mouse anti-human kappa chain to capture the fusion protein, which was detected with horseradish peroxidase-conjugated goat anti-human Fab. The module was produced in roller bottle culture.

Roller bottle cultures in serum-free H-SFM media and fed-batch bioreactor production resulted in yields comparable to other IgG-AD2 modules and cytokine-DDD2 modules generated to date. C_{H}3-AD2-IgG-v-mab and IFNα2b-DDD2 were purified from the culture broths by affinity chromatography using MABSELECT™ (GE Healthcare) and HIS-SELECT® HF Nickel Affinity Gel (Sigma), respectively, as described previously (Rossi et al., Blood 2009, 114:3864-71). The culture broth containing the C_{H}1-DDD2-Fab-hL243 module was applied directly to KAPPASELECT® affinity gel (GE-Healthcare), which was washed to baseline with PBS and eluted with 0.1 M Glycine, pH 2.5.

The purity of the DNL modules was assessed by SDS-PAGE and SE-HPLC (not shown). Analysis under non-reducing conditions showed that, prior to the DNL reaction, IFNα2b-DDD2 and C_{H}1-DDD2-Fab-hL243 exist as disulfide-linked dimers (not shown). This phenomenon, which is always seen with DDD-modules, is beneficial, as it protects the reactive sulfhydryl groups from irreversible oxidation. In comparison, C_{H}3-AD2-IgG-v-mab (not shown) exists as both a monomer and a disulfide-linked dimer, and is reduced to monomer during the DNL reaction. SE-HPLC analyses agreed with the non-reducing SDS-PAGE results, indicating monomeric species as well as dimeric modules that were converted to monomeric forms upon reduction (not shown). The sulfhydryl groups are protected in both forms by participation in disulfide bonds between AD2 cysteine residues. Reducing SDS-PAGE demonstrated that each module was purified to near homogeneity and identified the component polypeptides comprising each module (not shown). For C_{H}3-AD2-IgG-v-mab, heavy chain-AD2 and kappa light chains were identified. hL243-Fd-DDD2 and kappa light chain polypeptides were resolved for C_{H}1-DDD2-Fab-hL243 (not shown). One major and one minor band were resolved for IFNα2b-DDD2 (not shown), which were determined to be non-glycosylated and O-glycosylated species, respectively.

Generation of 20-C2-2b by DNL

Three DNL modules (C_{H}3-AD2-IgG-v-mab, C_{H}1-DDD2-Fab-hL243, and IFN-α2b-DDD2) were combined in equimolar quantities to generate the bsMAb-IFNα, 20-C2-2b. Following an overnight docking step under mild reducing conditions (1 mM reduced glutathione) at room temperature, oxidized glutathione was added (2 mM) to facilitate disulfide bond formation (locking). The 20-C2-2b was purified to near homogeneity using three sequential affinity chromatography steps. Initially, the DNL mixture was purified with Protein A (MABSELECT™), which binds the C_{H}3-AD2-IgG-v-MAb group and eliminates un-reacted IFNα2b-DDD2 or C_{H}1-DDD2-Fab-hL243. The Protein A-bound material was further purified by IMAC using HIS-SELECT® HF Nickel Affinity Gel, which binds specifically to the IFNα2b-DDD2 moiety and eliminates any constructs lacking this group. The final process step, using an hL243-anti-idiotype affinity gel removed any molecules lacking C_{H}1-DDD2-Fab-hL243.

The skilled artisan will realize that affinity chromatography may be used to purify DNL complexes comprising any combination of effector moieties, so long as ligands for each of the three effector moieties can be obtained and attached to the column material. The selected DNL construct is the one that binds to each of three columns containing the ligand for each of the three effector moieties and can be eluted after washing to remove unbound complexes.

The following Example is representative of several similar preparations of 20-C2-2b. Equimolar amounts of C_{H}3-AD2-IgG-v-mab (15 mg), C_{H}1-DDD2-Fab-hL243 (12 mg), and IFN-α2b-DDD2 (5 mg) were combined in 30-mL reaction volume and 1 mM reduced glutathione was added to the solution. Following 16 h at room temperature, 2 mM oxidized glutathione was added to the mixture, which was held at room temperature for an additional 6 h. The reaction mixture was applied to a 5-mL Protein A affinity column, which was washed to baseline with PBS and eluted with 0.1 M Glycine, pH 2.5. The eluate, which contained -20 mg protein, was neutralized with 3 M Tris-HCl, pH 8.6 and dialyzed into HIS-SELECT® binding buffer (10 mM imidazole, 300 mM NaCl, 50 mM NaH₂PO₄, pH 8.0) prior to application to a 5-mL HIS-SELECT® IMAC column. The column was washed to baseline with binding buffer and eluted with 250 mM imidazole, 150 mM NaCl, 50 mM NaH₂PO₄, pH 8.0.

The MAC eluate, which contained ˜11.5 mg of protein, was applied directly to a WP (anti-hL243) affinity column, which was washed to baseline with PBS and eluted with 0.1 M glycine, pH 2.5. The process resulted in 7 mg of highly purified 20-C2-2b. This was approximately 44% of the theoretical yield of 20-C2-2b, which is 50% of the total starting material (16 mg in this example) with 25% each of 20-2b-2b and 20-C2-C2 produced as side products.

Generation and Characterization of 20-C2-2b

The bispecific MAb-IFNα was generated by combining the IgG-AD2 module, C_{H}3-AD2-IgG-v-mab, with two different dimeric DDD-modules, C_{H}1-DDD2-Fab-hL243 and IFNα2b-DDD2. Due to the random association of either DDD-module with the two AD2 groups, two side-products, 20-C2-C2 and 20-2b-2b are expected to form, in addition to 20-C2-2b.

Non-reducing SDS-PAGE (not shown) resolved 20-C2-2b (˜305 kDa) as a cluster of bands positioned between those of 20-C2-C2 (˜365 kDa) and 20-2b-2b (255 kDa). Reducing SDS-PAGE resolved the five polypeptides (v-mab HC-AD2, hL243 Fd-DDD2, IFNα2b-DDD2 and co-migrating v-mab and hL243 kappa light chains) comprising 20-C2-2b (not shown). IFNα2b-DDD2 and hL243 Fd-DDD2 are absent in 20-C2-C2 and 20-2b-2b. MABSELECT™ binds to all three of the major species produced in the DNL reaction, but removes any excess IFNα2b-DDD2 and C_{H}1-DDD2-Fab-hL243. The HIS-SELECT® unbound fraction contained mostly 20-C2-C2 (not shown). The unbound fraction from WT affinity chromatography comprised 20-2b-2b (not shown). Each of the samples was subjected to SE-HPLC and immunoreactivity analyses, which corroborated the results and conclusions of the SDS-PAGE analysis.

Following reduction of 20-C2-2b, its five component polypeptides were resolved by RP-HPLC and individual ESI-TOF deconvoluted mass spectra were generated for each peak (not shown). Native, but not bacterially-expressed recombinant IFNα2, is O-glycosylated at Thr-106 (Adolf et al., Biochem J 1991;276 (Pt 2):511-8). We determined that ˜15% of the polypeptides comprising the IFNα2b-DDD2 module are O-glycosylated and can be resolved from the non-glycosylated polypeptides by RP-HPLC and SDS-PAGE (not shown). LC/MS analysis of 20-C2-2b identified both the O-glycosylated and non-glycosylated species of IFNα2b-DDD2 with mass accuracies of 15 ppm and 2 ppm, respectively (not shown). The observed mass of the O-glycosylated form indicates an O-linked glycan having the structure NeuGc-NeuGc-Gal-GalNAc, which was also predicted (<1 ppm) for 20-2b-2b (not shown). LC/MS identified both v-mab and hL243 kappa chains as well as hL243-Fd-DDD2 (not shown) as single, unmodified species, with observed masses matching the calculated ones (<35 ppm). Two major glycoforms of v-mab HC-AD2 were identified as having masses of 53,714.73 (70%) and 53,877.33 (30%), indicating G0F and G1F N-glycans, respectively, which are typically associated with IgG (not shown). The analysis also confirmed that the amino terminus of the HC-AD2 is modified to pyroglutamate, as predicted for polypeptides having an amino terminal glutamine.

SE-HPLC analysis of 20-C2-2b resolved a predominant protein peak with a retention time (6.7 min) consistent with its calculated mass and between those of the larger 20-C2-C2 (6.6 min) and smaller 20-2b-2b (6.85 min), as well as some higher molecular weight peaks that likely represent non-covalent dimers formed via self-association of IFNα2b (not shown).

Immunoreactivity assays demonstrated the homogeneity of 20-C2-2b with each molecule containing the three functional groups (not shown). Incubation of 20-C2-2b with an excess of antibodies to any of the three constituent modules resulted in quantitative formation of high molecular weight immune complexes and the disappearance of the 20-C2-2b peak. The HIS-SELECT® and WT affinity unbound fractions were not immunoreactive with WT and anti-IFNα, respectively (not shown). The MAb-IFNα showed similar binding avidity to their parental MAbs (not shown).

IFNα Biological Activity

The specific activities for various MAb-IFNα were measured using a cell-based reporter gene assay and compared to peginterferon alfa-2b (not shown). Expectedly, the specific activity of 20-C2-2b (2454 IU/pmol), which has two IFNα2b groups, was significantly lower than those of 20-2b-2b (4447 IU/pmol) or 734-2b-2b (3764 IU/pmol), yet greater than peginterferon alfa-2b (P<0.001). The difference between 20-2b-2b and 734-2b-2b was not significant. The specific activity among all agents varies minimally when normalized to IU/pmol of total IFNα. Based on these data, the specific activity of each IFNα2b group of the MAb-IFNα is approximately 30% of recombinant IFNα2b (˜4000 IU/pmol).

In the ex-vivo setting, the 20-C2-2b DNL construct depleted lymphoma cells more effectively than normal B cells and had no effect on T cells (not shown). However, it did efficiently eliminate monocytes (not shown). Where v-mab had no effect on monocytes, depletion was observed following treatment with hL243α4p and MAb-IFNα, with 20-2b-2b and 734-2b-2b exhibiting similar toxicity (not shown). Therefore, the predictably higher potency of 20-C2-2b is attributed to the combined actions of anti-HLA-DR and IFNα, which may be augmented by HLA-DR targeting. These data suggest that monocyte depletion may be a pharmacodynamic effect associated anti-HLA-DR as well as IFNα therapy; however, this side affect would likely be transient because the monocyte population should be repopulated from hematopoietic stem cells.

The skilled artisan will realize that the approach described here to produce and use bispecific immunocytokine, or other DNL constructs comprising three different effector moieties, may be utilized with any combinations of antibodies, antibody fragments, cytokines or other effectors that may be incorporated into a DNL construct.

It will be readily apparent to one skilled in the art that varying substitutions and modifications may be made to the invention disclosed herein without departing from the scope and spirit of the invention. Thus, such additional embodiments are within the scope of the present invention.

What is claimed is:

1. A method of killing antigen-presenting cells or dendritic cells comprising:
a. exposing the antigen-presenting cell or dendritic cell to an anti-HLA-DR and/or anti-CD74 antibody or antigen-binding fragment thereof; and
b. killing the antigen-presenting cell or dendritic cell.

2. The method of claim 1, wherein the anti-CD74 antibody or fragment thereof competes for binding to CD74 with, or binds to the same epitope of CD74 as, a murine LL1 antibody comprising the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6).

3. The method of claim 1, wherein the anti-CD74 antibody or fragment thereof comprises the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6).

4. The method of claim 1, wherein the anti-HLA-DR antibody or fragment thereof competes for binding to HLA-DR with, or binds to the same epitope of HLA-DR as, a murine L243 antibody comprising the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12).

5. The method of claim 1, wherein the anti-HLA-DR antibody or fragment thereof comprises the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12).

6. The method of claim 1, wherein the antigen-presenting cell or dendritic cell is exposed to a first antibody or fragment thereof that binds to CD74 or HLA-DR and to a second antibody or fragment thereof that binds to an antigen expressed by antigen-presenting cells, dendritic cells or B-cells.

7. The method of claim 6, wherein the antigen is selected from the group consisting of CD19, CD20, CD22, CD34, CD45, CD74, CD209, TLR 2 (toll-like receptor 2), TLR 4, TLR 7, TLR 9, BDCA-2, BDCA-3, BDCA-4, and HLA-DR.

8. The method of claim 6, wherein the first antibody or fragment thereof binds to CD74 and the second antibody or fragment thereof binds to HLA-DR.

9. The method of claim 1, further comprising killing myeloid dendritic cell type 1 (mDC1) and type 2 (mDC2) and not killing plasmacytoid dendritic cells (pDCs), monocytes or T cells.

10. The method of claim 1, further comprising killing all subsets of APCs, including mDCs, pDCs, B cells and monocytes, without killing T cells.

11. The method of claim 1, further comprising suppressing proliferation of allo-reactive T cells, while preserving cytomegalovirus (CMV)-specific, CD8⁺ memory T cells.

12. The method of claim 1, wherein the anti-CD74 antibody is milatuzumab.

13. The method of claim 1, wherein the anti-CD74 or anti-HLA-DR antibody or fragment thereof is a naked antibody or fragment thereof.

14. The method of claim 13, further comprising exposing the cell to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

15. The method of claim 1, wherein the anti-CD74 or anti-HLA-DR antibody or fragment thereof is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

16. The method of claim 15, wherein the anti-CD74 or anti-HLA-DR antibody or fragment thereof is conjugated to a second antibody or fragment thereof to form a bispecific antibody.

17. The method of claim 16, wherein the bispecific antibody is a dock-and-lock complex.

18. The method of claim 15, wherein the therapeutic agent is selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

19. The method of claim 14, wherein the therapeutic agent is bortezomib.

20. The method of claim 15, wherein the therapeutic agent is a radionuclide selected from the group consisting of ^{103m}Rh, ¹⁰³Ru, ¹⁰⁵Rh, ¹⁰⁵Ru, ¹⁰⁷hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁵At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

21. The method of claim 15, wherein the therapeutic agent is an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

22. The method of claim 15, wherein the therapeutic agent is an immunomodulator selected from the group consisting of erythropoietin, thrombopoietin tumor necrosis factor-α(TNF), TNF-β, granulocyte-colony stimulating factor (G-CSF), granulocyte macrophage-colony stimulating factor (GM-CSF), interferon-α, interferon-β, interferon-γ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin and LT.

23. A method of treating graft-versus-host disease (GVHD) comprising:
a. administering an anti-HLA-DR and/or anti-CD74 antibody or antigen-binding fragment thereof to a subject; and
b. depleting antigen-presenting cells and/or dendritic cells in the subject.

24. The method of claim 23, wherein the anti-CD74 antibody or fragment thereof competes for binding to CD74 with, or binds to the same epitope of CD74 as, a murine LL1 antibody comprising the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6).

25. The method of claim 23, wherein the anti-CD74 antibody or fragment thereof comprises the light chain CDR sequences CDR1 (RSSQSLVHRNGNTYLH; SEQ ID NO:1), CDR2 (TVSNRFS; SEQ ID NO:2), and CDR3 (SQSSHVPPT; SEQ ID NO:3) and the heavy chain variable region CDR sequences CDR1 (NYGVN; SEQ ID NO:4), CDR2 (WINPNTGEPTFDDDFKG; SEQ ID NO:5), and CDR3 (SRGKNEAWFAY; SEQ ID NO:6).

26. The method of claim 23, wherein the anti-HLA-DR antibody or fragment thereof competes for binding to HLA-DR with, or binds to the same epitope of HLA-DR as, a murine L243 antibody comprising the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12).

27. The method of claim 23, wherein the anti-HLA-DR antibody or fragment thereof comprises the heavy chain CDR sequences CDR1 (NYGMN, SEQ ID NO:7), CDR2 (WINTYTREPTYADDFKG, SEQ ID NO:8), and CDR3 (DITAVVPTGFDY, SEQ ID NO:9) and the light chain CDR sequences CDR1 (RASENIYSNLA, SEQ ID NO:10), CDR2 (AASNLAD, SEQ ID NO:11), and CDR3 (QHFWTTPWA, SEQ ID NO:12).

28. The method of claim 23, further comprising administering to the subject a first antibody or fragment thereof that binds to CD74 or HLA-DR and to a second antibody or fragment thereof that binds to an antigen expressed by antigen-presenting cells, dendritic cells or B-cells.

29. The method of claim 28, wherein the antigen is selected from the group consisting of CD19, CD20, CD22, CD34, CD45, CD74, CD209, TLR 2 (toll-like receptor 2), TLR 4, TLR 7, TLR 9, BDCA-2, BDCA-3, BDCA-4, and HLA-DR.

30. The method of claim 28, wherein the first antibody or fragment thereof binds to CD74 and the second antibody or fragment thereof binds to HLA-DR.

31. The method of claim 23, further comprising depleting myeloid dendritic cell type 1 (mDC1) and type 2 (mDC2) and not depleting plasmacytoid dendritic cells (pDCs), monocytes or T cells.

32. The method of claim 23, further comprising depleting all subsets of APCs, including mDCs, pDCs, B cells and monocytes, without depleting T cells.

33. The method of claim 23, further comprising suppressing proliferation of allo-reactive T cells, while preserving cytomegalovirus (CMV)-specific, CD8⁺ memory T cells.

34. The method of claim 23, wherein the anti-CD74 antibody is milatuzumab.

35. The method of claim 23, wherein the anti-CD74 or anti-HLA-DR antibody or fragment thereof is a naked antibody or fragment thereof.

36. The method of claim 35, further comprising exposing the cell to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

37. The method of claim 23, wherein the anti-CD74 or anti-HLA-DR antibody or fragment thereof is conjugated to at least one therapeutic agent selected from the group consisting of a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide, an antisense molecule, a siRNA, a second antibody and a second antibody fragment.

38. The method of claim 37, wherein the anti-CD74 or anti-HLA-DR antibody or fragment thereof is conjugated to a second antibody or fragment thereof to form a bispecific antibody.

39. The method of claim 38, wherein the bispecific antibody is a dock-and-lock complex.

40. The method of claim 37, wherein the therapeutic agent is selected from the group consisting of aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, and Pseudomonas endotoxin.

41. The method of claim 36, wherein the therapeutic agent is bortezomib.

42. The method of claim 37, wherein the therapeutic agent is a radionuclide selected from the group consisting of ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo and ^{99m}Tc.

43. The method of claim 37, wherein the therapeutic agent is an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

44. The method of claim 37, wherein the therapeutic agent is an immunomodulator selected from the group consisting of erythropoietin, thrombopoietin tumor necrosis factor-α(TNF), TNF-β, granulocyte-colony stimulating factor (G-CSF), granulocyte macrophage-colony stimulating factor (GM-CSF), interferon-α, interferon-β, interferon-γ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin and LT.

45. The method of claim 23, wherein the GVHD is acute GVHD or chronic GVHD.

46. The method of claim 1, wherein the antibody fragment is selected from the group consisting of F(ab′)₂, F(ab)₂, Fab′, Fab, Fv, scFv and single domain antibody.

47. The method of claim 1, wherein the anti-CD74 or anti-HLA-DR antibody is a chimeric, humanized or human antibody.

48. A dock-and-lock (DNL) complex of use to treat GVHD comprising:
a. a first fusion protein comprising an anti-HLA-DR or anti-CD74 antibody or antigen-binding fragment thereof; and
b. a second fusion protein comprising an effector moiety.

49. The complex of claim 48, wherein each fusion protein further comprises a peptide selected from the group consisting of (i) a dimerization and docking domain (DDD) of human protein kinase A (PICA) RIα, RIβ, RIIα or RIIβ; and (ii) an anchoring domain (AD) of an A-kinase anchoring protein (AKAP); and
wherein two copies of the DDD form a dimer that binds to one copy of the AD.

50. The complex of claim 48, further comprising at least one therapeutic agent.

51. The complex of claim 48, wherein the first fusion protein comprises an anti-HLA-DR antibody or antigen-binding fragment thereof and the second fusion protein comprises an anti-CD74 antibody or fragment thereof.

52. The complex of claim 48, wherein the effector moiety is selected from the group consisting of an antibody, an antigen-binding antibody fragment, a toxin, a cytokine and a siRNA carrier.

53. The complex of claim 52, wherein the effector moiety is a siRNA carrier and the complex further comprises at least one siRNA.
